(19) United States
(12) Patent Application Publication
(10) Pub. No.: US 2007/0099239 A1
(43) Pub. Date: May 3, 2007

(54) Methods and compositions for diagnosis and monitoring of atherosclerotic cardiovascular disease

(76) Inventors: Raymond Tabibiazar, Stanford, CA (US); Philip S. Tsao, Los Altos, CA (US); Thomas Quertermous, Stanford, CA (US); Brit Katzen Turnbull, Stanford, CA (US); Richard A. Olshen, Stanford, CA (US); Evangelos Hytopoulos, San Mateo, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/473,826

(22) Filed: Jun. 23, 2006

Related U.S. Application Data
(60) Provisional application No. 60/693,756, filed on Jun. 24, 2005.

(51) Int. Cl. G01N 33/53; G06F 19/00

(52) U.S. Cl. 435/7.1; 702/19

(57) ABSTRACT

The present invention identifies circulating proteins that are differentially expressed in atherosclerosis. Circulating levels of these proteins, particularly as a panel of proteins, can discriminate patients with acute myocardial infarction from those with stable exertional angina and from those with no history of atherosclerotic cardiovascular disease. Such levels can also predict cardiovascular events, determine the effectiveness of therapy, stage disease, and the like. For example, these markers are useful as surrogate biomarkers of clinical events needed for development of vascular specific pharmaceutical agents.

### CROSS REFERENCE TO RELATED APPLICATIONS

This application claims the benefit of U.S. Provisional Application No. 60/693,756, filed Jun. 24, 2005, the entire disclosure of which is hereby incorporated by reference in its entirety for all purposes.

### SEQUENCE LISTING

The present specification incorporates herein by reference, each in its entirety, the sequence information on the Compact Disks (CDs) labeled Copy 1 and Copy 2. The CDs are formatted on IBM-PC, with operating system compatibility with MS-Windows. The files on each of the CDs are as follows: Copy 1—Seqlist.txt 614 KB created Jun. 23, 2006; and Copy 2—Seqlist.txt 614 KB created Jun. 23, 2006.

### BACKGROUND OF THE INVENTION

1. Field of the Invention

This application is directed to the fields of bioinformatics and atherosclerotic disease. In particular this invention relates to methods and compositions for diagnosing, monitoring, and development of therapeutics for atherosclerotic disease.

2. Description of the Related Art

As our ability to provide early and accurate diagnosis followed by aggressive treatment has been limited, atherosclerotic cardiovascular disease (ASCVD) remains the primary cause of morbidity and mortality worldwide. Patients with ASCVD represent a heterogeneous group of individuals, with a disease that progresses at different rates and in distinctly different patterns. Despite appropriate evidence-based treatments for patients with ASCVD, recurrence and mortality rates remain 2-4% per year. Also, the full benefits of primary prevention are unrealized due to our inability to identify accurately those patients who would benefit from aggressive risk reduction.

Whereas certain disease markers have been shown to predict outcome or response to therapy at a population level, they are not sufficiently sensitive or specific to provide adequate clinical utility in an individual patient. As a result, the first clinical presentation for more than half of the patients with coronary artery disease is either myocardial infarction or death.

Physical examination and current diagnostic tools cannot accurately determine an individual's risk for suffering a complication of ASCVD. Known risk factors such as hypertension, hyperlipidemia, diabetes, family history, and smoking do not establish the diagnosis of atherosclerosis disease. Diagnostic modalities which rely on anatomical data (such as coronary angiography, coronary calcium score, CT or MRI angiography) lack information on the biological activity of the disease process and can be poor predictors of future cardiac events. Functional assessment of endothelial function can be non-specific and unrelated to the presence of atherosclerotic disease process, although some data has demonstrated the prognostic value of these measurements. Individual biomarkers, such as the lipid and inflammatory markers, have been shown to predict outcome and response to therapy in patients with ASCVD and some are utilized as important risk factors for developing atherosclerotic disease. Nonetheless, up to this point, no single biomarker is sufficiently specific to provide adequate clinical utility for the diagnosis of ASCVD in an individual patient.

### Complex Nature of Atherosclerotic Cardiovascular Disease

In general, atherosclerosis is believed to be a complex disease involving multiple biological pathways. Variations in the natural history of the atherosclerotic disease process, as well as differential response to risk factors and variations in the individual response to therapy, reflect in part differences in genetic background and their intricate interactions with the environmental factors that are responsible for the initiation and modification of the disease. Atherosclerotic disease is also influenced by the complex nature of the cardiovascular system itself where anatomy, function and biology all play important roles in health as well as disease. Given such complexities, it is unlikely that an individual marker or approach will yield sufficient information to capture the true nature of the disease process.

### Single Biomarker Approach: Inflammation

Inflammation has been implicated in all stages of ASCVD and is considered to be a major part of the pathophysiological basis of atherogenesis, providing a potential marker of the disease process. Elevated circulating inflammatory biomarkers have been shown to stratify cardiovascular risk and assess response to therapy in large epidemiological studies. Currently, while general markers of inflammation are potentially useful in risk stratification, they are not adequate to identify the presence of CAD in an individual, due a lack of specificity for many markers. For similar reasons, the general markers of inflammation such as C-reactive protein (CRP) and erythrocyte sedimentation rate (ESR) have long been abandoned as specific diagnostic markers in other inflammatory diseases such as lupus and rheumatoid arthritis, although they remain important markers for risk stratification and response to therapy in clinical practice.

It is also possible that the heterogeneity of the individual response to environmental risk factors induces a high variability in ASCVD marker concentration. In this context, biological information carried by a single inflammatory protein cannot be sufficient in providing a comprehensive representation of the vascular inflammatory state, and may not be able to accurately identify the presence or extent of the disease.

### Pathophysiological Basis of Atherosclerosis

Atherosclerotic plaque consists of accumulated intracellular and extracellular lipids, smooth muscle cells, connective tissue, and glycosaminoglycans. The earliest detectable lesion of atherosclerosis is the fatty streak, consisting of lipid-laden foam cells, which are macrophages that have migrated as monocytes from the circulation into the subendothelial layer of the intima, which later evolves into the fibrous plaque, consisting of intimal smooth muscle cells surrounded by connective tissue and intracellular and extracellular lipids.

Interrelated hypotheses have been proposed to explain the pathogenesis of atherosclerosis. The lipid hypothesis postulates that an elevation in plasma LDL levels results in penetration of LDL into the arterial wall, leading to lipid accumulation in smooth muscle cells and in macrophages. LDL also augments smooth muscle cell hyperplasia and migration into the subintimal and intimal region in response to growth factors. LDL is modified or oxidized in this environment and is rendered more atherogenic. The modified or oxidized LDL is chemotactic to monocytes, promoting their migration into the intima, their early appearance in the fatty streak, and their transformation and retention in the subintimal compartment as macrophages. Scavenger receptors on the surface of macrophages facilitate the entry of oxidized LDL into these cells, transferring them into lipid-laden macrophages and foam cells. Oxidized LDL is also cytotoxic to endothelial cells and may be responsible for their dysfunction or loss from the more advanced lesion.

The chronic endothelial injury hypothesis postulates that endothelial injury by various mechanisms produces loss of endothelium, adhesion of platelets to subendothelium, aggregation of platelets, chemotaxis of monocytes and T-cell lymphocytes, and release of platelet-derived and monocyte-derived growth factors that induce migration of smooth muscle cells from the media into the intima, where they replicate, synthesize connective tissue and proteoglycans, and form a fibrous plaque. Other cells, e.g. macrophages, endothelial cells, arterial smooth muscle cells, also produce growth factors that can contribute to smooth muscle hyperplasia and extracellular matrix production.

Endothelial dysfunction includes increased endothelial permeability to lipoproteins and other plasma constituents, expression of adhesion molecules and elaboration of growth factors that lead to increased adherence of monocytes, macrophages and T lymphocytes. These cells may migrate through the endothelium and situate themselves within the subendothelial layer. Foam cells also release growth factors and cytokines that promote migration of smooth muscle cells and stimulate neointimal proliferation, continue to accumulate lipid and support endothelial cell dysfunction. Clinical and laboratory studies have shown that inflammation plays a major role in the initiation, progression and destabilization of atheromas.

The “autoimmune” hypothesis postulates that the inflammatory immunological processes characteristic of the very first stages of atherosclerosis are initiated by humoral and cellular immune reactions against an endogenous antigen. Human Hsp60 expression itself is a response to injury initiated by several stress factors known to be risk factors for atherosclerosis, such as hypertension. Oxidized LDL is another candidate for an autoantigen in atherosclerosis. Antibodies to oxLDL have been detected in patients with atherosclerosis, and they have been found in atherosclerotic lesions. T lymphocytes isolated from human atherosclerotic lesions have been shown to respond to oxLDL and to be a major autoantigen in the cellular immune response. A third autoantigen proposed to be associated with atherosclerosis is 2-Glycoprotein I (2GPI), a glycoprotein that acts as an anticoagulant in vitro. 2GPI is found in atherosclerotic plaques, and hyper-immunization with 2GPI or transfer of 2GPI-reactive T cells enhances fatty streak formation in transgenic atherosclerotic-prone mice.

Infections may contribute to the development of atherosclerosis by inducing both inflammation and autoimmunity. A large number of studies have demonstrated a role of infectious agents, both viruses (cytomegalovirus, herpes simplex viruses, enteroviruses, hepatitis A) and bacteria (C. pneumoniae, H. pylori, periodontal pathogens) in atherosclerosis. Recently, a new “pathogen burden” hypothesis has been proposed, suggesting that multiple infectious agents contribute to atherosclerosis, and that the risk of cardiovascular disease posed by infection is related to the number of pathogens to which an individual has been exposed. Of single micro-organisms, C. pneumoniae probably has the strongest association with atherosclerosis.

These hypotheses are closely linked and not mutually exclusive. Modified LDL is cytotoxic to cultured endothelial cells and may induce endothelial injury, attract monocytes and macrophages, and stimulate smooth muscle growth. Modified LDL also inhibits macrophage mobility, so that once macrophages transform into foam cells in the subendothelial space they may become trapped. In addition, regenerating endothelial cells (after injury) are functionally impaired and increase the uptake of LDL from plasma.

Atherosclerosis is characteristically silent until critical stenosis, thrombosis, aneurysm, or embolus supervenes. Initially, symptoms and signs reflect an inability of blood flow to the affected tissue to increase with demand, e.g. angina on exertion, intermittent claudication. Symptoms and signs commonly develop gradually as the atheroma slowly encroaches on the vessel lumen. However, when a major artery is acutely occluded, the symptoms and signs may be dramatic.

As mentioned above, currently, due to lack of appropriate diagnostic strategies, the first clinical presentation of more than half of the patients with coronary artery disease is either myocardial infarction or death. Further progress in prevention and treatment depends on the development of strategies focused on the primary inflammatory process in the vascular wall, which is fundamental in the etiology of atherosclerotic disease. Without good surrogate markers that accurately report the activity and/or extent of vessel wall disease, methods cannot be developed that completely define risk, monitor the effects of risk reduction toward primary disease amelioration, or develop new classes of therapies that target the vessel wall.

One promising approach is the identification of circulating proteins that reflect the degree and character of vascular inflammation. A number of immune modulatory proteins have been identified to have some value as surrogate markers, but such biomarkers have not been shown to add sufficient information to have clinical utility. This is due to: i) the failure to consider data on multiple markers measured in parallel, ii) the failure to integrate individual marker data with clinical data that modulates the levels of circulating proteins and obscures the informative patterns, iii) inherited genetic variation that contributes to expression levels of the genes encoding the markers and confounds the abundance measurements, and iv) a lack of information regarding specific immune pathways activated in ASCVD that would better inform biomarker choice. Finally, the prior art fails to provide effective diagnostic or predictive methods using measurements of a panel of circulating proteins.

### Unmet Clinical and Scientific Need

Thus, there is an unmet need for use in clinical medicine and biomedical research for improved tools to identify individuals with vascular inflammation and atherosclerotic cardiovascular disease. At present, although insights into mechanisms and circumstances of atherosclerosis are increasing, our methods for identifying high-risk patients and predicting the efficacy of prevention strategies remain inadequate. New approaches therefore are needed to better diagnose patients at risk; identification of patients with atherosclerotic disease can lead to initiation of much needed therapy that can lead to improved clinical outcomes. The present invention addresses these and other shortcomings of the prior art.

### SUMMARY OF THE INVENTION

This invention provides methods for detection of circulating protein expression for diagnosis, monitoring, and development of therapeutics, with respect to atherosclerotic conditions, including but not limited to conditions that lead to angina, unstable angina, acute coronary syndrome, myocardial infarction, and heart failure. Specifically, circulating proteins are identified and described herein that are differentially expressed in atherosclerotic patients, including but not limited to circulating inflammatory markers. Circulating inflammatory markers identified herein include MCP-1, MCP-2, MCP-3, MCP-4, eotaxin, IP-10, M-CSF, IL-3, TNFa, Ang-2, IL-5, IL-7, and IGF-1.

The detection of circulating levels of proteins identified herein, which are specifically produced in the vascular wall as a result of the atherosclerotic process, can classify patients as belonging to atherosclerotic conditions, including atherosclerotic disease, no disease, myocardial infarction, stable angina, treatment with medication, no treatment, and the like. Such classification can also be used in prediction of cardiovascular events and response to therapeutics; and are useful to predict and assess complications of cardiovascular disease.

In one embodiment of the invention, the expression profile of a panel of proteins is evaluated for conditions indicative of various stages of atherosclerosis and clinical sequelae thereof. Such a panel provides a level of discrimination not found with individual markers. In one embodiment, the expression profile is determined by measurements of protein concentrations or amounts.

Methods of analysis may include, without limitation, utilizing a dataset to generate a predictive model, and inputting test sample data into such a model in order to classify the sample according to an atherosclerotic classification, where the classification is selected from the group consisting of an atherosclerotic disease classification, a healthy classification, a vascular inflammation classification, a medication exposure classification, a no medication exposure classification, and a coronary calcium score classification, and classifying the sample according to the output of the process. In some embodiments, such a predictive model is used in classifying a sample obtained from a mammalian subject by obtaining a dataset associated with a sample, wherein the dataset comprises at least three, or at least four, or at least five protein markers selected from the group consisting of MCP1; MCP2; MCP3; MCP4; Eotaxin; IP10; MCSF; IL3; TNFa; Ang2; IL5; IL7; IGF1; IL10; INFγ; VEGF; MIPla; RANTES; IL6; IL8; ICAM; TIMP1; CCL19; TCA4/6kine/CCL21; CSF3; TRANCE; IL2; IL4; IL13; Il1b; MCP5; CCL9; CXCL1/GRO1; GROalpha; IL12; and Leptin. The data optionally includes a profile for clinical indicia; additional protein expression profiles; metabolic measures, genetic information, and the like.

A predictive model of the invention utilizes quantitative data from one or more sets of markers described herein. In some embodiments a predictive model provides for a level of accuracy in classification; i.e. the model satisfies a desired quality threshold. A quality threshold of interest may provide for an accuracy or AUC of a given threshold, and either or both of these terms (AUC; accuracy) may be referred to herein as a quality metric. A predictive model may provide a quality metric, e.g. accuracy of classification or AUC, of at least about 0.7, at least about 0.8, at least about 0.9, or higher. Within such a model, parameters may be appropriately selected so as to provide for a desired balance of sensitivity and selectivity.

In other embodiments, analysis of circulating proteins is used in a method of screening biologically active agents for efficacy in the treatment of atherosclerosis. In such methods, cells associated with atherosclerosis, e.g. cells of the vessel wall, etc., are contacted in culture or in vivo with a candidate agent, and the effect on expression of one or more of the markers, e.g. a panel of markers, is determined. In another embodiment, analysis of differential expression of the above circulating proteins is used in a method of following therapeutic regimens in patients. In a single time point or a time course, measurements of expression of one or more of the markers, e.g. a panel of markers, is determined when a patient has been exposed to a therapy, which may include a drug, combination of drugs, non-pharmacologic intervention, and the like.

In another method, relative quantitative measures of 3 or more of atherosclerosis associated proteins identified herein are used to diagnose or monitor atherosclerotic disease in an individual. This panel of proteins identified herein can further include other clinical indicia; additional protein expression profiles; metabolic measures, genetic information, and the like.

In another embodiment, the invention includes methods for classifying a sample obtained from a mammalian subject by obtaining a dataset associated with a sample, wherein the dataset comprises quantitative data for at least three, or at least four, or at least five, or at least six, or at least seven, or at least eight, or at least nine, or more than nine protein markers selected from the group consisting of MCP-1, MCP-2, MCP-3, MCP-4, eotaxin, IP-10, M-CSF, IL-3, TNFa, Ang-2, IL-5, IL-7, and IGF-1, inputting the data into an analytical process that uses the data to classify the sample, where the classification is selected from the group consisting of an atherosclerotic disease classification, a healthy classification, a vascular inflammation classification, a medication exposure classification, a no medication exposure classification, and a coronary calcium score classification, and classifying the sample according to the output of the process.

In another embodiment, the invention includes methods for classifying a sample obtained from a mammalian subject by obtaining a dataset associated with a sample, wherein the dataset comprises quantitative data for at least three, or at least four, or at least five, or at least six, protein markers that each shows a correlation between a circulating protein concentration and an atherosclerotic vascular tissue RNA concentration, inputting the data into an analytical process that uses the data to classify the sample, where the classification is selected from the group consisting of an atherosclerotic disease classification, a healthy classification, a vascular inflammation classification, a medication exposure classification, a no medication exposure classification, and a coronary calcium score classification, and classifying the sample according to the output of the process.

### BRIEF DESCRIPTION OF THE SEVERAL VIEWS OF THE DRAWINGS

FIG. 1. Time-dependent serum inflammatory protein expression during progression of atherosclerosis in apolipoprotein (apo)E-deficient mice on high-fat diet. The heat map is a graphic representation of the serum concentration levels with individual serum samples arranged along the x-axis and protein markers along the y-axis. Values represent serum protein expression levels from apoe-deficient mice at baseline (T00; n=5) and at 10 (T10; n=5), 16 (T16; n=4), 24 (T24; n=5), and 40 wk (T40; n=5) on high-fat diet. Please note that for the 16-wk time point, values were derived from a 2nd independent data set.

FIG. 2. Circulating inflammatory protein expression levels in apoE-deficient mice and in control mice. Heat map is graphic representation of row normalized expression values. Values represent average circulating protein expression levels (log2) from replicate apoe-mice at baseline (T00)(n=9) and at 40 weeks (T40) on high fat diet (n=9), as well as C57B1/6 (n=5) and C3H/HeJ (n=3) mice at baseline and at 40 weeks on high fat diet (n=5, 5 respectively). Whereas apoE-deficient mice on high fat diet have the highest levels of inflammatory markers, C3H/HeJ mice have the lowest levels despite being on high fat diet as well. N-way ANOVA was used to identify with statistically significant variation among the various conditions. In far right column, p-values reported do not take into account possible interaction between diet, strain, and time. Effects of these factors and their interaction with each other are discussed in the text.

FIG. 3. Proteomic signature patterns of serum inflammatory markers in classification of atherosclerosis in mice. A: identification of the atherosclerosis classification protein subset. Various classification algorithms, including prediction analysis for microarrays (PAM), recursive feature elimination (RFE), support vector machine (SVM), and ANOVA, were used to rank a subset of markers based on their ability to accurately discriminate between mice with 4 different stages of atherosclerotic disease (apoE-deficient mice at baseline and 10, 24, and 40 wk on high-fat diet). A number of these markers were ranked in all classification algorithms. B: classification accuracy of mouse atherosclerotic disease (confusion matrix). To determine the accuracy of mouse classifier proteins in predicting disease severity, we used the top-ranking protein markers identified earlier (Ccl21, Ccl9, Csf3, Tnfsf11, Vegfa, Ccl11, Ccl2). The SVM algorithm was utilized for cross-validation of mouse experiments grouped on the basis of stages of disease. Accuracy of classification was determined with a 1,000-step N-fold cross-validation method, with 25% of experiments employed as the test group and the rest as the training group. Results are represented in tabular fashion with the confusion matrix as described in the Methods section. The notation “TRUE” refers to “Actual Disease State,” whereas “Predicted” refers to “Predicted Disease State.” C: classification of an independent data set. Using the SVM algorithm, we can classify an independent data set (“test”) to closest time point from the original set of experiments (“known”). The known experiments include the 4 time points in our original analysis from which the set of protein classifiers was derived. The independent set of experiments was derived from the 16-wk time point, which was not included in the original set. SVM scores (affinity) for each experiment, based on one-vs.-all comparisons, are represented graphically in the heat map. The protein profile of the 16-wk time point correlated more closely with the 10-wk time point of the original data set.

FIG. 4. Correlation between serum level and vascular gene expression of top classifier markers. A: to investigate the disease-related gene expression for a subset of these serum markers, we studied their temporal gene expression in aortas of mice from which the sera were obtained. Using quantitative real-time RT-PCR (qRT-PCR), we were able to correlate the time-dependent serum protein levels of these markers with their vascular wall gene expression. Pearson correlation was determined for log10-normalized average expression ratios of serum protein levels and aortic gene expression values. The average ratio of protein levels was determined by protein microarray at each time point divided by levels for apoE deficient mice at baseline (n=4-9). Average ratio of gene expression levels was determined by replicate qRT-PCR reaction at each time point divided by values obtained for apoe-deficient mice at baseline. Please note that, for the 16-wk time point, the values were derived from a separate independent data set. B: correlation matrix summary table for Pearson correlation values comparing normalized average ratios of serum protein level, vascular gene expression, and time on high-fat diet (log10 of no. of wk on diet). Correlations were considered significant at 0.05 (2 tailed).

FIG. 5. Clinical characteristics of the subjects. Nominal variables (*) are expressed as count (%), and continuous variables (†) as median (interquartiles range). ‡ Comparisons are made by Pearson Chi-square or Mann-Whitney U test, as appropriate. Significance has been calculated by Monte Carlo approach, based on 10000 sampled comparisons. BP (Blood Pressure); FH (Family History); ACEI (Angiotensin-Converting-Enzyme Inhibitors); BB (Beta Blockers); CCB (Calcium-Channel Blockers); AB (Alpha Blockers); ASA (Acetyl Salicylic Acid); BMI (Body Mass Index); DBP (Diastolic Blood Pressure); SBP (Systolic Blood Pressure); HR (Heart Rate); CRP (C-Reactive Protein).

FIG. 6. Serum chemokine profiles in coronary artery disease patients and healthy controls, before and after adjustment for clinical characteristics. Data are expressed as geometrical mean (95% CI). Adjustment has been performed by GLM multivariate analysis and comparisons on adjusted means by t-test. * Model 1 is adjusted for age and waist circumference; † Model 2 is adjusted as Model 1 plus treatment (ACE inhibitors, statins, and aspirin).

FIG. 7. Two dimensional hierarchical clustering of clinical variables and cases versus controls.

FIG. 8. Principal component analysis demonstrating that 60-70% of the variability observed within the subjects could be explained by chemokines, insulin resistance profile, and a subset of other clinical variables such as hypertension and hyperlipidemia, with markers of inflammation being the dominant factor.

FIG. 9. Table showing Support Vector Machine (SVM) and Recursive Feature Elimination (RFE) used to determine optimal number of ranked variables to classify experiments into correct groups at minimal error rate. Optimal error rate or misclassification is calculated by 1000-times reiterated cross-validation, with 25% of experiments as test group and remaining experiments as training group.

FIG. 10. ROC curves.

FIG. 11. Table showing Logistic Regression models to predict coronary artery disease. Models: 1) Stepwise forward selection without missing values estimation; 2) Stepwise forward selection with missing data estimation by conditional means; 3) Stepwise forward selection of clinical variables and chemokine score. Independent variables: Age, Gender, Diastolic blood pressure (DBP), Systolic blood pressure (SBP), Heart rate, Plasma insulin, C-Reactive Protein, and chemokines (models 1 and 2: Eotaxin, IP-10, MCP-1, MCP-2, MCP-3, MCP-4, and MIP-1alpha (; model 3: Chemokine score).

FIG. 12. Expected AUC value and S.E. for a series of LDA models involving an increasing number of terms in the order given in the figure.

FIG. 13. Expected AUC value and S.E. for a series of Logistic Regression models involving an increasing number of terms in the order given in the figure.

FIG. 14. LDA model predictions with MCP-1 marker excluded from the set of available predictive markers. The new model utilizes Ang-2, IGF-1 and M-CSF as alternate marker combination for exceeding the AUC>0.75 threshold.

FIG. 15a. Marker selection for a Logistic Regression model using Akaike Information Criterion (AIC).

FIG. 15b: Expected AUC value and S.E. for a series of Logistic Regression models involving an increasing number of terms in the order given in the figure (=inverse order of term removal from the complete model by applying the AIC criterion in the marker selection process).

FIG. 16. Logistic regression model including both clinical variables and biological markers.

FIG. 17. Logistic regression model including alternate clinical variables and biological markers. A model including “Beta Blockers” (DC512) and “Statins” (DC3005) and MCP-4 produces an expected value of AUC in excess of 0.85.

FIG. 18. Boxplots of value distribution of the first discriminant variate for the three groups: “Untreated,” “ACE or Statins,” and “ACE and Statins.”

### DETAILED DESCRIPTION OF THE INVENTION

Definitions

Terms used in the claims and specification are defined as set forth below unless otherwise specified.

The term “ameliorating” refers to any therapeutically beneficial result in the treatment of a disease state, e.g., an atherosclerotic disease state, including prophylaxis, lessening in the severity or progression, remission, or cure thereof.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel, F M, et al., Current Protocols in Molecular Biology, 4, John Wiley & Sons, Inc., Brooklyn, N.Y., A.1E.1-A.1F.11, 1996-2004).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to alter a protein expression profile.

The term “therapeutically effective amount” is an amount that is effective to ameliorate a symptom of a disease. A therapeutically effective amount can be a “prophylactically effective amount” as prophylaxis can be considered therapy.

TP: true positive

TN: true negative

FP: false positive

FN: false negative

N: total number of negative samples

P: total number of positive samples

A: total number of samples

Accuracy=(TP+TN)/A

Mean CV error=Mean Misclassification error=1−Mean Accuracy

Sensitivity=TP/P=TP/(TP+FN)

Specificity=TN/N=TN/(TN+FP)

Abbreviations used in this application include the following: CAD=coronary artery disease; MIP1a=MIP1alpha; LDA=Linear Discriminant Analysis, MI=myocardial infarction; ASCVD=atherosclerotic cardiovascular disease.

It must be noted that, as used in the specification and the appended claims, the singular forms “a,” “an,” and “the” include plural referents unless the context clearly dictates otherwise.

Atherosclerosis (also referred to as arteriosclerosis, atheromatous vascular disease, arterial occlusive disease) as used herein, refers to a cardiovascular disease characterized by plaque accumulation on vessel walls and vascular inflammation. The plaque consists of accumulated intracellular and extracellular lipids, smooth muscle cells, connective tissue, inflammatory cells, and glycosaminoglycans. Inflammation occurs in combination with lipid accumulation in the vessel wall, and vascular inflammation is with the hallmark of atherosclerosis disease process.

Myocardial infarction is an ischemic myocardial necrosis usually resulting from abrupt reduction in coronary blood flow to a segment of myocardium. In the great majority of patients with acute MI, an acute thrombus, often associated with plaque rupture, occludes the artery that supplies the damaged area. Plaque rupture occurs generally in previously partially obstructed by an atherosclerotic plaque enriched in inflammatory cells. Altered platelet function induced by endothelial dysfunction and vascular inflammation in the atherosclerotic plaque presumably contributes to thrombogenesis. Myocardial infarction can be classified into ST-elevation and non-ST elevation MI (also referred to as unstable angina). In both forms of myocardial infarction, there is myocardial necrosis. In ST-elevation myocardial infraction there is transmural myocardial injury which leads to ST-elevations on electrocardiogram. In non-ST elevation myocardial infarction, the injury is sub-endocardial and is not associated with ST segment elevation on electrocardiogram. Myocardial infarction (both ST and non-ST elevation) represents an unstable form of atherosclerotic cardiovascular disease. Acute coronary syndrome encompasses all forms of unstable coronary artery disease.

Angina refers to chest pain or discomfort resulting from inadequate blood flow to the heart. Angina can be a symptom of atherosclerotic cardiovascular disease. Angina may be classified as stable, which follows a regular chronic pattern of symptoms. Unlike the unstable forms of atherosclerotic vascular disease. The pathophysiological basis of stable atherosclerotic cardiovascular disease is also complicated but is biologically distinct from the unstable form. Generally stable angina is not myocardial necrosis.

Heart failure can occur as a result of myocardial dysfunction caused by myocardial infraction.

Several features of the current approach should be noted. Atherosclerosis and related conditions are diagnosed through a blood based test that assesses the presence of one or a panel of protein markers. The markers include MCP-1, MCP-2, MCP-3, MCP-4, eotaxin, IP-10, M-CSF, IL-3, TNFa, Ang-2, IL-5, IL-7, and IGF-1. These markers have been shown to be specifically produced in the vascular wall in association with the atherosclerotic process. In some embodiments, such a predictive model utilizes quantitative data obtained from circulating markers that include MCP1; MCP2; MCP3; MCP4; Eotaxin; IP10; MCSF; IL3; TNFa; Ang2; IL5; IL7; IGF1; IL10; INFγ; VEGF; MIP1a; RANTES; IL6; IL8; ICAM; TIMP1; CCL19; TCA4/6kine/CCL21; CSF3; TRANCE; IL2; IL4; IL13; Il1b; MCP5; CCL9; CXCL1/GRO1; GROalpha; IL12; and Leptin. Other circulating markers of interest include sVCAM; sICAM-1; E-selectin; P-selection; interleukin-6, interleukin-18; creatine kinase; LDL, oxLDL, LDL particle size, Lipoprotein(a); troponin I, troponin T; LPLA2; CRP; HDL, Triglyceride, insulin, BNP (brain naturetic peptide), fractalkine, osteopontin, osteoprotegerin, oncostatin-M, Myeloperoxidase, ADMA, PAI-1 (plasminogen activator inhibitor), SAA (circulating amyloid A), t-PA (tissue-type plasminogen activator), sCD40 ligand, fibrinogen, homocysteine, D-dimer, leukocyte count and may further include a variety of additional markers as described herein, including clinical indicia, metabolic measures, genetic assays, and additional circulating markers.

In certain embodiments of the invention, a dataset for classification is obtained from a patient sample, wherein the dataset comprises quantitative data for at least three protein markers selected from the group consisting of MCP-1, MCP-2, MCP-3, MCP-4, eotaxin, IP-10, M-CSF, IL-3, TNFa, Ang-2, IL-5, IL-7, and IGF-1. The at least three protein markers may comprise a marker set selected from the group consisting of MCP-1, IGF-1, TNFa; MCP-1, IGF-1, M-CSF; ANG-2, IGF-1, M-CSF; and MCP-4, IGF-1, M-CSF. Where the dataset comprises quantitative data from at least four protein markers, the at least four protein markers may be selected from the group consisting of MCP-1, MCP-2, MCP-3, MCP-4, eotaxin, IP-10, M-CSF, IL-3, TNFa, Ang-2, IL-5, IL-7, and IGF-1; MCP-1, IGF-1, TNFa, IL-5; MCP-1, IGF-1, M-CSF, MCP-2; ANG-2, IGF-1, M-CSF, IL-5; MCP-1, IGF-1, TNFa, MCP-2; and MCP-4, IGF-1, M-CSF, IL-5. Where the dataset comprises quantitative data from at least five markers, The at least five markers may comprise a marker set selected from the group consisting of MCP-1, MCP-2, MCP-3, MCP-4, eotaxin, IP-10, M-CSF, IL-3, TNFa, Ang-2, IL-5, IL-7, and IGF-1; MCP-1, IGF-1, TNFa, IL-5, M-CSF; MCP-1, IGF-1, M-CSF, MCP-2, IP-10; ANG-2, IGF-1, M-CSF, IL-5, TNFa; MCP-1, IGF-1, TNFa, MCP-2, IP-10; MCP-4, IGF-1, M-CSF, IL-5, TNFa; and MCP-4, IGF-1, M-CSF, IL-5, MCP-2.

In another embodiment of the invention, at least two, at least three, at least four, at least five or more markers are selected from M-CSF, eotaxin, IP-10, MCP-1, MCP-2, MCP-3, MCP-4, IL-3, IL-5, IL-7, IL-8, MIP1a, TNFa, and RANTES.

The identification of atherosclerosis associated circulating proteins provides diagnostic and prognostic methods, which detect the occurrence of a disorder, e.g. coronary arterial disease, atherosclerosis, etc., particularly where such a disorder is indicative of a propensity for myocardial infarction, heart failure, etc.; or assess an individual's susceptibility to such disease, by detecting altered levels of the identified circulating proteins. The methods also include screening for efficacy of therapeutic agents and methods; disease staging and classification; and the like. Early detection can be used to determine the occurrence of developing disease, thereby allowing for intervention with appropriate preventive or protective measures.

TABLE 1

| | | | | | | | | | |
|---|---|---|---|---|---|---|---|---|---|
| | | | | Human | | | | | |
| | | | | polynucleotide | Human | Mouse | Mouse | | Mouse |
| | | | Locus | accession | polynucleotide | polynucleotide | polynucleotide | Human protein | protein |
| Protein | Common Alias | Other names | Link | (refseq) | accession (related) | accession (refseq) | accession (related) | accession | accession |

TABLE 1A

| | | | | | | | | | |
|---|---|---|---|---|---|---|---|---|---|
| CCL2 | \|\|CCL2\|\|SCYA2\|\|MCP1\|\|MONOCYTE | Chemokine(C—C | 6347 | NM_002982 | AC005549, | NM_011333 | AJ238892, | NP_002973, | NP_035463, |
| | CHEMOTACTIC | motif) ligand 2 | | (SEQ ID NO: 1) | AF519531, | (SEQ ID NO: 2) | AL626807, J04467, | P13500, | P10148, |
| | PROTEIN 1\|\|SMALL | | | | AY357296, D26087, | | M19681, | Q6UZ82 | Q5SVU3 |
| | INDUCIBLE CYTOKINE | | | | M28225, M31626, | | CB571537, | (SEQ ID NOS | (SEQ ID |
| | A2\|\|chemokine (C—C motif) | | | | M37719, X60001, | | AF065929, | 3-5) | NOS 6-8) |
| | ligand 2\|\|MONOCYTE | | | | Y18933, AV733621, | | AF065930, | | |
| | CHEMOTACTIC AND | | | | BC009716, | | AF065931, | | |
| | ACTIVATING | | | | BG530064, | | AF065932, | | |
| | FACTOR\|\|CHEMOKINE, | | | | BT007329, M24545, | | AF065933, | | |
| | CC MOTIF, LIGAND | | | | M26683, M28226, | | AK132590, | | |
| | 2\|\|MCAF CORONARY | | | | S69738, S71513, | | AK150937, | | |
| | ARTERY DISEASE, | | | | X14768, BU570769, | | AK151789, | | |
| | MODIFIER | | | | | | AK153443, | | |
| | OF\|\|CORONARY | | | | | | AK153468, | | |
| | ARTERY DISEASE, | | | | | | AK153520, | | |
| | DEVELOPMENT OF, IN | | | | | | BC055070, | | |
| | HIV\|\| | | | | | | CT010187, J04467 | | |
| CCL8 | \|\|CCL8\|\|MCP2\|\|SCYA8\|\|MONOCYTE | Chemokine (C—C | 6355 | NM_005623 | AC011193, X99886, | NM_021443 | AL713860, | NP_005614, | NP_067418, |
| | CHEMOTACTIC | motif) ligand 8 | | (SEQ ID NO: 9) | Y18047, Y16645, | (SEQ ID NO: 10) | AK007942, | P80075 | Q5SR19, |
| | PROTEIN 2\|\|chemokine (C—C | | | | Y10802 | | AB023418, | (SEQ ID NOS | Q9Z121 |
| | motif) ligand | | | | | | AI604201 | 11-12) | (SEQ ID |
| | 8\|\|CHEMOKINE, CC | | | | | | | | NOS 13-15) |
| | MOTIF, LIGAND | | | | | | | | |
| | 8\|\|SMALL INDUCIBLE | | | | | | | | |
| | CYTOKINE SUBFAMILY | | | | | | | | |
| | A, MEMBER 8\|\| | | | | | | | | |
| CCL7 | \|\|SCYA7\|\|CCL7\|\|MCP3\|\|MONOCYTE | Chemokine (C—C | 6354 | NM_006273 | AC005549, X72309, | NM_013654 | AL626807, | NP_006264, | NP_038682, |
| | CHEMOTACTIC | motif) ligand 7 | | (SEQ ID NO: | CA306760, | (SEQ ID NO: 17) | AL645596, | P80098, | Q03366, |
| | PROTEIN 3\|\|SMALL | | | 16) | AF043338, | | X70058, BF142314, | Q569J6, | Q5SVU0 |
| | INDUCIBLE CYTOKINE | | | | BC070240, | | AF128193, | Q7Z7Q8 | (SEQ ID |
| | A7\|\|chemokine (C—C motif) | | | | BC09235, | | AF128194, | (SEQ ID NOS | NOS 22-24) |
| | ligand 7\|\|CHEMOKINE, | | | | BC112258, | | AK078824, | 18-21) | |
| | CC MOTIF, LIGAND 7\|\| | | | | BC112260, X71087 | | BC061126, L04694, | | |
| | | | | | | | S71251, Z12297 | | |
| CCL13 | \|\|NCC1\|\|SCYA13\|\|MCP4\|\|CCL13\|\| | Chemokine (C—C | 6357 | NM_005408 | AC002482, | NM_010779 | AC163646, | NP_005399 | P21812 |
| | NEW CC | motif) ligand 13 | | (SEQ ID NO: | AC011193, | (SEQ ID NO: 26) | M55616, | (SEQ ID NO: | (SEQ ID |
| | CHEMOKINE | | | 25) | AJ000979, | | AB051900, | 27) | NO: 28) |
| | 1\|\|MONOCYTE | | | | AJ001634, | | AK144385, | | |
| | CHEMOTACTIC | | | | BC008621, | | AY007569, | | |
| | PROTEIN 4\|\|chemokine (C—C | | | | BT007385, | | BC026198, | | |
| | motif) ligand | | | | CR450337, U46767, | | M55617, X68804 | | |
| | 13\|\|CHEMOKINE, CC | | | | U59808, X98306, | | | | |
| | MOTIF, LIGAND | | | | Z77650, Z77651, | | | | |
| | 13\|\|SMALL INDUCIBLE | | | | U59808, BM991948 | | | | |
| | CYTOKINE SUBFAMILY | | | | | | | | |
| | A, MEMBER 13\|\| | | | | | | | | |
| CCL11 | \|\|SCYA11\|\|CCL11\|\|EOTAXIN\|\| | Chemokine (C—C | 6356 | NM_002986 | AB063614, | NM_011330 | AL645596, | NP_002977, | NP_035460, |
| | SMALL INDUCIBLE | motif) ligand 11 | | (SEQ ID NO: | AB063616, | (SEQ ID NO: 30) | U77462, | P51671, | P48298, |
| | CYTOKINE | | | 29) | AC005549, U34780, | | AF128205, | Q6I9T4 | Q5SVB5 |
| | A11\|\|CHEMOKINE, CC | | | | U46572, Z92709, | | AF128206, | (SEQ ID NOS | (SEQ ID |
| | MOTIF, LIGAND | | | | BC017850, | | AF128207, | 31-33) | NOS 34-36) |
| | 11\|\|chemokine (C—C motif) | | | | BF197516, | | AF128208, | | |
| | ligand 11\|\|SMALL | | | | CR457421, D49372, | | AF128209, | | |
| | INDUCIBLE CYTOKINE | | | | U46573, Z69291, | | AK009307, | | |
| | SUBFAMILY A, | | | | Z75668, Z75669, | | AK010146, | | |
| | MEMBER 11\|\| | | | | BG485598 | | BC027521, | | |
| | | | | | | | U26426, U40672, | | |
| | | | | | | | AA711712, | | |
| | | | | | | | Mm4686 | | |
| CXCL10 | \|\|INP10\|\|CXCL10\|\|SCYB10\|\| | Chemokine | 3627 | NM_001565 | AC112719, | NM_021274 | AC109603, | NP_001556, | NP_067249, |
| | IP10\|\|INTERFERON- | (C—X—C motif) | | (SEQ ID NO: | BC021117, M27087, | (SEQ ID NO: 38) | AC122365, L07417, | P02778 | P17515, |
| | GAMMA-INDUCED | ligand 10 | | 37) | M37435, M64592, | | M86830, | (SEQ ID NOS | Q548V9 |
| | FACTOR\|\|INTERFERON- | | | | M76453, U22386, | | AF227743, | 39-40) | (SEQ ID |
| | GAMMA-INDUCIBLE | | | | X05825, BC010954, | | AJ243095, | | NOS 41-43) |
| | PROTEIN 10\|\|MOB1, | | | | X02530 | | AK144279, | | |
| | MOUSE, HOMOLOG | | | | | | AK146144, | | |
| | OF\|\|CHEMOKINE, CXC | | | | | | AK150380, | | |
| | MOTIF, LIGAND | | | | | | AK150765, | | |
| | 10\|\|chemokine (C—X—C | | | | | | AK150987, | | |
| | motif) ligand 10\|\|SMALL | | | | | | AK151210, | | |
| | INDUCIBLE CYTOKINE | | | | | | AK151248, | | |
| | SUBFAMILY B, | | | | | | AK151415, | | |
| | MEMBER 10\|\| | | | | | | AK151534, | | |
| | | | | | | | AK152234, | | |
| | | | | | | | AK152568, | | |
| | | | | | | | AK152814, | | |
| | | | | | | | AK152838, | | |
| | | | | | | | AK152924, | | |
| | | | | | | | AK153181, | | |
| | | | | | | | AK156907, | | |
| | | | | | | | AK157130, | | |
| | | | | | | | AK157139, | | |
| | | | | | | | AK157589, | | |
| | | | | | | | AK157678, | | |
| | | | | | | | AK172540, | | |
| | | | | | | | BC030067, | | |
| | | | | | | | BC057150, | | |
| | | | | | | | M33266, M86829, | | |
| | | | | | | | BC057150 | | |
| CSF1 | \|\|CSF1\|\|MCSF\|\|MGC31930\|\| | Colony | 1435 | NM_000757, | AL450468, M11038, | NM_007778 | AC140786, | NP_00748, | NP_031804, |
| | COLONY-STIMULATING | stimulating factor | | NM_172210, | M11295, M11296, | (SEQ ID NO: 48) | M81316, AI323836, | NP757349, | P07141 |
| | FACTOR 1\|\|COLONY- | 1 (macrophage) | | NM_172211, | X06106, BC021117, | | AK136808, | NP757350, | (SEQ ID |
| | STIMULATING FACTOR, | | | NM172212 | M27087, M37435, | | AK138489, | NP757351, | NOS 57-58) |
| | MACROPHAGE- | | | (SEQ ID NOS | M64592, M76453, | | AK154261, | P09603, | |
| | SPECIFIC\|\|macrophage | | | 44-47) | U22386, X05825, | | AK154872, | Q5VVF2, | |
| | colony stimulating | | | | BC021117 | | AK160995, | Q5VVF3, | |
| | factor\|\|Colony stimulating | | | | | | AK166370, | Q5VVF4 | |
| | factor 1 | | | | | | AK170154, | (SEQ ID NOS | |
| | (macrophage)\|\|colony | | | | | | BC025593, | 49-56) | |
| | stimulating factor 1 isoform | | | | | | BC066187, | | |
| | a precursor\|\|colony | | | | | | BC066200, | | |
| | stimulating factor 1 isoform | | | | | | BC066205, | | |
| | c precursor\|\|colony | | | | | | BG067715, | | |
| | stimulating factor 1 isoform | | | | | | BG080688, | | |
| | b precursor\|\| | | | | | | M15692, M21149, | | |
| | | | | | | | M21952, S78392, | | |
| | | | | | | | X05010, M21952 | | |
| IL3 | \|\|IL3\|\|MULTI- | Interleukin 3 | 3562 | NM_000588 | AC004511, | NM_010556 | AL596103, | NP_000579, | P01586, |
| | CSF\|\|Interleukin 3 (colony- | (colony- | | (SEQ ID NO: | AC034228, | (SEQ ID NO: 60) | K03233, M14394, | P08700, | K01850, |
| | stimulating factor, | stimulating | | 59) | AF365976, | | M20128, X02732, | Q6GS87, | Q5X77 |
| | multiple)\|\| | factor, multiple) | | | BC066272, | | AK153634, | Q6NZ78, | (SEQ ID |
| | | | | | BC066273, | | K01668, K01850, | Q6NZ79 | NOS 66-68) |
| | | | | | BC066274, | | A02046 | (SEQ ID NOS | |
| | | | | | BC066275, | | | 61-65) | |
| | | | | | BC066276, | | | | |
| | | | | | BC069472, M14743, | | | | |
| | | | | | M17115, M20137 | | | | |
| TNF | \|\|CACHECTIN\|\|TNFA\|\|TNF\|\| | Tumor necrosis | 7124 | NM_000594 | AB088112, | NM_013693 | AB039224, | NP_000585, | NP_038721, |
| | TNF, MACROPHAGE- | factor (TNF | | (SEQ ID NO: | AB202113, | (SEQ ID NO: 70) | AB039225, | P01375, | P06804 |
| | DERIVED\|\|TNF, | superfamily, | | 69) | AF129756, | | AB039226, | Q5RT83, | (SEQ ID |
| | MONOCYTE- | member 2) | | | AJ249755, | | AB039227, | Q5STB3, | NOS 76-77) |
| | DERIVED\|\|TUMOR | | | | AJ270944, | | AB039228, | Q9UBM5 | |
| | NECROSIS FACTOR, | | | | AL662801, | | AB039229, | (SEQ ID NOS | |
| | ALPHA\|\|tumor necrosis | | | | AL662847, | | AB039230, | 71-75) | |
| | factor (TNF superfamily, | | | | AL929587, | | AB039231, | | |
| | member 2)\|\| | | | | AY066019, | | AB039232, | | |
| | | | | | AY214167, | | AF109719, | | |
| | | | | | AY799806, | | CR974444, | | |
| | | | | | BA000025, | | D84196, D84199, | | |
| | | | | | BX248519, M16441, | | L22359, L22360, | | |
| | | | | | M26331, X02910, | | L22361, L22362, | | |
| | | | | | Y14768, Z15026, | | L22363, L22364, | | |
| | | | | | AF043342, | | L22365, M20155, | | |
| | | | | | AF098751, | | M38296, U06950, | | |
| | | | | | AJ227911, | | U68414, Y00467, | | |
| | | | | | AJ251878, | | AK153319, | | |
| | | | | | AJ251879, | | AK153800, | | |
| | | | | | BC028148, | | AK154223, | | |
| | | | | | BI908079, M10988, | | AK155964, | | |
| | | | | | M35592, X01394, | | AY423855, | | |
| | | | | | AF043342, BC028148, | | M11731, M13049, | | |
| | | | | | M10988, X01394 | | X02611, | | |
| ANGPT2 | \|\|ANG2\|\|angiopoietin- | Angiopoietin 2 | 285 | NM_001147 | AC018398, | NM_007426 | AC122206, | NP_001138, | NP_031452, |
| | 2B\|\|Tie2- | | | (SEQ ID NO: | AY563557, | (SEQ ID NO: 79) | AC129567, | O15123, | O35608 |
| | ligand\|\|ANGPT2\|\|AGPT2\|\|angiopoietin- | | | 78) | AB009865, | | AF004326, | Q9H4C0, | (SEQ ID |
| | 2a\|\|Angiopoietin 2\|\| | | | | AF004327, | | AK019860, | Q9H4C1, | NOS 85-86) |
| | | | | | AF187858, | | AK048622, | Q9HBP3 | |
| | | | | | AF218015, | | AK143974, | (SEQ ID NOS | |
| | | | | | AJ289780, | | AK156132, | 80-84) | |
| | | | | | AJ289781, | | AK186615, | | |
| | | | | | AK075219, | | BC027216 | | |
| | | | | | BC022490, | | | | |
| | | | | | CR620685 | | | | |
| IL5 | \|\|EDF\|\|IL5\|\|EOSINOPHIL | Interleukin 5 | 3567 | NM_000879 | AC116366, | NM_010558 | AC084392, | NP_000870, | NP_034688, |
| | DIFFERENTIATION | (colony- | | (SEQ ID NO: | AF353265, J02971, | (SEQ ID NO: 88) | AL645741, | P05113 | P04401, |
| | FACTOR\|\|Interleukin 5 | stimulating | | 87) | J03478, X12706, | | D14461, X04601, | (SEQ ID NOS | Q5SV01 |
| | (colony-stimulating factor, | factor, | | | BC066279, | | X06270 | 89-90) | (SEQ ID |
| | eosinophil)\|\| | eosinophil) | | | BC066280, | | | | NOS 91-93) |
| | | | | | BC066281, | | | | |
| | | | | | BC066282, | | | | |
| | | | | | BC069137, X04688, | | | | |
| | | | | | X12705 | | | | |
| IL7 | \|\|IL7\|\|Interleukin 7\|\| | Interleukin 7 | 3574 | NM_000880 | AC083837, M29053, | NM_008371 | AC125373, | NP_000871, | NP_032397, |
| | | | | (SEQ ID NO: | AB102879, | (SEQ ID NO: 95) | M29054, M29055, | P13232, | P10168, |
| | | | | 94) | AB102880, | | M29056, M29057, | Q5FBX5, | Q544C8, |
| | | | | | AB102882, | | AK040399, | Q5FBY5, | Q8C9S3 |
| | | | | | AB102883, | | AK041307, | Q5FBY6, | (SEQ ID |
| | | | | | AB102893, | | AK041403, | Q5FBY8, | NOS 103-106) |
| | | | | | AU136355, | | AK052452, | Q5FBY9 | |
| | | | | | BC032487, | | AK139858, | (SEQ ID NOS | |
| | | | | | BC047698, J04156, | | AK145184, | 96-102) | |
| | | | | | | | BC110553, | | |
| | | | | | | | BG069762, | | |
| | | | | | | | BG082754, X07962 | | |
| IGF1 | \|\|IGF1\|\|IGF 1\|\|INSULIN- | Insulin-like | 3479 | NM_000618 | AC010202, | NM_010512, | AC125082, | NP_000609, | NP_034642, |
| | LIKE GROWTH FACTOR | growth factor 1 | | (SEQ ID NO: | AY260957, | NM_184052 | AC139754, | P01343, | NP_908941, |
| | 1\|\|insulin-like growth factor | (somatomedin C) | | 107) | AY790940, M12659, | (SEQ ID NOS | M14983, M28139, | P05019, | P05017, |
| | 1 (somatomedin C)\|\| | | | | M14155, M14156, | 108-109) | AF440694, | Q13429, | Q4VjB9, |
| | | | | | S85346, X03420, | | AK038119, | Q14620, | Q4VJC0, |
| | | | | | X03421, X03422, | | AK050118, | Q59GC5, | Q547V2 |
| | | | | | X03563, AB209184, | | AK052033, | Q5U743, | (SEQ ID |
| | | | | | CR541861, M11568, | | AK081019, | Q6LD41, | NOS 120-125) |
| | | | | | M27544, M29644, | | AK155435, | Q9NP10, | |
| | | | | | M37484, U40870, | | AK165471, | Q9UC01 | |
| | | | | | X00173, X56773, | | AY878192, | (SEQ ID NOS | |
| | | | | | X56774, X57025 | | AY878193, | 110-119) | |
| | | | | | | | BC012409, | | |
| | | | | | | | BG071465, | | |
| | | | | | | | CT010364, | | |
| | | | | | | | X04480, X04482 | | |

TABLE 1B

| | | | | | | | | | |
|---|---|---|---|---|---|---|---|---|---|
| IL10 | \|\|IL10\|\|CSIF\|\|Interleukin | Interleukin 10 | 3586 | NM_000572 | AF295024, | NM_010548 | AL513351, | NP_000563, | NP_034678, |
| | 10\|\|CYTOKINE SYNTHESIS | | | (SEQ ID NO: | AF418271, | (SEQ ID NO: | M84340, | P22301, | P18893 |
| | INHIBITORY FACTOR\|\| | | | 126) | AL513315, | 127) | AK152344, | Q6FGS9, | (SEQ ID |
| | | | | | DQ217938, U16720, | | M37897 | Q6FGW4, | NOS 135-136) |
| | | | | | X78437, AF043333, | | | Q6LBF4, | |
| | | | | | AY029171, | | | Q71UZ1, | |
| | | | | | BC022315, | | | Q9BXR7 | |
| | | | | | BC104252, | | | (SEQ ID NOS | |
| | | | | | BC104253, | | | 128-134) | |
| | | | | | CR541993, | | | | |
| | | | | | CR542028, M57627 | | | | |
| IFNG | \|\|IFNG\|\|IFG\|\|IFI\|\|Interferon, | Interferon, | 3458 | NM_000619 | AC007458, | NM_008337 | AC153498, | NP_000610, | NP_032363, |
| | gamma\|\|IFN, IMMUNE\|\| | gamma | | (SEQ ID NO: | AF375790, J00219, | (SEQ ID NO: | AK089574, | P01579, | Q542B8, |
| | | | | 137) | AF506749, | 138) | AY423847, | Q14609, | P01580 |
| | | | | | AY044154, | | K00083, M28621 | Q14610, | (SEQ ID |
| | | | | | AY255837, | | | Q14611, | NOS 151-153) |
| | | | | | AY255839, | | | Q14612, | |
| | | | | | BC070256, V00543, | | | Q14613, | |
| | | | | | X01992, X13274, | | | Q14614, | |
| | | | | | X62468, X62469, | | | Q14615, | |
| | | | | | X62470, X62471, | | | Q53ZV4, | |
| | | | | | X62472, X62473, | | | Q8NHY9, | |
| | | | | | X62474, X87308 | | | Q96LA2 | |
| | | | | | | | | (SEQ ID NOS | |
| | | | | | | | | 139-150) | |
| VEGF | \|\|VEGF\|\|Vascular endothelial | Vascular | 7422 | NM_001025366, | AF095785, | NM_001025250, | AB086118, | NP_003367, | NP_001020421, |
| | growth factor\|\|VEGFA | endothelial | | NM_001025367, | AF437895, | NM_001025257, | AC127690, | NP_001020537, | NP001020428, |
| | ATHEROSCLEROSIS, | growth factor | | NM_001025368, | AL136131, M63978, | NM_009505 | AF317892, | NP_001020538, | NP033531, |
| | SUSCEPTIBILITY TO\|\| | | | NM_001025369, | S85224, AB021221, | (SEQ ID NOS | U41383, | NP_001020539, | Q00731, |
| | | | | NM_001025370, | AB209485, | 161-163) | AA959550, | NP_001020540, | Q5UD54 |
| | | | | NM_001033756, | AF022375, | | AI606078, | NP_001020541, | (SEQ ID |
| | | | | NM_003376 | AF024710, | | AK031905, | NP001028928, | NOS 177-181) |
| | | | | (SEQ ID NOS | AF062645, | | AK131850, | P15692, | |
| | | | | 154-160) | AF091352, | | AW913188, | Q59FH5, | |
| | | | | | AF214570, | | AY120866, | Q6WZM0, | |
| | | | | | AF323587, | | AY263146, | Q71S09, | |
| | | | | | AF430806, | | AY707864, | Q96FD9, | |
| | | | | | AF486837, | | AY750956, | Q9UNS8 | |
| | | | | | AJ010438, | | AY750957, | (SEQ ID NOS | |
| | | | | | AK056914, | | AY756068, | 164-176) | |
| | | | | | AK125666, | | BC022642, | | |
| | | | | | AY047581, | | BC061468, | | |
| | | | | | AY263145, | | BQ554097, | | |
| | | | | | AY500353, | | BQ832724, | | |
| | | | | | AY766116, | | CA321456, | | |
| | | | | | BC011177, | | M95200, S37052, | | |
| | | | | | BC019867, | | S38083, S38100, | | |
| | | | | | BC058855, | | U50279 | | |
| | | | | | BC065522, | | | | |
| | | | | | BQ880667, | | | | |
| | | | | | BU153227, | | | | |
| | | | | | CN256173, | | | | |
| | | | | | CR614384, | | | | |
| | | | | | CX756573, M27281, | | | | |
| | | | | | M32977, S85192, | | | | |
| | | | | | X62568 | | | | |
| CCL3 | \|\|SCYA3\|\|CCL3\|\|MIP1A\|\|LD7 | Chemokine (C—C | 6348 | NM_002983 | AC069363, D90144, | NM_011337 | AL596122, | NP_002974, | NP_035467, |
| | 8-ALPHA\|\|MACROPHAGE | motif) ligand 3 | | (SEQ ID NO: | M23178, X04018, | (SEQ ID NO: | M73061, X53372, | P10147, | P10855, |
| | INFLAMMATORY | | | 182) | AF043339, | 183) | AF065939, | Q14745 | Q5QNW0 |
| | PROTEIN 1- | | | | BC071834, D00044, | | AF065940, | (SEQ ID NOS | (SEQ ID |
| | ALPHA\|\|SMALL | | | | D63785, M23452, | | AF065941 | 184-186) | NOS 187-189) |
| | INDUCIBLE CYTOKINE | | | | M25315, X03754, | | AF065942, | | |
| | A3\|\|chemokine (C—C motif) | | | | CR591007 | | AF065943, | | |
| | ligand 3\|\|CHEMOKINE, CC | | | | | | AK150590, | | |
| | MOTIF, LIGAND 3\|\| | | | | | | AK150634, | | |
| | | | | | | | AK150698, | | |
| | | | | | | | AK151581, | | |
| | | | | | | | AK152648, | | |
| | | | | | | | AK153155, | | |
| | | | | | | | AK155058, | | |
| | | | | | | | J04491, M23447, | | |
| | | | | | | | X12531, | | |
| | | | | | | | AA895994 | | |
| CCL5 | \|\|TCP228\|\|SCYA5\|\|CCL5\|\|T | Chemokine (C—C | 6352 | NM_002985 | AB023652, | NM_013653 | AB051897, | NP_002976, | NP_038681, |
| | CELL-SPECIFIC RANTES\|\|T | motif) ligand 5 | | (SEQ ID NO: | AB023653, | (SEQ ID NO: | AL596122, | P13501, | P30882, |
| | CELL-SPECIFIC PROTEIN | | | 190) | AB023654, | 191) | U02298, X70675, | Q9UBL2 | Q5XZF2 |
| | p228\|\|SMALL INDUCIBLE | | | | AC015849, | | AF065944, | (SEQ ID NOS | (SEQ ID |
| | CYTOKINE A5\|\|chemokine | | | | AF088219, | | AF065945, | 192-194) | NOS 195-197) |
| | (C—C motif) ligand | | | | DQ017060, | | AF065946, | | |
| | 5\|\|CHEMOKINE, CC MOTIF, | | | | AF043341, | | AF065947, | | |
| | LIGAND 5\|\|REGULATED | | | | AF266753, | | AF128187, | | |
| | UPON ACTIVATION, | | | | BC008600, | | AK003101, | | |
| | NORMALLY T- | | | | BG272739, M21121, | | AK158074, | | |
| | EXPRESSED, AND | | | | BM917378 | | AY722103, | | |
| | PRESUMABLY | | | | | | BC033508, | | |
| | SECRETED\|\| | | | | | | CT010315, | | |
| | | | | | | | M77747, S37648, | | |
| | | | | | | | AI020884 | | |
| IL6 | \|\|IL6\|\|IFNB2\|\|HSF\|\|BSF2\|\| | Interleukin 6 | 3569 | NM_000600 | AC073072, | NM_031168 | AC112933, | NP_000591, | NP_112445, |
| | INTERFERON, BETA- | (interferon, | | (SEQ ID NO: | AF372214, | (SEQ ID NO: | M20572, M24221, | P05231, | P08505 |
| | 2\|\|HYBRIDOMA GROWTH | beta 2) | | 198) | CH236948, X04402, | 199) | M36996, X51457, | Q75MH2, | (SEQ ID |
| | FACTOR\|\|HEPATOCYTE | | | | Y00081, BC015511, | | AK089780, | Q8N6X1 | NOS 204-205) |
| | STIMULATORY | | | | BT019748, | | AK150440, | (SEQ ID NOS | |
| | FACTOR\|\|B-CELL | | | | BT019749, | | AK152189, | 200-203) | |
| | DIFFERENTIATION | | | | CR450296, | | J03783, X06203, | | |
| | FACTOR\|\|B-CELL | | | | CR590965, | | X54542 | | |
| | STIMULATORY FACTOR | | | | CR626263, M14584, | | | | |
| | 2\|\|Interleukin 6 (interferon, | | | | M18403, M29150, | | | | |
| | beta 2)\|\|HGF SERUM IL6 | | | | M54894, S56892, | | | | |
| | LEVEL IN INCREASED | | | | X04403, X04430, | | | | |
| | BMI, MODIFIER OF\|\| | | | | X04602, A09363 | | | | |
| IL8 | \|\|SCYB8\|\|GCP1\|\|IL8\|\|CXCL8\|\| | Interleukin 8 | 3576 | NM_000584 | AC112518, | N/A | | NP_000575, | N/A |
| | NAP1\|\|Interleukin | | | (SEQ ID NO: | AF385628, D14283, | | | P10145 | |
| | 8\|\|NEUTROPHIL- | | | 206) | M23344, | | | (SEQ ID NOS | |
| | ACTIVATING PEPTIDE | | | | M28130AJ227913, | | | 207-208) | |
| | 1\|\|MONOCYTE-DERIVED | | | | AK131067, | | | | |
| | NEUTROPHIL | | | | BC013615, | | | | |
| | CHEMOTACTIC | | | | BT007067, | | | | |
| | FACTOR\|\|GRANULOCYTE | | | | CR542151, | | | | |
| | CHEMOTACTIC PROTEIN | | | | CR594973, | | | | |
| | 1\|\|CXC CHEMOKINE | | | | CR600500, | | | | |
| | LIGAND 8\|\|SMALL | | | | CR601533, | | | | |
| | INDUCIBLE CYTOKINE | | | | CR601902, | | | | |
| | SUBFAMILY B, MEMBER | | | | CR603686, | | | | |
| | 8\|\| | | | | CR619554, | | | | |
| | | | | | CR623683, | | | | |
| | | | | | CR623827, M17017, | | | | |
| | | | | | M26383, Y00787, | | | | |
| | | | | | Z11686 | | | | |
| ICAM1 | \|\|ICAM1\|\|ANTIGEN | Intercellular | 3383 | NM_000201 | AC011511, | NM_010493 | AC159314, | NP_000192, | NM_010493, |
| | IDENTIFIED BY | adhesion | | (SEQ ID NO: | AY225514, M65001, | (SEQ ID NO: | M90546, M90547, | O00177, | P13597; |
| | MONOCLONAL | molecule 1 | | 209) | U86814, X57151, | 210) | M90548, M90549, | P05362, | Q61828 |
| | ANTIBODY | (CD54), human | | | X59286, AF340038, | | M90550, M90551, | Q14601, | (SEQ ID |
| | BB2\|\|SURFACE ANTIGEN | rhinovirus | | | AF340039, | | AK149748, | Q15463, | NOS 219-221) |
| | OF ACTIVATED B CELLS, | receptor | | | AK130659, | | AK149781, | Q5NKV7, | |
| | BB2\|\|intercellular adhesion | | | | BC015969, | | AK149945, | Q5NKV8, | |
| | molecule 1 (CD54), human | | | | BT006854, | | AK150003, | Q99930 | |
| | rhinovirus receptor\|\| | | | | CR617464, J03132, | | AK150049, | (SEQ ID NOS | |
| | | | | | M24283, M55038, | | AK150057, | 211-218) | |
| | | | | | M55091, S82847, | | AK150141, | | |
| | | | | | X06990 | | AK150327, | | |
| | | | | | | | AK151227, | | |
| | | | | | | | AK151681, | | |
| | | | | | | | AK152155, | | |
| | | | | | | | AK152527, | | |
| | | | | | | | AK152530, | | |
| | | | | | | | AK152556, | | |
| | | | | | | | AK156417, | | |
| | | | | | | | AK168275, | | |
| | | | | | | | AK171502, | | |
| | | | | | | | AK171520, | | |
| | | | | | | | AK172321, | | |
| | | | | | | | BC008626, | | |
| | | | | | | | CT010246, | | |
| | | | | | | | CT010302, | | |
| | | | | | | | M31585, X16624, | | |
| | | | | | | | X52264, X54331 | | |
| TIMP1 | \|\|TIMP1\|\|HCI\|\|EPA\|\|COLLA | TIMP | 7076 | NM_003254 | AY932824, D11139, | NM_011593 | AL671885, | NP_003245; | NP_035723, |
| | GENASE INHIBITOR, | metallopeptidase | | (SEQ ID NO: | L47361, Z84466, | (SEQ ID NO: | M21162, M28308, | Q58P21, | P12032, |
| | HUMAN\|\|TIMP | inhibitor 1 | | 222) | AK074854, | 223) | M28309, M28310, | Q5H9A7, | Q60734 |
| | metallopeptidase inhibitor | | | | BC000866, | | M28311, M28312, | Q6FGX5, | (SEQ ID |
| | 1\|\|tissue inhibitor of | | | | BC007097, | | X69413 | Q96QM2, | NOS 232-234) |
| | metalloproteinase 1 | | | | BQ181804, | | AY622853, | P01033; | |
| | (erythroid potentiating | | | | BU857950, | | BC008107, | Q14252; | |
| | activity, collagenase | | | | CR407638, | | BC034260, | Q9UCU1 | |
| | inhibitor)\|\| | | | | CR541982, | | BC051260, | (SEQ ID NOS | |
| | | | | | CR590572, | | M17243, V00755, | 224-231) | |
| | | | | | CR593351, | | X04684 | | |
| | | | | | CR602090, M12670, | | | | |
| | | | | | M59906, S68252, | | | | |
| | | | | | X02598, X03124, | | | | |
| | | | | | A10416 | | | | |
| CCL19 | \|\|CCL19\|\|ELC\|\|MIP3B\|\|SCYA19\|\| | Chemokine (C—C | 6363 | NM_006274 | AJ223410, | NM_011888 | AF307988, | NP_006265, | NP_036018, |
| | EBI1-LIGAND | motif) ligand | | (SEQ ID NO: | AL162231, | (SEQ ID NO: | AF308159, | Q6IBD6, | 070460, |
| | CHEMOKINE\|\|EXODUS | 19 | | 235) | AB000887, | 236) | AL772334, | Q99731 | Q548P0 |
| | 3\|\|MACROPHAGE | | | | BC027968, | | AF059208, | (SEQ ID NOS | (SEQ ID |
| | INFLAMMATORY | | | | CR456868, | | AK144337, | 237-239) | NOS 240-242) |
| | PROTEIN 3- | | | | CR623730, U77180, | | AK156269, | | |
| | BETA\|\|CHEMOKINE, CC | | | | U88321, BM720436 | | BC025130, | | |
| | MOTIF, LIGAND | | | | | | BC051472, | | |
| | 19\|\|chemokine (C—C motif) | | | | | | BE864988 | | |
| | ligand 19\|\|SMALL | | | | | | | | |
| | INDUCIBLE CYTOKINE | | | | | | | | |
| | SUBFAMILY A, MEMBER | | | | | | | | |
| | 19\|\| | | | | | | | | |
| CCL21 | \|\|SCYA21\|\|CCL21\|\|SLC\|\|EXODUS | Chemokine (C—C | 6366 | NM_002989 | AF030572, | NM_023052 | | NP_002980, | NP_075539 |
| | 2\|\|SECONDARY | motif) ligand | | (SEQ ID NO: | AJ005654, | (SEQ ID NO: | | O00585, | (SEQ ID |
| | LYMPHOID TISSUE | 21 | | 243) | AL162231, | 244) | | Q5VZ73, | NO: 249) |
| | CHEMOKINE\|\|CHEMOKINE, | | | | AB002409, | | | Q6ICR7 | |
| | CC MOTIF, LIGAND | | | | AF001979, | | | (SEQ ID NOS | |
| | 21\|\|chemokine (C—C motif) | | | | AY358887, | | | 245-248) | |
| | ligand 21\|\|SMALL | | | | BC027918, | | | | |
| | INDUCIBLE CYTOKINE | | | | BI833188, | | | | |
| | SUBFAMILY A, MEMBER | | | | CR450326, | | | | |
| | 21\|\| | | | | CR615435, U88320, | | | | |
| | | | | | BQ712706 | | | | |
| CSF3 | \|\|GCSF\|\|pluripoietin\|\|CSF3\|\|filgrastim\|\| | Colony | 1440 | NM_000759, | AC090844, | NM_009971 | AL590963, | NP_757374, | NP_034101, |
| | lenograstim\|\|MGC45931\|\| | stimulating | | NM_172219, | AF388025, M13008, | (SEQ ID NO: | X05402, | NP000750, | P09920 |
| | G- | factor 3 | | NM_172220 | X03656, BC033245, | 253) | AK145177, | NP75373, | (SEQ ID |
| | CSF\|\|GRANULOCYTE | (granulocyte) | | (SEQ ID NOS | CR541891, M17706, | | M13926 | P09919, | NOS 260-261) |
| | COLONY-STIMULATING | | | 250-252) | X03438, X03655 | | | Q6FH65, | |
| | FACTOR\|\|COLONY- | | | | | | | Q8N4W3 | |
| | STIMULATING FACTOR | | | | | | | (SEQ ID NOS | |
| | 3\|\|granulocyte colony | | | | | | | 254-259) | |
| | stimulating factor\|\|Colony | | | | | | | | |
| | stimulating factor 3 | | | | | | | | |
| | (granulocyte)\|\|colony | | | | | | | | |
| | stimulating factor 3 isoform | | | | | | | | |
| | c\|\|colony stimulating factor 3 | | | | | | | | |
| | isoform a precursor\|\|colony | | | | | | | | |
| | stimulating factor 3 isoform | | | | | | | | |
| | b precursor\|\| | | | | | | | | |
| TNFSF11 | \|\|ODF\|\|OPGL\|\|RANKL\|\|TRANCE\|\| | Tumor necrosis | 8600 | NM_003701, | AL139382, | NM_011613 | AB022039, | NP_143026, | NP_035743, |
| | TNFSF11\|\|OSTEOPROTEGERIN | factor (ligand) | | NM_033012 | AB037599, | (SEQ ID NO: | AC12669, | NP_003692, | O35235 |
| | LIGAND\|\|OSTEOCLAST | superfamily, | | (SEQ ID NOS | AB061227, | 264) | AB008426, | O14788, | (SEQ ID |
| | DIFFERENTIATION | member 11 | | 262-263) | AB064268, | | AB032771, | Q54A98, | NOS 270-271) |
| | FACTOR\|\|TNF-RELATED | | | | AB064269, | | AB032772, | Q5T9Y4 | |
| | ACTIVATION-INDUCED | | | | AB064270, | | AB036798, | (SEQ ID NOS | |
| | CYTOKINE\|\|RECEPTOR | | | | AF013171, | | AF013170, | 265-269) | |
| | ACTIVATOR OF NF- | | | | AF019047, | | AF019048, | | |
| | KAPPA-B LIGAND\|\|Tumor | | | | AF053712, | | AF053713, | | |
| | necrosis factor (ligand) | | | | BC074823, | | AK041129, | | |
| | superfamily, member | | | | BC074890, | | AK159498, | | |
| | 11\|\|TUMOR NECROSIS | | | | | | AK159997 | | |
| | FACTOR LIGAND | | | | | | | | |
| | SUPERFAMILY, MEMBER | | | | | | | | |
| | 11\|\| | | | | | | | | |
| IL2 | \|\|IL2\|\|TCGF\|\|Interleukin 2\|\|T- | Interleukin 2 | 3558 | NM_000586 | AC022489, | NM_008366 | AF195954, | NP_000577, | NP_032392, |
| | CELL GROWTH FACTOR\|\| | | | (SEQ ID NO: | AF031845, | (SEQ ID NO: | AF195955, | P60568, | P04351 |
| | | | | 273) | AF359939, J00264, | 274) | AF195956, | Q13169, | (SEQ ID |
| | | | | | K02056, M13879, | | AF399982, | Q16334, | NOS 286-287) |
| | | | | | M22005, M33199, | | AL645966, | Q6NZ91, | |
| | | | | | X00695, X61155, | | AL662823, | Q6NZ93, | |
| | | | | | AF228636, | | L07574, L07576, | Q6QWN0, | |
| | | | | | AF532913, | | M16760, M16761, | Q71V48, | |
| | | | | | AY283686, | | M16762, X01663, | Q7Z7M3, | |
| | | | | | AY523040, | | X01664, X01665, | Q8NFA4, | |
| | | | | | BC066254, | | X52618, | Q9C001 | |
| | | | | | BC066255, | | AF065914, | (SEQ ID NOS | |
| | | | | | BC066256, | | AF065915, | 275-285) | |
| | | | | | BC066257, | | AF065916, | | |
| | | | | | BC070338, | | AF352786, | | |
| | | | | | DQ231169, S77834, | | AF538059, | | |
| | | | | | S77835, S82692, | | AF542383, | | |
| | | | | | U25676, V00564, | | AF542384, | | |
| | | | | | X01586, A14844 | | AF542385, | | |
| | | | | | | | AY147902, | | |
| | | | | | | | K02292, U41494, | | |
| | | | | | | | U41504, U41505, | | |
| | | | | | | | U41506, X01772, | | |
| | | | | | | | X66058, X73040 | | |
| IL4 | \|\|IL4\|\|BSF1\|\|Interleukin 4\|\|B- | Interleukin 4 | 3565 | NM_000589, | AC004039, | NM_021283 | AC005742, | NP_758858, | NP_067258, |
| | CELL STIMULATORY | | | NM_172348 | AF395008, | (SEQ ID NO: | AL596095, | P05112, | P07750, |
| | FACTOR 1\|\| | | | (SEQ ID NOS | AF465829, M23442, | 290) | AL645741, | Q5FC01, | Q5SV00 |
| | | | | 288-289) | X06750, AB102862, | | U07869, X05064, | Q6NWP0, | (SEQ ID |
| | | | | | AF043336, | | X05252, X05253, | Q6NZ77, | NOS 297-299) |
| | | | | | BC066277, | | AB174765, | Q9UPB9 | |
| | | | | | BC066278, | | AF352783, | (SEQ ID NOS | |
| | | | | | BC067514, | | BC027514, | 291-296) | |
| | | | | | BC067515, | | M13238, M25892, | | |
| | | | | | BC070123, M13982, | | X03532 | | |
| | | | | | X81851 | | | | |
| IL13 | \|\|IL13\|\|Interleukin 13\|\| | Interleukin 13 | 3596 | NM_002188 | AC004039, | NM_008355 | AC005742, | NP_002179, | NP_032381, |
| | | | | (SEQ ID NO: | AF172149, | (SEQ ID NO: | AL645741, | P35225, | P20109, |
| | | | | 300) | AF172150, | 301) | L13028, M23504 | Q4VB50, | Q5SUZ9 |
| | | | | | AF193838, | | | Q4VB51, | (SEQ ID |
| | | | | | AF193839, | | | Q4VB52, | NOS 308-310) |
| | | | | | AF193840, | | | Q4VB53 | |
| | | | | | AF377331, | | | (SEQ ID NOS | |
| | | | | | AF416600, | | | 302-307) | |
| | | | | | AY008331, | | | | |
| | | | | | AY008332, L13029, | | | | |
| | | | | | L42079, L42080, | | | | |
| | | | | | U10307, U31120, | | | | |
| | | | | | AF043334, | | | | |
| | | | | | BC096138, | | | | |
| | | | | | BC096139, | | | | |
| | | | | | BC096140, | | | | |
| | | | | | BC096141, L06801, | | | | |
| | | | | | X69079 | | | | |
| IL1b | \|\|IL1B\|\|IL1- | Interleukin 1, | 3553 | NM_000576 | AC079753, | NM_008361 | AL808143, | NP_000567, | NP_032387, |
| | BETA\|\|INTERLEUKIN 1- | beta | | (SEQ ID NO: | AY137079, | (SEQ ID NO: | AY902319, | O43645, | P10749 |
| | BETA\|\|Interleukin 1, beta\|\| | | | 311) | BN000002, M15840, | 312) | U03987, X04964, | P01584, | (SEQ ID |
| | | | | | X04500, X52430, | | AK156396, | Q53X59, | NOS 318-319) |
| | | | | | X52431, AF043335, | | AK157245, | Q53XX2 | |
| | | | | | BC008678, | | AK168047, | (SEQ ID NOS | |
| | | | | | BT007213, | | BC011437, | 313-317) | |
| | | | | | CR407679, K02770, | | M15131 | | |
| | | | | | M15330, M54933, | | | | |
| | | | | | X02532, X56087 | | | | |
| CCL12 | | mouse protein | | | | NM_011331 | AL645596, | | NP_035461, |
| | | only | | | | (SEQ ID NO: | AF065934, | | Q5SVB4, |
| | | | | | | 320) | AF065935, | | Q62401, |
| | | | | | | | AF065936, | | Q9QYD6 |
| | | | | | | | AF065937, | | (SEQ ID |
| | | | | | | | AF065938, | | NOS 321-324) |
| | | | | | | | AK012356, | | |
| | | | | | | | BC027520, | | |
| | | | | | | | U50712, U66670 | | |
| CCL9 | | mouse protein | | | | NM_011338 | AB051897, | | NP_035468; |
| | | only | | | | (SEQ ID NO: | AL596122, | | P51670; |
| | | | | | | 325) | AY902335, | | Q5QNW2 |
| | | | | | | | AF128195, | | (SEQ ID |
| | | | | | | | AF128196, | | NOS 326-328) |
| | | | | | | | AF128197, | | |
| | | | | | | | AF128198, | | |
| | | | | | | | AF128199, | | |
| | | | | | | | AF128200, | | |
| | | | | | | | AF128201, | | |
| | | | | | | | AF128202, | | |
| | | | | | | | AF128203, | | |
| | | | | | | | AF128204, | | |
| | | | | | | | AI323857, | | |
| | | | | | | | AK151131, | | |
| | | | | | | | AK151340, | | |
| | | | | | | | AK151649, | | |
| | | | | | | | AK151953, | | |
| | | | | | | | AK154511, | | |
| | | | | | | | AK154657, | | |
| | | | | | | | AK155032, | | |
| | | | | | | | AK155036, | | |
| | | | | | | | U15209, U19482, | | |
| | | | | | | | U49513 | | |
| CXCL1 | \|\|CXCL1\|\|NAP-3\|\|MGSA- | Chemokine | 2919 | NM_001511 | AC092438, U03018, | NM_008176 | AC157938 | NP_001502, | NP_032202, |
| | a\|\|SCYB1\|\|GROa\|\|MGSA | (C—X—C motif) | | (SEQ ID NO: | X54489, BC011976, | (SEQ ID NO: | (110717..112522), | P09341, | P12850, |
| | alpha\|\|GRO PROTEIN, | ligand 1 | | 329) | BT006880, J03561, | 330) | S79767, U20527, | Q6LD34 | Q5U5W9 |
| | ALPHA\|\|MELANOMA | (melanoma | | | X12510, BF032655 | | U20634, | (SEQ ID NOS | (SEQ ID |
| | GROWTH STIMULATORY | growth | | | | | AK140312, | 331-333) | NOS 334-336) |
| | ACTIVITY, | stimulating | | | | | BC037997, | | |
| | ALPHA\|\|melanoma growth | activity, alpha) | | | | | BG067198, | | |
| | stimulatory activity | | | | | | BG080268, | | |
| | alpha\|\|KC CHEMOKINE, | | | | | | J04596, | | |
| | MOUSE, HOMOLOG | | | | | | BQ031102 | | |
| | OF\|\|CHEMOKINE, CXC | | | | | | | | |
| | MOTIF, LIGAND 1\|\|GRO1 | | | | | | | | |
| | oncogene (melanoma | | | | | | | | |
| | growth-stimulating | | | | | | | | |
| | activity)\|\|GRO1 oncogene | | | | | | | | |
| | (melanoma growth | | | | | | | | |
| | stimulating activity, | | | | | | | | |
| | alpha)\|\|SMALL | | | | | | | | |
| | INDUCIBLE CYTOKINE | | | | | | | | |
| | SUBFAMILY B, MEMBER | | | | | | | | |
| | 1\|\|chemokine (C—X—C motif) | | | | | | | | |
| | ligand 1 (melanoma growth | | | | | | | | |
| | stimulating activity, alpha)\|\| | | | | | | | | |
| CXCL2 | \|\|MIP2A\|\|GROb\|\|MGSA- | Chemokine | 2920 | NM_002089 | AC093677 | NM_009140 | AC157938, | NP_002080, | NP_033166, |
| | b\|\|MIP2- | (C—X—C motif) | | (SEQ ID NO: | (22698..24854, | (SEQ ID NO: | S61346, | P19875, | P10889 |
| | ALPHA\|\|SCYB2\|\|CXCL2\|\|MIP- | ligand 2 | | 337) | complement), | 338) | AK137628, | Q6FGD6, | (SEQ ID |
| | 2a\|\|CINC-2a\|\|GRO2 | | | | U03019, AF043340, | | AK150450, | Q6LD33 | NOS 343-344) |
| | oncogene\|\|MGSA beta\|\|GRO | | | | BC005276, | | AK155458, | (SEQ ID NOS | |
| | PROTEIN, | | | | BC015753, | | AK155874, | 339-342) | |
| | BETA\|\|MACROPHAGE | | | | BC053653, | | AK155916, | | |
| | INFLAMMATORY | | | | CR542171, | | AK157079, | | |
| | PROTEIN 2\|\|melanoma | | | | CR617096, M36820, | | X53798 | | |
| | growth stimulatory activity | | | | M57731, X53799 | | | | |
| | beta\|\|CHEMOKINE, CXC | | | | | | | | |
| | MOTIF, LIGAND | | | | | | | | |
| | 2\|\|chemokine (C—X—C motif) | | | | | | | | |
| | ligand 2\|\|SMALL | | | | | | | | |
| | INDUCIBLE CYTOKINE | | | | | | | | |
| | SUBFAMILY B, MEMBER | | | | | | | | |
| | 2\|\| | | | | | | | | |
| IL12B | \|\|NKSF2\|\|CLMF2\|\|IL12B\|\|IL12, | Interleukin 12B | 3593 | NM_002187 | AC011418, | NM_008352 | AL607030, | NP_002178, | NP_032378, |
| | SUBUNIT p40\|\|IL23, | (natural killer | | (SEQ ID NO: | AF512686, | (SEQ ID NO: | AL669944, | P29460, | P43432 |
| | SUBUNIT p40\|\|NATURAL | cell stimulatory | | 345) | AY008847, | 346) | D63333, S82421, | Q8NOX8 | (SEQ ID |
| | KILLER CELL | factor 2, | | | AY064126, U89323, | | S82422, S82424, | (SEQ ID NOS | NOS 350-351) |
| | STIMULATORY FACTOR, | cytotoxic | | | AF180563, | | S82425, S82426, | 347-349) | |
| | 40-KD | lymphocyte | | | AY046592, | | AF128214, | | |
| | SUBUNIT\|\|interleukin 12B | maturation | | | AY046593, | | AF128215, | | |
| | (natural killer cell | factor 2, p40) | | | BC067498, | | AK155593, | | |
| | stimulatory factor 2, | | | | BC067499, | | AK162981, | | |
| | cytotoxic lymphocyte | | | | BC067500, | | BC103608, | | |
| | maturation factor 2, p40)\|\| | | | | BC067501, | | BC103609, | | |
| | | | | | BC067502, | | BC103610, | | |
| | | | | | BC074723, M65272, | | BC103614, | | |
| | | | | | M65290 | | M86671 | | |
| LEP | \|\|LEP\|\|Leptin (obesity homolog, | Leptin (obesity | 3952 | NM_000230 | AC018635, AC018662, | NM_008493 | AC072048, U22421, | NP_000221, | NP_032519, |
| | mouse)\|\|LEP OBESE, MOUSE, | homolog, mouse) | | (SEQ ID | AY996373, CH236947, | (SEQ ID NO: 353) | U52147, AK030984, | P41159, Q4TVR7, | P41160, |
| | HOMOLOG OF\|\| | | | NO: 352) | D63519, D63710, | | AK142589, | Q6NT58 | Q544U0 |
| | | | | | DQ054472, U43415, | | BC038162, U18812 | (SEQ ID NOS | (SEQ ID |
| | | | | | AF008123, BC060830, | | | 354-357) | NOS 358-360) |
| | | | | | BC069323, BC069452, | | | | |
| | | | | | BC069527, D49487, | | | | |
| | | | | | U18915, U43653 | | | | |

In addition to the specific biomarker sequences identified in this application by name, accession number, or sequence, the invention also contemplates contemplates use of biomarker variants that are at least 90% or at least 95% or at least 97% identical to the exemplified sequences and that are now known or later discover and that have utility for the methods of the invention. These variants may represent polymorphisms, splice variants, mutations, and the like. Various techniques and reagents find use in the diagnostic methods of the present invention. In one embodiment of the invention, blood samples, or samples derived from blood, e.g. plasma, circulating, etc. are assayed for the presence of polypeptides. Typically a blood sample is drawn, and a derivative product, such as plasma or serum, is tested. Such polypeptides may be detected through specific binding members. The use of antibodies for this purpose is of particular interest. Various formats find use for such assays, including antibody arrays; ELISA and RIA formats; binding of labeled antibodies in suspension/solution and detection by flow cytometry, mass spectroscopy, and the like. Detection may utilize one or a panel of antibodies, preferably a panel of antibodies in an array format. Expression signatures typically utilize a detection method coupled with analysis of the results to determine if there is a statistically significant match with a disease signature.

In another embodiment, in vivo imaging is utilized to detect the presence of atherosclerosis associated proteins in heart tissue. Such methods may utilize, for example, labeled antibodies or ligands specific for such proteins. In these embodiments, a detectably-labeled moiety, e.g., an antibody, ligand, etc., which is specific for the polypeptide is administered to an individual (e.g., by injection), and labeled cells are located using standard imaging techniques, including, but not limited to, magnetic resonance imaging, computed tomography scanning, and the like. Detection may utilize one or a cocktail of imaging reagents.

In another embodiment, an mRNA sample from vessel tissue, preferably from one or more vessels affected by atherosclerosis, is analyzed for the genetic signature indicating atherosclerosis.

The provided patterns of circulating protein expression characterize the inflammatory signature in atherosclerosis, and further links specific immune related pathways to diabetes and medication therapy. While current data suggests a significant role for inflammation in atherosclerosis, there remains little direct data linking immune pathways in the vessel wall to critical aspects of the disease, including the mechanisms by which risk factors impact the primary inflammatory process, and how medications that modify risk factors such as hypertension and hyperlipidemia may specifically impact inflammation. The present invention identifies expression profiles of biomarkers of inflammation that can be used for diagnosis and classification of atherosclerotic cardiovascular disease.

In methods of diagnosing a patient for atherosclerosis and related conditions, the expression pattern in blood, serum, etc. of the markers provided herein is obtained, and compared to control values to determine a diagnosis. The analysis of the invention may further include input from clinical variables. For example, a blood derived patient sample, e.g. blood, plasma, serum, etc. may be applied to a specific binding agent or panel of specific binding agents, to determine the presence of the markers of interest. The analysis will generally include at least one of the markers described herein, e.g., M-CSF, eotaxin, IP-10, MCP-1, MCP-2, MCP-3, MCP-4, IL-3, IL-5, IL-7, IL-8, MIP1a, TNFa, Ang-2, IGF-1 and RANTES, usually at least two of the markers, more usually at least three of the markers, and may include 4, 5, 6, 7 or up to all of the markers. A preferred set of markers comprises at least three of the following: MCP-1, MCP-2, MCP-3, MCP-4, eotaxin, IP-10, M-CSF, IL-3, TNFa, Ang-2, IL-5, IL-7 and TGF-1, and may include, 4, 5, 6, 7, 8, 9, 10, 11, 12, or all of them.

The analysis may further comprise the inclusion of expression information from additional proteins, which may be present in serum or in tissue samples. Quantitative information will be obtained by methods suitable for the marker. Markers include, without limitation, sVCAM; sICAM-1; E-selectin; P-selection; interleukin-6, interleukin-18; creatine kinase; LDL, oxLDL, LDL particle size, Lipoprotein(a); troponin I, troponin T; LPLA2; CRP; Ccl9; Ccl2; Ccl21; Ccl19; IL-5; Tnfsf11; Vegfa; Cxcl1; leptin, HDL, Triglyceride, insulin, BNP (brain naturetic peptide), fractalkine, osteopontin, osteoprotegerin, oncostatin-M, Myeloperoxidase, ADMA, PAI-1 (plasminogen activator inhibitor), SAA (serum amyloid A), t-PA (tissue-type plasminogen activator), sCD40 ligand, fibrinogen, homocysteine, D-dimer, leukocyte count, etc. Additional variables include clinical indicia, which will typically be assessed and the resulting data combined in an algorithm with the circulating marker analysis. Such clinical markers include, without limitation: gender; age; glucose; insulin; body mass index (BMI); heart rate; waist size; systolic blood pressure; diastolic blood pressure; dyslipidemia; cigarette smoking; and the like. Other variables include metabolic measures, genetic information, and gene expression measures from peripheral blood.

The methods of the invention may be used for atherosclerosis staging, atherosclerosis prognosis, assessing extent of atherosclerosis progression, monitoring a therapeutic response, etc. One of ordinary skill having the benefit of this disclosure will readily understand how to practice the invention for these uses. For example, atherosclerosis staging may be accomplished by comparison of an individual dataset against with one or more datasets obtained from disease samples of known stage or by constructing a model that predicts stage and inputting a dataset in that model to obtain a predicted staging. Similar methods may be used to provide atherosclerosis prognosis. Progression may be monitored, by looking at changes over time in one or more predictors obtained from a predictive model such as, e.g., a model described infra. Therapeutic responses may be determined by using the methods of the invention and determining whether one or more classifications obtained from a subject with known disease trend toward or lie within a normal classification.

The quantitation of markers in a test sample is determined by the methods described above and as known in the art. The quantitative data thus obtained is then subjected to an analytic classification process. In such a process, the raw data is manipulated according to an algorithm, where the algorithm has been pre-defined by a training set of data, for example as described in the examples provided herein. An algorithm may utilize the training set of data provided herein, or may utilize the guidelines provided herein to generate an algorithm with a different set of data.

An analytic classification process may use any one of a variety of statistical analytic methods to manipulate the quantitative data and provide for classification of the sample. Examples of useful methods include linear discriminant analysis, recursive feature elimination, a prediction analysis of microarray, a logistic regression, a CART algorithm, a FlexTree algorithm, a LART algorithm, a random forest algorithm, a MART algorithm, machine learning algorithms; etc.

Using any one of these methods, an atherosclerosis dataset is used to generate a predictive model. In the generation of such a model, a dataset comprising control and diseased samples is used as a training set. A training set will contain data for each of the markers of interest. Examples of predictive models for markers of interest are provided herein, for example see Examples 6-10.

The predictive models demonstrated herein utilize the results of multiple protein level determinations, and provide an algorithm that will classify with a desired degree of accuracy an individual as belonging to a particular state, where a state may be atherosclerotic or non-atherosclerotic. Classification of interest include, without limitation, the assignment of a sample to one or more of the atherosclerotic disease states i) atherosclerotic state vs. non-atherosclerotic state, ii) MI state vs. angina state, iii) low calcium state versus high calcium state.

Classification can be made according to predictive modeling methods that set a threshold for determining the probability that a sample belongs to a given class. The probability preferably is at least 50%, or at least 60% or at least 70% or at least 80% or higher. Classifications also may be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model may be evaluated according to its ability to provide a quality metric, e.g. AUC or accuracy, of a particular value, or range of values. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold may refer to a predictive model that will classify a sample with an AUC (area under the curve) of at least about 0.7, at least about 0.75, at least about 0.8, at least about 0.85, at least about 0.9, or higher.

As is known in the art, the relative sensitivity and specificity of a predictive model can be “tuned” to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity may be at least about at least about 0.7, at least about 0.75, at least about 0.8, at least about 0.85, at least about 0.9, or higher.

The raw data may be initially analyzed by measuring the values for each marker, usually in triplicate or in multiple triplicates. The data may be manipulated, for example, raw data may be transformed using standard curves, and the average of triplicate measurements used to calculate the average and standard deviation for each patient. These values may be transformed before being used in the models, e.g. log-transformed, Box-Cox transformed (see Box and Cox (1964) J. Royal Stat. Soc., Series B, 26:211-246), etc. The data are then input into a predictive model, which will classify the sample according to the state. The resulting information may be transmitted to a patient or health professional.

To generate a predictive model for atherosclerotic states, a robust data set, comprising known control samples and samples corresponding to the atherosclerotic classification of interest is used in a training set. A sample size is selected using generally accepted criteria. As discussed above, different statistical methods can be used to obtain a highly accurate predictive model. Examples of such analysis are provided in Examples 5, 11 and 12.

In one embodiment, hierarchical clustering is performed in the derivation of a predictive model, where the Pearson correlation is employed as the clustering metric. One approach is to consider a patient atherosclerosis dataset as a “learning sample” in a problem of “supervised learning”. CART is a standard in applications to medicine (Singer (1999) Recursive Partitioning in the Health Sciences, Springer), which may be modified by transforming any qualitative features to quantitative features; sorting them by attained significance levels, evaluated by sample reuse methods for Hotelling's T² statistic; and suitable application of the lasso method. Problems in prediction are turned into problems in regression without losing sight of prediction, indeed by making suitable use of the Gini criterion for classification in evaluating the quality of regressions.

This approach has led to what is termed FlexTree (Huang (2004) PNAS 101:10529-10534). FlexTree has performed very well in simulations and when applied to SNP and other forms of data. Software automating FlexTree has been developed. Alternatively LARTree or LART may be used. Fortunately, recent efforts have led to the development of such an approach, termed LARTree (or simply LART) Turnbull (2005) Classification Trees with Subset Analysis Selection by the Lasso, Stanford University. The name reflects binary trees, as in CART and FlexTree; the lasso, as has been noted; and the implementation of the lasso through what is termed LARS by Efron et al. (2004) Annals of Statistics 32:407-451. See, also, Huang et al. (2004) Tree-structured supervised learning and the genetics of hypertension. Proc Natl Acad Sci USA. 101(29):10529-34.

Other methods of analysis that may be used include logic regression. One method of logic regression Ruczinski (2003) Journal of Computational and Graphical Statistics 12:475-512. Logic regression resembles CART in that its classifier can be displayed as a binary tree. It is different in that each node has Boolean statements about features that are more general than the simple “and” statements produced by CART.

Another approach is that of nearest shrunken centroids (Tibshirani (2002) PNAS 99:6567-72). The technology is k-means-like, but has the advantage that by shrinking cluster centers, one automatically selects features (as in the lasso) so as to focus attention on small numbers of those that are informative. The approach is available as PAM software and is widely used. Two further sets of algorithms are random forests (Breiman (2001) Machine Learning 45:5-32 and MART (Hastie (2001) The Elements of Statistical Learning, Springer). These two methods are already “committee methods.” Thus, they involve predictors that “vote” on outcome.

To provide significance ordering, the false discovery rate (FDR) may be determined. First, a set of null distributions of dissimilarity values is generated. In one embodiment, the values of observed profiles are permuted to create a sequence of distributions of correlation coefficients obtained out of chance, thereby creating an appropriate set of null distributions of correlation coefficients (see Tusher et al. (2001) PNAS 98, 5116-21, herein incorporated by reference). The set of null distribution is obtained by: permuting the values of each profile for all available profiles; calculating the pair-wise correlation coefficients for all profile; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value may be applied to the correlations between experimental profiles.

Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pairwise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation.

In an alternative analytical approach, variables chosen in the cross-sectional analysis are separately employed as predictors. Given the specific ASCVD outcome, the random lengths of time each patient will be observed, and selection of proteomic and other features, a parametric approach to analyzing survival may be better than the widely applied semi-parametric Cox model. A Weibull parametric fit of survival permits the hazard rate to be monotonically increasing, decreasing, or constant, and also has a proportional hazards representation (as does the Cox model) and an accelerated failure-time representation. All the standard tools available in obtaining approximate maximum likelihood estimators of regression coefficients and functions of them are available with this model.

In addition the Cox models may be used, especially since reductions of numbers of covariates to manageable size with the lasso will significantly simplify the analysis, allowing the possibility of an entirely nonparametric approach to survival. These statistical tools are applicable to all manner of proteomic data. A set of biomarker, clinical and genetic data that can be easily determined, and that is highly informative regarding detection of individuals with clinically significant atherosclerotic coronary vascular disease is provided. Also, algorithms provide information regarding risk of future cardiovascular events.

In the development of a predictive model, it may be desirable to select a subset of markers, i.e. at least 3, at least 4, at least 5, at least 6, up to the complete set of markers. Usually a subset of markers will be chosen that provides for the needs of the quantitative sample analysis, e.g. availability of reagents, convenience of quantitation, etc., while maintaining a highly accurate predictive model.

The selection of a number of informative markers for building classification models requires the definition of a performance metric and a user-defined threshold for producing a model with useful predictive ability based on this metric. For example, the performance metric may be the AUC, the sensitivity and/or specificity of the prediction as well as the overall accuracy of the prediction model.

As described in Examples 5, 11 and 12, various methods are used in a training model. The selection of a subset of markers may be for a forward selection or a backward selection of a marker subset. The number of markers may be selected that will optimize the performance of a model without the use of all the markers. One way to define the optimum number of terms is to choose the number of terms that produce a model with desired predictive ability (e.g. an AUC>0.75, or equivalent measures of sensitivity/specificity) that lies no more than one standard error from the maximum value obtained for this metric using any combination and number of terms used for the given algorithm.

### Reagents and Kits

Also provided are reagents and kits thereof for practicing one or more of the above-described methods. The subject reagents and kits thereof may vary greatly. Reagents of interest include reagents specifically designed for use in production of the above described expression profiles of circulating protein markers associated with atherosclerotic conditions.

One type of such reagent is an array or kit of antibodies that bind to a marker set of interest. A variety of different array formats are known in the art, with a wide variety of different probe structures, substrate compositions and attachment technologies. Representative array or kit compositions of interest include or consist of reagents for quantitation of at least two, at least three, at least four, at least five or more markers are selected from M-CSF, eotaxin, IP-10, MCP-1, MCP-2, MCP-3, MCP-4, IL-3, IL-5, IL-7, IL-8, MIP1a, TNFa, and RANTES.

In other embodiments, a representative array or kit includes or consists of reagents for quantitation of at least three protein markers selected from the group consisting of MCP-1, MCP-2, MCP-3, MCP-4, eotaxin, IP-10, M-CSF, IL-3, TNFa, Ang-2, IL-5, IL-7, and IGF-1. The at least three protein markers may comprise or consist of a marker set selected from the group consisting of MCP-1, IGF-1, TNFa; MCP-1, IGF-1, M-CSF; ANG-2, IGF-1, M-CSF; and MCP-4, IGF-1, M-CSF.

In other embodiments, a representative array or kit includes or consists of reagents for quantitation of at least four protein markers selected from the group consisting of MCP-1, MCP-2, MCP-3, MCP-4, eotaxin, IP-10, M-CSF, IL-3, TNFa, Ang-2, IL-5, IL-7, and IGF-1. The at least four protein markers comprise or consist of MCP-1, MCP-2, MCP-3, MCP-4, eotaxin, IP-10, M-CSF, IL-3, TNFa, Ang-2, IL-5, IL-7, and IGF-1; MCP-1, IGF-1, TNFa, IL-5; MCP-1, IGF-1, M-CSF, MCP-2; ANG-2, IGF-1, M-CSF, IL-5; MCP-1, IGF-1, TNFa, MCP-2; and MCP-4, IGF-1, M-CSF, IL-5.

In other embodiments, a representative array or kit includes or consists of reagents for quantitation of at least five protein markers selected from the group consisting of MCP-1, MCP-2, MCP-3, MCP-4, eotaxin, IP-10, M-CSF, IL-3, TNFa, Ang-2, IL-5, IL-7, and IGF-1. The at least five markers may comprise or consist of a marker set selected from the group consisting of MCP-1, MCP-2, MCP-3, MCP-4, eotaxin, IP-10, M-CSF, IL-3, TNFa, Ang-2, IL-5, IL-7, and IGF-1; MCP-1, IGF-1, TNFa, IL-5, M-CSF; MCP-1, IGF-1, M-CSF, MCP-2, IP-10; ANG-2, IGF-1, M-CSF, IL-5, TNFa; MCP-1, IGF-1, TNFa, MCP-2, IP-10; MCP-4, IGF-1, M-CSF, IL-5, TNFa; and MCP-4, IGF-1, M-CSF, IL-5, MCP-2.

The kits may further include a software package for statistical analysis of one or more phenotypes, and may include a reference database for calculating the probability of classification. The kit may include reagents employed in the various methods, such as devices for withdrawing and handling blood samples, second stage antibodies, ELISA reagents; tubes, spin columns, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1

### Serum Markers in an Animal Model for Atherosclerosis

Serum Biomarker Data from Mouse Protein Arrays

Given the involvement of multiple biological pathways identified through transcriptional profiling of human and mouse vascular tissue, a proof of concept study in mice was designed to examine whether a multi-analyte approach can lead to improved distinction among various stages of the atherosclerotic disease process³². The study demonstrated that quantification of multiple disease related biomarkers can provide a more sensitive and specific methodology for assessing atherosclerotic disease in mice and possibly in humans. The top serum protein classifiers identified in the study represented diverse atherosclerosis related biological processes including macrophages chemoattraction (Ccl9, Ccl2), T-cell chemokine activity (Ccl21 and Ccl19), innate immunity (IL-5), vascular calcification (Tnfsf11), angiogenesis (Vegfa), and high fat induced inflammation (Cxcl1, leptin). The signature pattern derived from simultaneous measurement of these markers added to the specificity needed for correct staging of atherosclerotic disease in mice. Further validation of this approach was obtained in prospective cohort studies in humans as described in Examples 3 and 4, below.

To identify patterns of serum protein expression that can be correlated to both disease progression and gene expression in the vascular wall, we have taken advantage of a longitudinal experimental design and mouse genetic model and diet combinations that produce varying degrees of atherosclerosis. Here, we have utilized a protein microarray to identify a set of inflammatory biomarkers that are differentially expressed in the sera of mice at levels that correlate with various severity levels of disease. The vascular wall gene expression for a subset of these markers was also evaluated by quantitative real-time reverse transcriptase polymerase chain reaction (RTPCR). Using classification algorithms to identify a set of the most sensitive discriminators, we were able to show that unique signature patterns of vascular-derived inflammatory biomarkers can accurately predict different severities of atherosclerotic disease in mice.

Methods

Experimental design, serum collection, and RNA preparation. All experiments were approved by the Stanford Committee on Animal Research. The general experimental design has been described previously (45). Three-week-old female apoE knockout (C57BL/6J-Apoetm1Unc), C57B1/6J, and C3H/HeJ mice were purchased from Jackson Laboratory (Bar Harbor, Me.). At 4 wk of age, the mice were either continued on normal chow or were fed a high-fat diet that included 21% anhydrous milkfat and 0.15% cholesterol (Dyets no. 101511; Dyets, Bethlehem, Pa.) for a maximum period of 40 wk. Serum was collected by retroorbital approach for five to nine individual mice at every time point for apoE-deficient mice on the high-fat diet from the same cohort of mice as described previously. To control for diet and genetic differences, serum was also collected at baseline and at 40 wk from apoE knockout mice (C57BL/6J-Apoetm1Unc) on normal chow and from wild-type C57B1/6J and C3H/HeJ mice on normal chow and high-fat diets. Aortas from 15 mice (3 pools of 5) were harvested for RNA isolation, as described previously (45), at each of the time points for each of the conditions (strain-diet combination) to parallel serum collection schedule. Total RNA was isolated as described previously using a modified two-step purification protocol (45, 47). Quantification of aortic atherosclerotic plaque (determined as percent lesion area in entire aorta) previously has been performed on this cohort of mice and described in a prior publication (45). Serum and aortas from a separate independent cohort of 16-wk old apoE-deficient mice on high-fat diet for 2 wk (4 pools of 3-4 animals) were also used for classification purposes. The rationale for pooling RNA and serum samples for microarray hybridizations has been discussed previously (45-47, 49). All sample processing and protein hybridization were performed at the same time to negate any potential technical variability.

Protein biochip hybridization and data processing. Serum samples were hybridized to Zyomyx Murine Cytokine BioChips (Zyomyx, Hayward, Calif.) following the manufacturer's instructions, using the Zyomyx 1200 Assay station (Zyomyx). Nine-point calibration curves were generated for each analyte for accurate determination of protein levels in test sera (please see Supplement S4 for individual calibration curves; available at the Physiological Genomics web site). 1 Protein biochips were scanned using a Zyomyx 100 fluorescence scanner, and microarray gridding was performed using GenPix Pro and Zyomyx ZDR version 4001 software. Intrachip (ratio of standard deviation of all negative control features over the average intensity for those features) and interchip variability (ratio of average standard deviation over average of median intensities) were determined as measures of quality control. Protein arrays present control variability ranging from 3 to ˜15% and sensitivity from 1 to 1,000 pg/ml depending on the analyte (see Supplemental Calibration Curves for each analyte available at http://physiolgenomics.physiology.org/cgi/content/full/00240.2005/DC1) (11). Values that were not in the linear portion of the calibration curves were marked as missing values. Numerical raw data were then migrated into an Oracle relational database (CoBi) that has been designed specifically for microarray data analysis (GeneData). Heat maps were generated using HeatMap Builder software (7). Detailed Supplemental Methods are available at http://physiolgenomics.physiology.org/cgi/content/full/00240.2005/DC1.

Protein selection algorithms and disease classification. Protein selection and classification algorithms have been described previously (45). Briefly, for supervised analyses, we used Expressionist software version 5.0 (GeneData), which employs a number of classification algorithms to rank genes based on their utility for class discrimination between time points of 0, 10, 24, and 40 wk in apoE mice on high-fat diet. These algorithms included analysis of variance (ANOVA), support vector machine (SVM) (4), and recursive feature elimination (RFE) (16), which is a recursive version of the SVM weight where genes are ranked repeatedly and a fixed fraction of worst scorers are removed each time (35). We also used the previously described prediction analysis of microarray (PAM) as an additional classification algorithm (48). Each method was then used to determine the optimal number of ranked genes to classify the experiments into their correct groups at minimal error rate. The optimal error rate or misclassification was calculated by cross-validation with 25% of the experiments as the test group and the rest as the training group. This was reiterated 1,000 times for ANOVA, SVM, and RFE algorithms. In our analyses, we used a linear kernel for SVM and RFE; a nonlinear Gaussian kernel yielded similar results. This minimal subset of classifier genes was then used for cross-validation as well as classification of another independent data set. Detailed methods are provided in http://physiolgenomics.physiology.org/cgi/content/full/00240.2005/DC1.

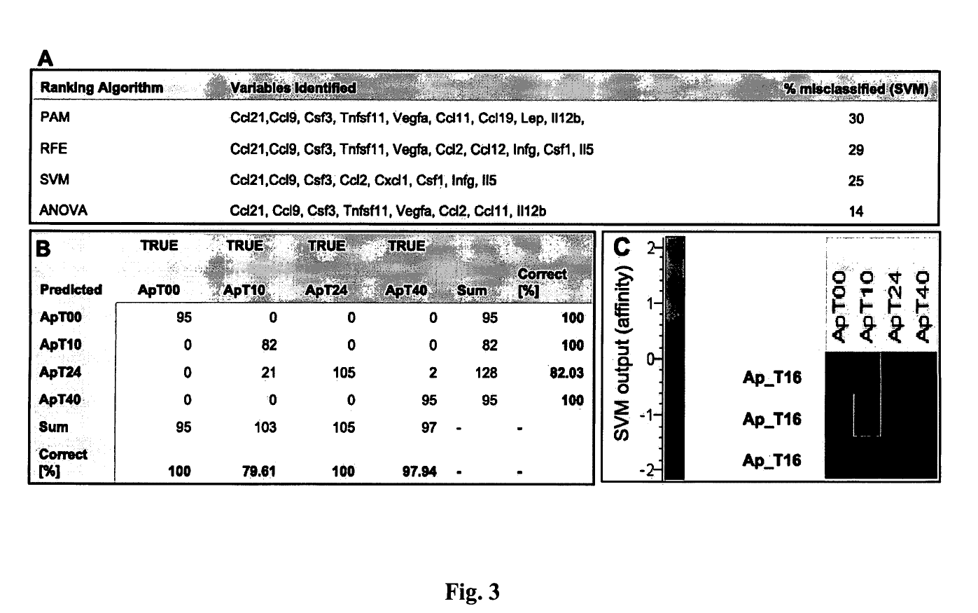

Cross-validation and analysis of independent data sets. To determine the accuracy of classification based on the small subset of proteins identified earlier, we utilized the SVM algorithm (linear kernel) to generate a confusion matrix using cross-validation with repeated splits into 75% training and 25% test sets. Results are represented in tabular fashion. We also utilized the SVM algorithm for classification of independent groups of experiments as described previously (45, 50). In this analysis, we used the four time points in apoE-deficient mice as the training set and the independent set of experiments as the test set. SVM output for each experiment based on one-vs.-all comparisons was represented graphically in a heat map format (see FIG. 3), which is the normalized margin value for each of the four SVM classifiers mentioned above. The SVM output allows us to view how a new experiment is classified according to the four SVM hyperplanes. Detailed methods are available at http://physiolgenomics.physiology.org/cgi/content/full/00240.2005/DC1.

Quantitative real-time RT-PCR. Primers and probes for 10 genes of interest were obtained from Applied Biosystems Assays-on-Demand for Taqman analysis (Table 2).

TABLE 2

| Zymomyx Mu_chip | Name | Mm_Symbol | Hs_Symbol | UGCIuster | Mm_LLID | UGCIuster | Hs_LLID | Mm_ABI-Taqman |
|---|---|---|---|---|---|---|---|---|
| Mu_Eotaxin | Eotaxin | Ccl11 | CCL11 | Mm.4686 | 20292 | Hs.54460 | 6356 | Mm00441238_m1 |
| Mu_MIP-3b | MIP-3b | Ccl19 | CCL19 | | | Hs.50002 | 6363 | Mm00839967_g1 |
| Mu_MCP-1 | MCP-1 | Ccl2 | CCL2 | Mm.290320 | 20296 | Hs.303649 | 6347 | Mm00441242_m1 |
| Mu_TCA4/6Ckine | TCA4/6Ckine | Ccl21 | CCL21 | | | Hs.57907 | 6366 | Custom Design |
| Mu_MIP-1g | MIP-1g | Ccl9 | CCL9 | Mm.2271 | 20308 | | | Mm00441260_m1 |
| Mu_GCSF | GCSF | Csf3 | CSF3 | Mm.1238 | 12985 | Hs.2233 | 1440 | Mm00438334_m1 |
| Mu_MIP-2 | MIP-2 | Cxcl2 | CXCL1 | Mm.4979 | 20310 | Hs.789 | 2919 | Mm00436450_m1 |
| Mu_IL-6 | IL-6 | Il6 | IL6 | Mm.1019 | 16193 | | | Mm00446190_m1 |
| Mu_TRANCE | TRANCE | Tnfsf11 | TNFSF11 | Mm.249221 | 21943 | Hs.333791 | 8600 | Mm00441908_m1 |
| Mu_MCP-5 | MCP-5 | Ccl12 | CCL12 | Mm.867 | 20293 | | | Custom Design |

Reactions were performed in triplicate assays using representative RNA samples derived from three pools of five aortas as described previously (45-47).

Results

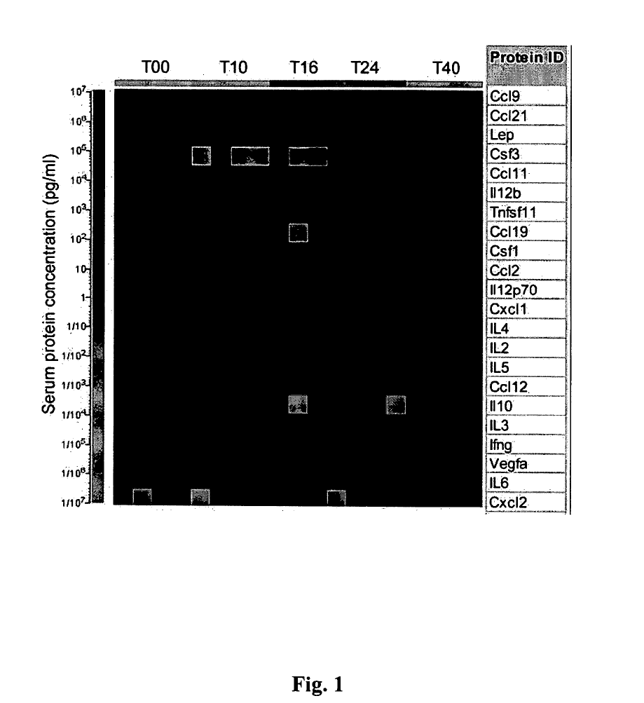

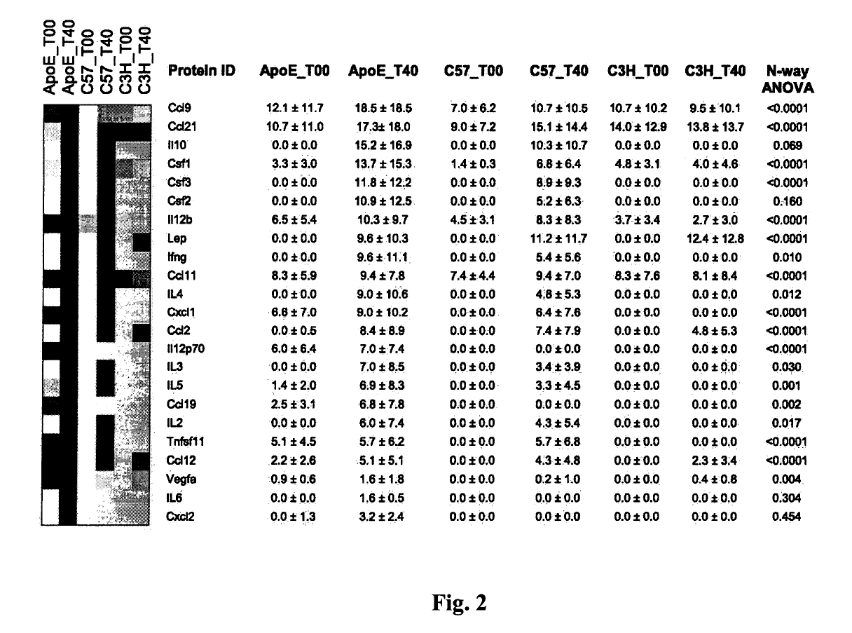

Temporal patterns of protein expression during atherogenesis in apoE-deficient mice. We have demonstrated previously (45) the extent of atherosclerotic lesions in this cohort of apoE-deficient mice. Given the extensive atherosclerotic lesions in the aorta as well as the aortic valve of the apoEdeficient mice, other vascular beds were not examined in these studies. To identify serum markers that correlate with the extent of atherosclerotic lesions, we have utilized a protein microarray to simultaneously measure the serum level of 30 inflammatory markers in apoE-deficient mice on a high-fat diet throughout the time course of disease development. For control groups, we utilized the apoE-deficient mice on normal diet as well as wild-type C57B1/6J and C3H/HeJ mice at two time points. Eight out of the thirty markers measured did not reveal significant serum expression levels. Twenty-two markers revealed unique time-related patterns of expression, some of which closely correlated with the extent of atherosclerotic lesions in the aorta previously described in this cohort of mice (FIG. 1) (45). These markers included various chemokines (Ccl2, Ccl9, Ccl11, Ccl19, Ccl21, Cxcl1, and Cxcl2) and several cytokines (Il2, Il4, Il5, Il6, Il10, and Il12) as well as other inflammatory proteins (Csf1, Csf2, Csf3, Ifng, Tnfsf11) and Vegfa. The vast majority of these markers had higher expression in apoE-deficient mice compared with control wild-type C57B1/6J and C3H/HeJ mice (FIG. 2). As described previously, under similar conditions, the control mice did not develop histologically evident atherosclerotic lesions (47); therefore, disease-related changes can be readily distinguished from other factors such as high-fat diet and aging.

Strain-specific protein expression with high-fat diet and aging. To account for atherosclerosis-independent variation in serum protein levels due to high-fat diet, aging, and genetic background, we used a number of controls including two previously well-studied mouse strains with different propensities to develop atherosclerosis, two different diets, and a longitudinal experimental design. We have shown previously that these control mice did not develop atherosclerotic lesions and thus were appropriate controls to account for these independent variables and possible interactions among them. As a result, we were able to identify differentially expressed proteins that are likely to be related to each variable and distinguish those specifically related to vascular disease processes in the apoE-deficient model. Simple ANOVA revealed at least 12 markers that were differentially expressed among the various diet-strain-time combinations (FIG. 2). To account for possible interactions among the three independent variables, we utilized three-way ANOVA. Three independent variables have three first-order interactions (time-strain, time-diet, strain-diet) and one second order interaction (time-strain-diet). Accounting for interactions among all three factors, we identified five proteins as differentially expressed (3-way ANOVA, P<0.05), including Ccl9, Ccl21, Ccl11, Csf1, and Il12b.

At the later time points, the high-fat diet also stimulated an inflammatory response in C57B1/6 wild-type mice, as represented by elevated serum levels for a number of inflammatory markers (FIG. 2). C3H/HeJ mice, on the other hand, had the lowest levels of inflammatory markers, even when on the high-fat diet. This finding is consistent with observations from our prior study comparing the aortic vascular wall gene expression in C3H/HeJ mice with that of C57B1/6J mice. That study concluded C57B1/6J mice have a higher genetic propensity for the expression of inflammatory markers in atherosclerosis.

Identification of time-specific protein expression signature pattern in mouse serum. Classification approaches to human cancer have provided significant insights regarding the clinical features of the tumor, including propensity to metastasis, medication responsiveness, and long-term prognosis (13, 23, 33, 43). For atherosclerosis, the clinical utility of classification algorithms will be in prediction of future events. In a previous study, we have applied classification algorithms to establish a panel of genes whose expression in the vessel wall could accurately classify disease severity in atherosclerotic vascular tissue derived from both mice and humans (45). In the current study, we have employed a similar approach to identify a minimal subset of serum proteins to accurately classify each proteomic experiment with one of the four defined stages of atherosclerosis in mice (FIG. 3). Here we utilized several well-known classification algorithms to identify the variables that can best distinguish between the mice with different disease states. These algorithms included RFE, SVM, and ANOVA. We also used PAM as an additional classification algorithm. These algorithms rank the proteins based on their utility for class discrimination between time points of 0, 10, 24, and 40 wk in apoE mice on high-fat diet. Our results demonstrated that a small subset of proteins (Ccl21, Ccl9, Csf3, Tnfsf11, Vegfa, Ccl11, Ccl2) were identified by a majority of the algorithms (FIG. 3A).

The predictive power of the signature pattern of this panel was superior to any single marker, since no individual marker was able to accurately classify the various disease states (analysis not shown). To determine the utility of serum levels of these proteins for classification of mice with different disease states, we utilized the SVM algorithm (linear kernel) to generate a confusion matrix using cross-validation with repeated splits into 75% training and 25% test sets. This algorithm demonstrated that the signature pattern of expression of these serum proteins can distinguish groups of mice with and without disease with up to 100% accuracy (FIG. 3B). Mice with intermediate stages of the disease are also distinguished from the other stages with a high degree of accuracy (79.6-100%) (FIG. 3B).

Cross-validation and analysis of independent data sets. A key proof of the utility of a defined set of classifier proteins is their ability to correctly classify data from an independent experiment. To validate the utility of the classifier proteins, we investigated their ability to accurately categorize an independent group of 16-wk-old apoE-deficient mice. Using the SVM classification algorithm, we were able to accurately classify each of the replicate experiments with the correct stage of the disease process (FIG. 3C). As indicated by the greatest correlation between protein expression in this independent group of mice and protein expression patterns in the original experimental group, aged 10 wk, the classifier proteins accurately matched this validation data set to the closest time point in the training set. It is important to note that, in this analysis, the independent data set (“test”) was not included in the training set (“known”).

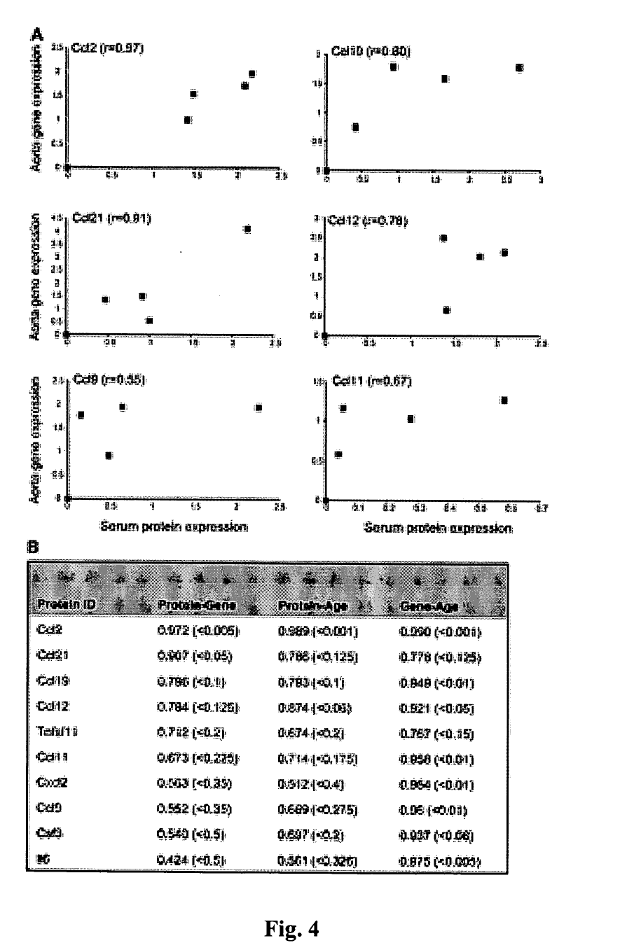

Biomarker serum protein levels correlate with vascular wall gene expression levels. Those biomarkers whose circulating protein levels correlate with molecular events and expression levels in the vessel wall are expected to be most informative about vascular disease. To investigate such correlations, and to gain insights from the biomarker data regarding the pathophysiology of atherosclerosis, we have investigated vascular wall gene expression patterns for genes encoding informative biomarkers. Using quantitative real-time RT-PCR, we were able to correlate serum protein levels of several markers with their vascular RNA expression. Among the markers studied, Ccl21 (r=0.91), Ccl2 (r=0.97), Ccl19 (r=0.80), and Ccl11 (r=0.67) revealed a remarkably high correlation between time-related increase in gene expression and in serum levels (FIG. 4). Although these data do not exclude expression of these markers in other tissues, they suggest that expression is particularly associated with the atherosclerotic vascular wall. Pearson correlation values were determined comparing normalized average ratios of serum protein level, vascular gene expression, and time on high-fat diet (log10 of no. of wk on diet). A correlation coefficient (r) between mRNA expression in an atherosclerotic vessel wall and serum levels of the encoded protein are considered significant if r is at least 0.6; at least 0.7; at least 0.8; at least 0.9, or higher.

Discussion

There is an obvious need for improved tools to diagnose and treat preclinical atherosclerosis. At present, although insights into mechanisms and circumstances of atherosclerosis are increasing, our methods for identifying the high-risk patients and predicting the efficacy of measures to prevent coronary artery disease are still inadequate. Because of a lack of highly sensitive and specific biomarkers for atherosclerotic disease, the first clinical presentation of more than one-half of these patients is either myocardial infarction or death (19, 20). Several inflammatory markers have been studied in the context of atherosclerosis, both in mice and humans, and the results have strengthened the inflammatory hypothesis of atherosclerosis (38). However, each study has focused on only a few individual markers, some lack longitudinal design, and only a few demonstrate direct correlation with gene expression at the vascular level (25, 29, 34).

Currently, the general markers of inflammation, although proposed for use in risk stratification of patients with atherosclerotic disease, are not used in the screening of asymptomatic patients for accurate disease classification and, more importantly, for prediction of first cardiovascular events. The lack of specificity of markers such as C-reactive protein (CRP) and fibrinogen may stem from the fact that they are not derived from the vasculature and may signal inflammation in any organ. It is also possible that, because of heterogeneity among the population at risk, a single marker cannot provide sufficient information for accurate prediction of disease. For similar reasons, these general markers of inflammation such as CRP and sedimentation rate (ESR) have been long abandoned as specific diagnostic markers in other inflammatory diseases such as lupus (SLE) and rheumatoid arthritis (RA).

We have shown previously with RNA profiling studies of mouse aortic tissues, with the same experimental design as that used here, that it is possible to identify a small number of genes capable of classifying disease severity (45). Obviously, given that the vascular tissue is not readily accessible, identification of protein markers in the serum can have practical implications in developing diagnostic tools for diagnosis of coronary artery disease in humans. In the work reported here, we have investigated inflammatory serum biomarker abundance patterns and whether a subset of these biomarkers can be used to classify animals with respect to disease progression. Scientifically, these two types of information are complementary and provide significantly greater insights into the detailed molecular mechanisms of the disease, from gene transcription to translation to intracellular pathways to secretion of mediators into the serum. As noted above, identification of the serum marker profile for a given disease state allows the development of noninvasive diagnostic approaches that can be used in humans. Because we also have a detailed microarray-based picture of the transcriptional landscape in the diseased tissue, we can use this view to assess upstream components in the pathways that lead to inflammatory mediator expression, the first step in developing highly targeted therapeutics. Indeed, serum assays such the one described here can then be used to assay the ultimate effects of such therapeutics. We utilized protein microarrays for simultaneous protein expression profiling of sera from various mouse models of atherosclerosis with different susceptibilities and severities of atherosclerosis. Using classification algorithms similar to those utilized in classifying cancer progression and type, we were able to show that the unique signature patterns of these vascular-derived biomarkers could accurately predict different severities of atherosclerotic disease in mice.

In the prior study (45), our analysis revealed that the microarray gene expression profile of the independent data set derived from the 16-wk time point associated more closely with the 24-wk time point, whereas, in the present study, the protein profiles of the similar time point correlated more closely with the 10-wk time point. This finding may offer a number of interesting hypotheses. Given the limited number of probes in the current protein microarray, the protein classifiers in the current study are different from the gene classifiers identified in the prior study. It is also possible that time-related increase in serum protein expression lags behind changes at the level of vascular wall gene expression.

Because there may not be a direct correlation between vascular gene expression and serum protein levels for the same markers because of various factors such as posttranscriptional modification and protein stability, an important validation of these data was the demonstration of disease-related vascular gene expression for a subset of these markers. We show a correlation between the time-related serum levels of these markers and their gene expression in the vessel wall. The time-dependent correlation of disease progression and vascular gene expression suggests that the primary site of marker production is the vessel wall. However, the vasculature may not be the sole source of the inflammatory markers, and it is possible that other tissues such as muscle, spleen, adipose tissue, or liver may contribute to the serum levels of these markers, as suggested by previous reports (22). One marker evaluated in our studies, Il6, is known to be produced in muscle and liver as well as the vascular wall. Interestingly, the serum abundance of Il6 did not correlate with the temporal development of disease, correlating only weakly with gene expression in the vascular wall. These findings suggest that other tissues may contribute to serum levels of some markers, such as Il6, but that the levels of these were not correlated with the disease state studied and do not contribute to the classification panel.

The serum level of some of the systemic inflammatory markers may also be confounded by differences in metabolic parameters among the various mice studied. It has been demonstrated that a high-fat diet stimulates an inflammatory response in the liver (22). The level of expression of these genes remains high throughout the high-fat feeding period. We controlled for these systemic effects by comparing mice fed high-fat diets during both the early and late atherosclerosis stages, so that serum lipid levels are constant (14) but the degree of atherosclerosis changes. These metabolic parameters therefore have a poor correlation with the serum level of markers which demonstrate a linear increase with time. Thus temporal changes in vascular-derived marker serum levels correlate more closely with the degree of atherosclerosis and not lipid levels.

The markers identified in this study provide strong support for the inflammatory nature of atherosclerosis, and the individual markers identified offer some insights into the underlying mechanisms of the disease in mice. These markers include important chemokines specific for both macrophages and T cells. Ccl21 (originally Exodus-2/SLC/6Ckine/TCA4) is the most powerful chemoattractant yet identified for T cells and plays an important role in T cell adhesion and trafficking from the vasculature to tissue sites of inflammation (30). Related chemokines Cxcl2 and Ccl19, also expressed at high levels in our experiments, mediate the firm adherence of T cells to the endothelium by stimulating lymphocyte function-associated antigen-1 (LFA-1) (6, 15). Importantly, Ccl21 is not thought to play a role in T cell effector function during a normal immune response but has been found to be highly induced in endothelial cells in T cell-mediated autoimmune diseases (8). Therefore, the novel finding of disease-related high-level circulating Ccl21, and highly correlated expression of CCL21 in the diseased vessel wall, raises the question of whether autoimmune pathways may play a role in the development of atherosclerosis in mice (44). Ccl21 levels in human disease remain to be measured. Ccl19 [macrophage inflammatory protein (MIP)-3b] has a somewhat similar function to Ccl21. It binds the same receptor, Ccr7, and is a potent chemoattractant for both T cells and B cells. But unlike Ccl21, it appears to also play a role in normal T cell function. Its expression in the atherosclerotic vasculature and the high correlation between serum levels and aortic gene expression are both novel findings.

The roles of Ccl2 (Mcp1 or JE) (3) and Ccl11 (Eotaxin) (10, 17) in atherosclerosis are well established and confirm our findings. We have also documented that the serum levels of both Cxcl2 (MIP-2) and Cxcl1 (KC) are elevated in sera of atherosclerotic mice, consistent with serum levels described by other investigators (29). As was described in that study (29), we found levels of Cxcl2 (MIP-2) to be less reliable. Moreover, given the lower correlation of serum levels with aortic gene expression, it appears that significant amounts of Cxcl2 may be produced by nonvascular tissues, confirming previous observations (29). Nonetheless, we found that the correlation with vascular gene expression of Cxcl2 was still better than other markers such as Il6 and Csf3. Despite the increased levels of Cxcl1 (KC), we did not find this marker to be a consistent predictor of disease, which is consistent with a recent study (34). Vegfa has recently been described as an independent predictor of acute coronary syndrome (18, 24). Our study supports Vegfa as a reasonable classifier in at least three of the algorithms used, confirming its potential utility in monitoring human disease. Another very interesting finding in our study is the role of Tnfsf11 (TRANCE) in atherosclerosis. Tnfsf11 is a member of tumor necrosis factor (TNF) cytokine family and a ligand for osteoprotegerin which functions as a key factor for osteoclast differentiation and activation. This protein is also known to be a dentritic cell survivor factor and is involved in the regulation of T cell-dependent immune response. Osteoprotegerin has recently been identified as a potential risk factor for progressive atherosclerosis and cardiovascular disease in humans (21, 37). Other cytokines that have been speculated to play a role in atherosclerosis include Il12b (25) and Il5 (9). Although we demonstrated their serum level to be predictive of disease state, we failed to confirm vascular-specific expression of Il12b in atherosclerotic lesions.

In summary, the top serum protein classifiers identified in our study encompass a wide range of atherosclerotic biological processes including macrophage chemoattraction (Ccl9, Ccl2), T cell chemokine activity (Ccl21 and Ccl19), innate immunity (I15), vascular calcification (Tnfsf11), angiogenesis (Vegfa), and high fat-induced inflammation (Cxcl1 and possibly leptin). The signature pattern derived from simultaneous measurement of these markers, which represent diverse atherosclerosis-related biological processes, will likely add to the specificity needed for diagnosis of atherosclerotic disease. Further validation of this approach with appropriate prospective trials in human subjects has lead to improved screening diagnostic tools in atherosclerosis and coronary artery disease, as described in Examples 3 through 12, below.

### REFERENCES

1. Fact Book Fiscal Year 2003. Bethesda, Md.: National Heart, Lung, and Blood Institute, 2003.
2. Morbidity and Mortality Chartbook, 2002. Bethesda, Md.: National Heart, Lung, and Blood Institute, 2002.
3. Aiello R J, Bourassa P A, Lindsey S, Weng W, Natoli E, Rollins B J, and Milos P M. Monocyte chemoattractant protein-1 accelerates atherosclerosis in apolipoprotein E-deficient mice. Arterioscler Thromb Vasc Biol 19: 1518-1525, 1999.
4. Burges C J C. A tutorial on support vector machines for pattern recognition. Data Mining Knowledge Discov 2: 121-167, 1998.
5. Bursill C A, Channon K M, and Greaves D R. The role of chemokines in atherosclerosis: recent evidence from experimental models and population genetics. Curr Opin Lipidol 15: 145-149, 2004.
6. Campbell J J, Hedrick J, Zlotnik A, Siani M A, Thompson D A, and Butcher E C. Chemokines and the arrest of lymphocytes rolling under flow conditions. Science 279: 381-384, 1998.
7. Chen M M, Ashley E A, Deng D X, Tsalenko A, Deng A, Tabibiazar R, Ben-Dor A, Fenster B, Yang E, King J Y, Fowler M, Robbins R, Johnson F L, Bruhn L, McDonagh T, Dargie H, Yakhini Z, Tsao P S, and Quertermous T. Novel role for the potent endogenous inotrope apelin in human cardiac dysfunction. Circulation 108: 1432-1439, 2003.
8. Christopherson K W 2nd, Hood A F, Travers J B, Ramsey H, and Hromas R A. Endothelial induction of the T-cell chemokine CCL21 in T-cell autoimmune diseases. Blood 101: 801-806, 2003.
9. Daugherty A, Rateri D L, and King V L. IL-5 links adaptive and natural immunity in reducing atherosclerotic disease. J Clin Invest 114: 317-319, 2004.
10. Economou E, Tousoulis D, Katinioti A, Stefanadis C, Trikas A, Pitsavos C, Tentolouris C, Toutouza M G, and Toutouzas P. Chemokines in patients with ischaemic heart disease and the effect of coronary angioplasty. Int J Cardiol 80: 55-60, 2001.
11. Feezor R J, Baker H V, Xiao W, Lee W A, Huber T S, Mindrinos M, Kim R A, Ruiz-Taylor L, Moldawer L L, Davis R W, and Seeger J M. Genomic and proteomic determinants of outcome in patients undergoing thoracoabdominal aortic aneurysm repair. J Immunol 172: 7103-7109, 2004.
12. Glass C K and Witztum J L. Atherosclerosis. The road ahead. Cell 104:503-516, 2001.
13. Golub T R, Slonim D K, Tamayo P, Huard C, Gaasenbeek M, Mesirov J P, Coller H, Loh M L, Downing J R, Caligiuri M A, Bloomfield C D, and Lander E S. Molecular classification of cancer: class discovery and class prediction by gene expression monitoring. Science 286: 531-537, 1999.
14. Grimsditch D C, Penfold S, Latcham J, Vidgeon-Hart M, Groot P H, and Benson G M. C3H apoE(_/_) mice have less atherosclerosis than C57BL apoE(_/_) mice despite having a more atherogenic serum lipid profile. Atherosclerosis 151: 389-397, 2000.
15. Gunn M D, Tangemann K, Tam C, Cyster J G, Rosen S D, and Williams L T. A chemokine expressed in lymphoid high endothelial venules promotes the adhesion and chemotaxis of naive T lymphocytes. Proc Natl AcadSci USA 95: 258-263, 1998.
16. Guyon I, Weston J, Barnhill S, and Vapnik V. Gene selection for cancer classification using support vector machines. Machine Learning 46: 389, 2002.
17. Haley K J, Lilly C M, Yang J H, Feng Y, Kennedy S P, Turi T G, Thompson J F, Sukhova G H, Libby P, and Lee R T. Overexpression of eotaxin and the CCR3 receptor in human atherosclerosis: using genomic technology to identify a potential novel pathway of vascular inflammation. Circulation 102: 2185-2189, 2000.
18. Heeschen C, Dimmeler S. Hamm C W, Fichtlscherer S, Simoons M L, and Zeiher A M. Pregnancy-associated plasma protein-A levels in patients with acute coronary syndromes: comparison with markers of systemic inflammation, platelet activation, and myocardial necrosis. J Am Coll Cardiol 45: 229-237, 2005.
19. Kannel W B and McGee D L. Epidemiology of sudden death: insights from the Framingham Study. Cardiovasc Clin 15: 93-105, 1985.
20. Kannel W B and Schatzkin A. Sudden death: lessons from subsets in population studies. J Am Coll Cardiol 5: 141B-149B, 1985.
21. Kiechl S, Schett G, Wenning G, Redlich K, Oberhollenzer M, Mayr A, Santer P, Smolen J, Poewe W, and Willeit J. Osteoprotegerin is a risk factor for progressive atherosclerosis and cardiovascular disease. Circulation 109: 2175-2180, 2004.
22. Kim S, Sohn I, Ahn J I, Lee K H, and Lee Y S. Hepatic gene expression profiles in a long-term high-fat diet-induced obesity mouse model. Gene 340: 99-109, 2004.
23. Lapointe J, Li C, Higgins J P, van de Rijn M, Bair E, Montgomery K, Ferrari M, Egevad L, Rayford W, Bergerheim U, Ekman P, DeMarzo A M, Tibshirani R, Botstein D, Brown P O, Brooks J D, and Pollack J R. Gene expression profiling identifies clinically relevant subtypes of prostate cancer. Proc Natl Acad Sci USA 101: 811-816, 2004.
24. Lee S H, Wolf P L, Escudero R, Deutsch R, Jamieson S W, and Thistlethwaite P A. Early expression of angiogenesis factors in acute myocardial ischemia and infarction. N Engl J Med 342: 626-633, 2000.
25. Lee T S, Yen H C, Pan C C, and Chau L Y. The role of interleukin 12 in the development of atherosclerosis in ApoE-deficient mice. Arterioscler Thromb Vasc Biol 19: 734-742, 1999.
26. Libby P. Inflammation in atherosclerosis. Nature 420: 868-874, 2002.
27. Lucas A D and Greaves D R. Atherosclerosis: role of chemokines and macrophages. Expert Rev Mol Med 2001: 1-18, 2001.
28. Luster A D. Chemokines-chemotactic cytokines that mediate inflammation. N Engl J Med 338: 436-445, 1998.
29. Murphy N, Bruckdorfer K R, Grimsditch D C, Overend P, Vidgeon-Hart M, Groot P H, Benson G M, and Graham A. Temporal relationships between circulating levels of CC and CXC chemokines and developing atherosclerosis in apolipoprotein E*3 Leiden mice. Arterioscler Thromb Vasc Biol 23: 1615-1620, 2003.
30. Nagira M, Imai T, Hieshima K, Kusuda J, Ridanpaa M, Takagi S, Nishimura M, Kakizaki M, Nomiyama H, and Yoshie O. Molecular cloning of a novel human CC chemokine secondary lymphoid-tissue chemokine that is a potent chemoattractant for lymphocytes and mapped to chromosome 9p13. J Biol Chem 272: 19518-19524, 1997.
31. Nakashima Y, Plump A S, Raines E W, Breslow J L, and Ross R. ApoE-deficient mice develop lesions of all phases of atherosclerosis throughout the arterial tree. Arterioscler Thromb 14: 133-140, 1994.
32. Napoli C, Palinski W, Di Minno G, and D'Armiento F P. Determination of atherogenesis in apolipoprotein E-knockout mice. Nutr Metab Cardiovasc Dis 10: 209-215, 2000.
33. Paik S, Shak S, Tang G, Kim C, Baker J, Cronin M, Baehner F L, Walker M G, Watson D, Park T, Hiller W, Fisher E R, Wickerham D L, Bryant J, and Wolmark N. A multigene assay to predict recurrence of tamoxifen-treated, node-negative breast cancer. N Engl J Med 351: 2817-2826, 2004.
34. Parkin S L, Pritchett J P, Grimsditch D C, Bruckdorfer K R, Sahota P K, Lloyd A, Overend P, and Benson G M. Circulating levels of the chemokines JE and KC in female C3H apolipoprotein-E-deficient and C57BL apolipoprotein-E-deficient mice as potential markers of atherosclerosis development. Biochem Soc Trans 32: 128-130, 2004.
35. Ramaswamy S, Tamayo P, Rifkin R, Mukherjee S, Yeang C H, Angelo M, Ladd C, Reich M, Latulippe E, Mesirov J P, Poggio T, Gerald W, Loda M, Lander E S, and Golub T R. Multiclass cancer diagnosis using tumor gene expression signatures. Proc Natl Acad Sci USA 98:15149-15154, 2001.
36. Reddick R L, Zhang S H, and Maeda N. Atherosclerosis in mice lacking apo E. Evaluation of lesional development and progression. Arterioscler Thromb 14: 141-147, 1994.
37. Rhee E J, Lee W Y, Kim S Y, Kim B J, Sung K C, Kim B S, Kang J H, Oh K W, Oh E S, Baek K H, Kang M I, Woo H Y, Park H S, Kim S W, Lee M H, and Park J R. The relationship of serum osteoprotegerin levels with coronary artery disease severity, left ventricular hypertrophy and C-reactive protein. Clin Sci (Lond) 108: 237-243, 2004.
38. Ridker P M, Brown N J, Vaughan D E, Harrison D G, and Mehta J L. Established and emerging plasma biomarkers in the prediction of first atherothrombotic events. Circulation 109: IV6-IV19, 2004.
39. Ridker P M, Cannon C P, Morrow D, Rifai N, Rose L M, McCabe C H, Pfeffer M A, and Braunwald E. C-reactive protein levels and outcomes after statin therapy. N Engl J Med 352: 20-28, 2005.
40. Rifai N and Ridker P M. Inflammatory markers and coronary heart disease. Curr Opin Lipidol 13: 383-389, 2002.
41. Ross R. Atherosclerosis—an inflammatory disease. N Engl J Med 340: 115-126, 1999.
42. Saadeddin S M, Habbab M A, and Ferns G A. Markers of inflammation and coronary artery disease. Med Sci Monit 8: RA5-RA12, 2002.
43. Sorlie T, Perou C M, Tibshirani R, Aas T, Geisler S, Johnsen H, Hastie T, Eisen M B, van de Rijn M, Jeffrey S S, Thorsen T, Quist H, Matese J C, Brown P O, Botstein D, Eystein Lonning P, and Borresen-Dale A L. Gene expression patterns of breast carcinomas distinguish tumor subclasses with clinical implications. Proc Natl Acad Sci USA 98: 10869-10874, 2001.
44. Stemme S, Faber B, Holm J, Wiklund O, Witztum J L, and Hansson G K. T lymphocytes from human atherosclerotic plaques recognize oxidized low density lipoprotein. Proc Natl Acad Sci USA 92: 3893-3897, 1995.
45. Tabibiazar R, Wagner R A, Ashley E A, King J Y, Ferrara R, Spin J M, Sanan D A, Narasimhan B, Tibshirani R, Tsao P S, Efron B, and Quertermous T. Signature patterns of gene expression in mouse atherosclerosis and their correlation to human coronary disease. Physiol Genomics 22: 213-226, 2005.
46. Tabibiazar R, Wagner R A, Liao A, and Quertermous T. Transcriptional profiling of the heart reveals chamber-specific gene expression patterns. Circ Res 93: 1193-1201, 2003.
47. Tabibiazar R, Wagner R A, Spin J M, Ashley E A, Narasimhan B, Rubin E M, Efron B, Tsao P S, Tibshirani R, and Quertermous T. Mouse strain-specific differences in vascular wall gene expression and their relationship to vascular disease. Arterioscler Thromb Vasc Biol 25: 302-308, 2005.
48. Tibshirani R, Hastie T, Narasimhan B, and Chu G. Diagnosis of multiple cancer types by shrunken centroids of gene expression. Proc Natl Acad Sci USA 99: 6567-6572, 2002.
49. Wagner R A, Tabibiazar R, Powers J, Bernstein D, and Quertermous T. Genome-wide expression profiling of a cardiac pressure overload model identifies major metabolic and signaling pathway responses. J Mol Cell Cardiol 37: 1159-1170, 2004.
50. Yeang C H, Ramaswamy S, Tamayo P, Mukherjee S, Rifkin R M, Angelo M, Reich M, Lander E, Mesirov J, and Golub T. Molecular classification of multiple tumor types. Bioinformatics 17, Suppl 1: S316S322, 2001.

### Example 2

### Protein Microarray Analysis

To assess the performance of an antibody array of different chemokines (Eotaxin, IP-10, MCP-1, MCP-2, MCP-3, MCP-4, IL-8, MIP1a, and RANTES), we used a commercially available Schleicher and Schuell protein microspot array (FastQuant Human Chemokine, S&S Bioscences Inc., Keene, N.H., US). This array platform utilizes multiple monoclonal highly-specific antibodies spotted onto standard microscope slides coated with a 3-D nitrocellulose surface. with human circulating samples, we chose a group of 11 cases known to have severe coronary artery disease by history and unequivocal positive exercise test or coronary catheterization, and 9 controls with no history and negative exercise or coronary angiogram. Circulating samples were collected and kept frozen at −80C, then thawed immediately prior to use on the array. Each sample was incubated on two replicate arrays. The 11 patient samples and 9 controls were evaluated on a total of 8 slides (8 arrays per slide) made in one print run.

Reproducibility between arrays was good, as evidenced by replicate experiments done for each sample in the study. For each antibody, a median background subtracted signal of 4 replicate features printed on the same array was plotted against each median obtained in the replicate experiment. A correlation coefficient of 0.99 between measurements with replicate experiments was common, indicating excellent agreement between the two sets of array data.

In the analysis that follows, each analyte circulating measurement represents the average of four measurements on a single circulating sample, from which was subtracted corresponding average measurements from the blank slide, and analyses conducted with log(10) values of this difference. Protein levels in the group of 9 control samples were compared to protein levels in the group of 11 cases. For each protein, distribution of protein levels in case and control groups were compared using the Gaussian error score, which measures the overlap of normal distributions fit to values in each group of samples, and graphed as a heat map. The Gaussian plot shows the actual distribution of protein levels in two groups for the MMP-2/TIMP-2 complex. There is not one single protein measurement that can provide clear separation of the small numbers of individuals in these groups, and the overlapping signal distribution is clearly seen with the Gaussian plots. While the goal of this work was not to identify classification algorithms, it was possible to classify case and control samples by combining a small number of the top proteins with Fisher's Linear Discriminant Analysis.

To validate the findings from the array, we used the standard ELISA sandwich format assay, employing the same capture and detection antibodies that are used with the array. Although the antibody pairs used in the array are from commercial sources and have already been validated for ELISA by the supplier, they were checked prior to use in the array to ensure that they were working according to sensitivity specifications. Case and control human circulating samples are analyzed with ELISA methodology, and the ELISA data compared with the array data. The comparative data for one such analyte, circulating leptin showed a good correlation, whether the ELISA was performed on 10-fold or 20-fold dilutions of the samples.

### Example 3

### Signature Pattern of Circulating Inflammatory markers for Accurate Prediction and Diagnosis of Human Coronary Artery Disease

Serum Biomarker Data from Human Pilot Study

Given the encouraging results obtained in Examples 1 and 2, we examined whether protein microarrays can be used to identify signature patterns of serum inflammatory proteins that can serve as highly sensitive and specific markers of atherosclerotic disease in humans. To investigate this approach we designed a nested case-control study by selecting 51 patients with clinically significant CAD and 44 healthy control subjects from a large clinical epidemiological study designed to examine risk factors and genetic determinants of atherosclerosis. Serum samples collected at the time of enrollment were used for simultaneous measurement of multiple inflammatory markers using a protein microarray. Concentrations of a subset of the analytes tested were significantly higher in case subjects. Classification algorithms using the serum expression profile of these markers accurately stratified CAD subjects compared to controls. Moreover, the unique signature pattern of the biomarkers significantly improved the predictive capacity of other known markers of CAD. In this pilot study we were able to demonstrate that a signature pattern of circulating inflammatory markers accurately identifies patients with atherosclerotic disease.

Introduction

Atherosclerotic cardiovascular disease (ASCVD) is the primary cause of morbidity and mortality in the developed world^{1, 2}. However, due to lack of accurate early diagnostic markers, the first clinical presentation of more than half of the patients with coronary artery disease (CAD) is either myocardial infarction or death^{3, 41, 2}. Inflammation has been implicated in all stages of ASCVD and is considered to be the pathophysiological basis of atherogenesis, providing a potential marker of the disease process^{5 6 7}.

Elevated serum inflammatory biomarkers have been shown to stratify cardiovascular risk and assess response to therapy in large epidemiological studies⁸⁹. Although potentially useful in risk stratification, the current inflammatory markers lack sufficient disease specificity to be used as a screening tool in CAD diagnostics. The lack of accuracy of current markers, such as C-reactive protein (CRP) and fibrinogen, may stem from the fact that they are not primarily derived from the vascular wall nor produced primarily by cells involved in the vascular inflammatory process, and may signal inflammation in a number of different organs and tissues. In addition, it is also possible that, due to the heterogeneity of the disease phenotype in the population at risk, a single marker could not provide sufficient information for an accurate assessment of the vascular damage in coronary circulation. For similar reasons, the general markers of inflammation such as CRP and erythrocytes sedimentation rate (ESR) have been long abandoned as specific diagnostic markers in other inflammatory diseases such as lupus (SLE) and rheumatoid arthritis (RA) although they remain tools to risk stratification and response to therapy in clinical practice

Thus, there is a critical need for biomarkers that more accurately reflect ASCVD activity, and can be used as highly sensitive and specific assays for patient identification. We hypothesize that unique signature patterns of circulating inflammatory proteins can be used to better identify individuals with CAD. To address this issue, we designed a nested case-control study by selecting 51 patients with recent myocardial infarction (MI) and 44 healthy control subjects from the ADVANCE Study ((Atherosclerotic Disease, VAscular FuNction, & GenetiC Epidemiology), a population-based study on the genetic susceptibility of atherosclerosis. Using serum samples collected at the time of enrolment, we performed a simultaneous measurement of nine inflammatory markers with a commercially available protein microarray. For data analysis we also included extensive clinical variables such as medical history, medication profile, personal and family history (first degree relatives) as well as plasma glucose, insulin, and C-reactive protein (CRP) levels. Statistical algorithms identified a signature pattern of protein biomarkers that, when used in combination with other clinical variables, accurately classified individuals with CAD and controls.

Methods

Patient Selection and Clinical Data

All study protocols were reviewed and approved by Institution Review Board. Patients were randomly selected from two different groups of the ADVANCE study cohort, a larger genetic epidemiological study conducted in collaboration between Stanford Cardiovascular division and the Northern California Kaiser Permanente Medical Care Program, Division of Research, and designed to investigate the genetic determinants of cardiovascular disease. ADVANCE recruited a total of 3666 individuals in the San Francisco Bay Area, who were stratified based on sex and age to represent the Northern California population. All potential subjects gave written, informed consent to participate and the study protocol was approved by the Human Subjects Committees of both Stanford University and Kaiser Division of Research. The ADVANCE study cohort is structured in well-characterized clinical groups: 743 young, apparently healthy controls (group 1); 1023 older controls (group 2); 503 young CAD cases (group 3); 926 older newly diagnosed CAD cases, with documented first-onset myocardial infarction (MI) at the time of enrollment with median time of event to enrollment of 3.4 months (group 4); and 471 older cases of first-onset stable angina (group 5). From group 2 and 4 we selected a total of 95 Caucasian subjects, 44 MI cases and 51 controls, by gender-stratified random sampling. Extensive ADVANCE study database includes clinical variables such as medical history, medication profile, personal and family history (first degree relatives) as well as plasma glucose, insulin, C-reactive protein (CRP) levels, and lipid profile. Lipid profiles were available in group 2 only. Case subjects included 45-75 years old men and 55-75 women with first presentation of CAD as an acute MI. These subjects were identified by presence of a primary hospital discharge diagnosis code of 410.x and elevated cardiac enzymes during hospitalization or within 72 hours prior to admission (either troponin I level≧4.0 ng/mL or, at least, one elevated value of CK-MB≧5.6 ng/ml or CK-MB %≧3.3 ng/mL). Serum was collected between 7 to 20 weeks after the index event (median 3.4 months). A committee of ADVANCE study investigators reviewed the clinical documentation to confirm the diagnosis. Controls were 60 to 69 years old individuals, of both sexes, without clinical history of any ASCVD manifestation or other major diseases, as reported by their primary care physician and the Kaiser Permanente database. Clinical data and fasting serum specimens were collected during the first visit after enrolment to ADVANCE study. Plasma concentrations of glucose and insulin were measured with standard methodologies. CRP was determined by high-sensitivity ELISA assay.

Protein Microarray Hybridization and Data Processing

To assess the concentrations of 9 different chemokines (Eotaxin, IP-10, MCP-1, MCP-2, MCP-3, MCP-4, IL-8, MIP1a, and RANTES), we used a commercially available Schleicher and Schuell protein microspot array (FastQuant Human Chemokine, S&S Bioscences Inc., Keene, N.H., US). This array platform utilizes multiple monoclonal highly-specific antibodies spotted onto standard microscope slides coated with a 3-D nitrocellulose surface. The sensitivity and specificity of these markers and correlation to conventional ELISA has been demonstrated previously. Lack of cross-reactivity among these markers has been established previously. Plasma samples are hybridized to protein arrays using manufacturer's instructions, followed by addition of a biotinylated secondary antibody and Cy5-streptavidine conjugate. Resulting fluorescence intensity was measured using an Axon Genepix 4000B microarray scanner in conjunction with a feature extraction software (Array Vision Fast 8.0, S&S Biosciences) to convert the scanned image into numeric intensities. Absolute concentrations were measured by interpolation of intensity values with internal standard references run in parallel. Fast Quant protein arrays present control variability ranging from 3 to about 15% and sensitivity from 1 to 10 pg/ml, depending on the specific analyte. Accuracy of FastQuant protein arrays are comparable to the correspondent ELISA determinations^{10, 11} with a similar linear range. Detailed supplemental methods and quality control results for the current study are provided online on publisher's website (see supplemental materials for Ardigo, Tabibiazar, et al., “Signature Patterns of Circulating Biomarkers Accurately Predict Presence of Coronary Artery Disease”), including array reproducibility and standard curves.

Numerical raw data were subsequently both analyzed in local Windows workstations and migrated into an Oracle relational database specifically designed for microarray data analysis. For technical reasons, RANTES and IL-8 were discounted from further analysis. The RANTES standard curve was non-sigmoidal and, therefore, did not have a linear portion for calculating concentrations. In both case subjects and control samples, most of the IL-8 values were outside the standard curve limits.

Statistical Analysis

Differences in clinical characteristics between the two groups were investigated using Mann-Whitney's U and Chi-square tests, for continuous and nominal variables respectively. The level of significance was computed by Monte Carlo approach. A general linear model (GLM) multivariate analysis was performed to identify differences in chemokines between cases and controls, before and after adjustment for clinical variables unequally distributed between the two groups at U and Chi tests.

The diagnostic performance of chemokines was tested by Receiver Operating Characteristic (ROC) curves.¹² Logistic regression (LR) analysis was used to verify the contribution of chemokine values in the discrimination between cases and controls. Age, gender, and clinical variables significantly different between the two groups in the bivariate analysis were also included into the models as independent variables. Since the difference between the two groups in the intake of medications typically prescribed to CAD patients, such as ACE-inhibitors and statins, would have introduced spurious predictors of disease in the model, we decided to exclude any information about pharmacological treatments from the analysis.

Three different LR models were created to manage the presence of several issues: relatively elevated number of independent variables, presence of missing values (about 10 values in 8 subjects), and co-linearity among chemokine concentrations. A stepwise model, with forward selection of the variables (entry probability 0.05; removal probability 0.15), was performed twice: without and with estimation of the missing values by conditional mean. A third LR model, specifically conceived to address the colinearity issue, included a chemokine score along with the clinical variables. The score computation consisted of recoding each chemokine concentration on a 1 to 10 scale (based on deciles) and then averaging the scale values for any available chemokine values. Full-length description of tests issues, models building process, and estimation procedure for missing values, is available on-line as supplemental material. U and Chi-square tests, GLM, ROC, and LR were performed using SPSS statistical software for Windows, version 12.0 (SPSS Inc., Chicago, Ill.).

To overlook data structure, we performed a two dimensional hierarchical clustering analysis (2D-HC). 2D-HC was built using the open-source software TMev, ver. 3.0 (TM4 suite, The Institute for Genomic Research, Rockville, Md.)¹³. Analysis was conducted using complete linkage and Pearson's correlation as distance metrics. To determine the directions of maximum variance in our data, we employed principal component analysis (PCA) in log2 base.

Protein Selection Algorithms and Disease State Classification:

Protein selection and classification algorithms have been described previously (Tabibiazar 2005 Physiol Genomics. 2005 Jul. 14; 22(2):213-26), incorporated by reference). Briefly, for supervised analyses we utilized a number of classification algorithms to rank genes based on their utility for class discrimination between case and control subjects. The algorithms used in this analysis included Support Vector Machine (SVM)¹⁴ and Recursive Feature Elimination (RFE)¹⁵, a recursive version of SVM in which variables are ranked repeatedly while a fixed fraction of worst scorers are removed each time¹⁶. SVM-RFE was used to determine the optimal number of ranked variables to classify the experiments into their correct groups at minimal error rate. The optimal error rate or misclassification is calculated by 1000-times reiterated cross-validation, with 25% of the experiments as the test group and the rest as the training group. As internal validation for the SVM results we also used the following supervised classification algorithms: Classification and Regression Tree (CART), Linear Discriminant Analysis (LDA), and Logistic Regression (previously described in this section). CART is a flexible hierarchical system of classification by a sequence of binary if-then logical conditions that allows setting the degree of individualization of the results and the proportional cost of misclassification. To get a highly accurate classification, we designed terminal nodes to contain pure subgroups or no more than 5 subjects. A priori information included equal class sizes with equal misclassification costs for each of the two classes. Cross-validation of the results was performed by multiple random permutations of 10% of the subjects.

Results

Clinical Characteristics of the Subjects

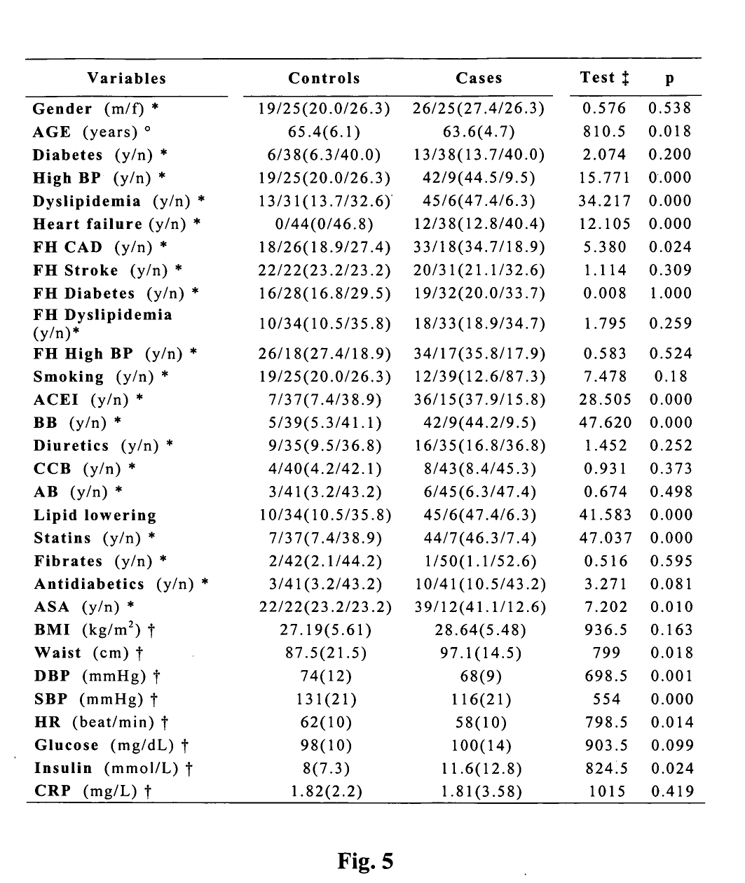

As shown in FIG. 5, the case and control groups differ in a number of important characteristics reflecting well established risk factors for CAD. Case subjects have a more pronounced insulin-resistant phenotype, with higher plasma insulin concentrations, slightly higher BMI (although not significant), larger waist circumference, and increased prevalence of dyslipidemia. However, blood glucose levels and prevalence of diabetes were similar between the two groups. Blood pressure, both systolic and diastolic, was significantly lower in patients than controls, despite a more frequent history of hypertension. This fact can be explained, at least in part, by a greater usage of antihypertensive medications (96.8% vs 43.2%) and medications usually prescribed in secondary prevention, such as ACE-inhibitors, beta-blockers, statins, and aspirin. Moreover, although coronary disease was more prevalent in first degree relatives of CAD patients than controls, family history of diabetes, dyslipidemia, hypertension, and stroke were not significantly different between the two groups. It is interesting to note that, despite a clear difference between the two groups in vascular and metabolic phenotype, no difference in CRP concentration was detectable.

Circulating Inflammatory Markers in Cases and Controls

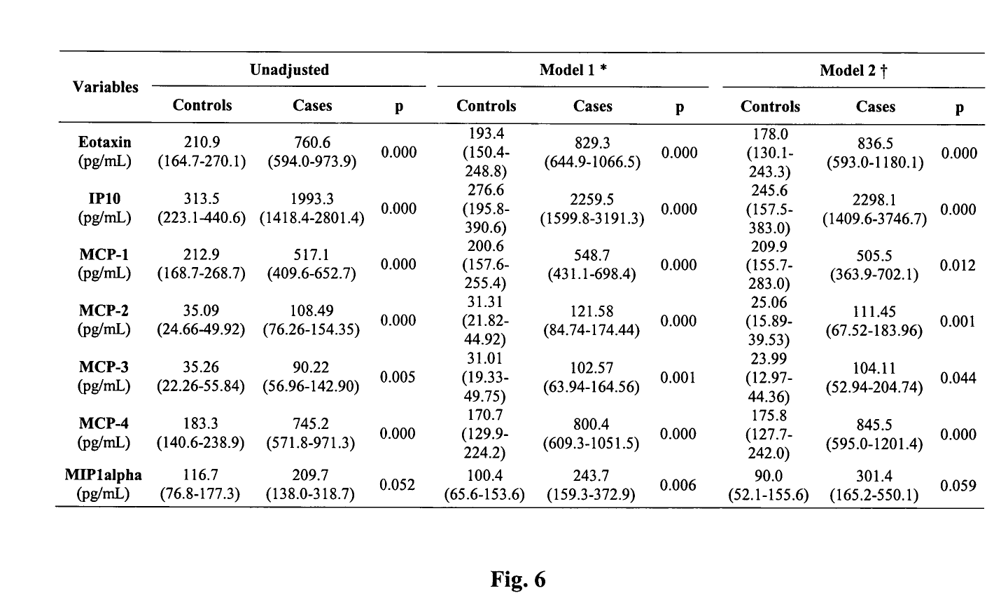

Although CRP was not different between the two groups, multivariate GLM analysis indicated that the other circulating inflammatory markers were higher in cases compared with controls (FIG. 6), even after adjustment for clinical variables and pharmacological therapies.

Unsupervised Data Analysis Comparing Cases vs. Controls

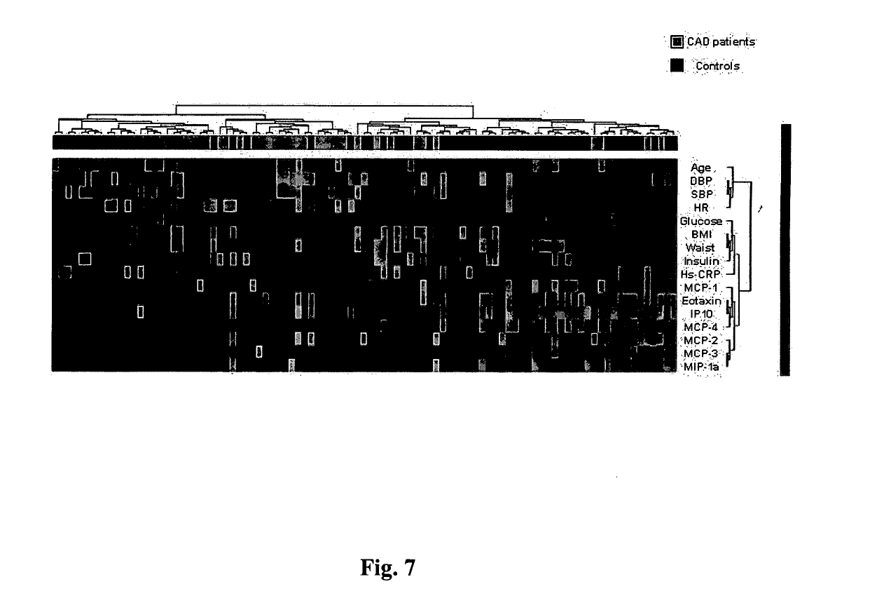

Given increased levels of inflammatory markers in the CAD patients, we studied the feasibility of using that information to accurately cluster patients with unsupervised analysis. Two-dimensional hierarchical clustering indicated that CAD patients and control patients tended to form large homogeneous clusters, although individual cases and controls remained outside these large clusters (FIG. 7). In terms of measured variables, clinical parameters grouped together while chemokines formed a separate cluster. It is interesting to note that CRP levels correlated better to metabolic parameters rather than chemokine levels.

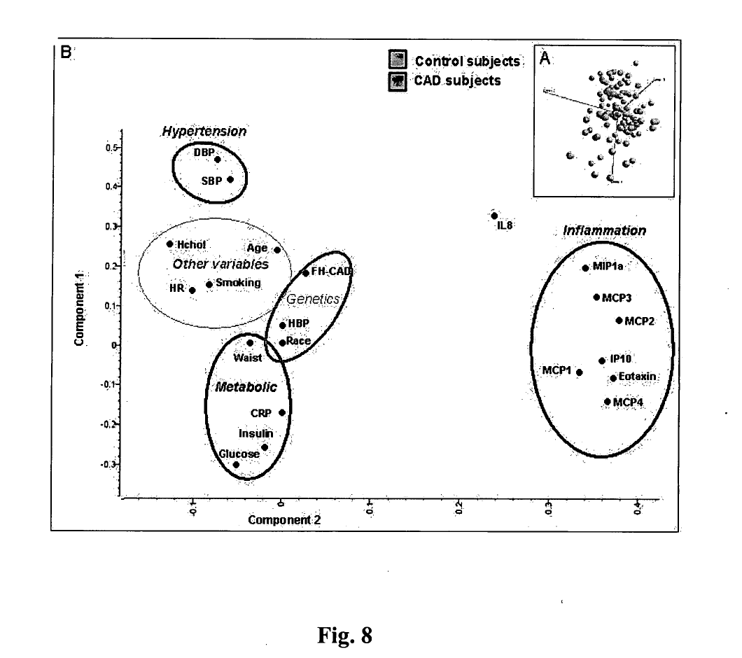

Employing principal component analysis, it was found that 60-70% of the variability observed within the subjects could be explained by chemokines, insulin resistance profile, and a subset of other clinical variables such as hypertension and hyperlipidemia, with markers of inflammation being the dominant factor (FIG. 8).

Classification of Case and Control Status Employing Chemokine Profile and Clinical Variables

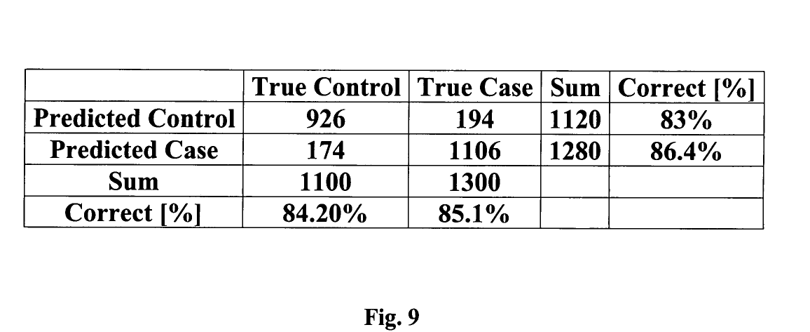

To determine the optimal minimal set of variables that can accurately distinguish between case and control subjects, we utilized the SVM classification algorithm (Tabibiazar 2005 Physiol Genomics. 2005 Jul. 14; 22(2):213-26). SVM identified a set of 15 variables able to stratify subjects with a high degree of accuracy (misclassification rate of <10%) (FIG. 9). In addition to known risk factors for CAD, measurement of circulating chemokines significantly improved the prediction of disease. To validate our findings we employed several other classification algorithms, which yielded similarly high levels of sensitivity and specificity for prediction of CAD: LR (80% sensitivity, 88% specificity), LDA (73%, 94%), and CART (80%, 88%).

Inflammatory Marker Measurements Improve on Classification by Clinical Variables Alone

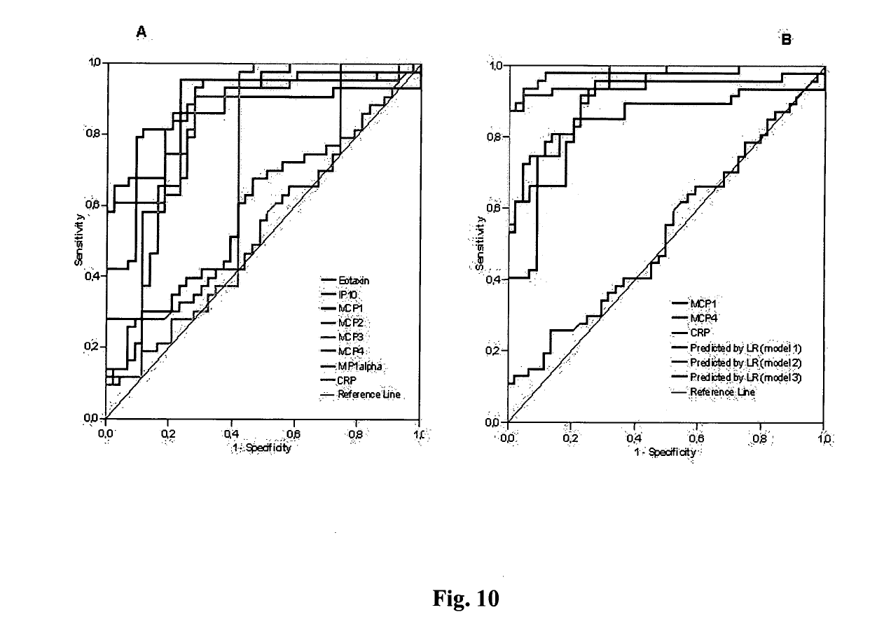

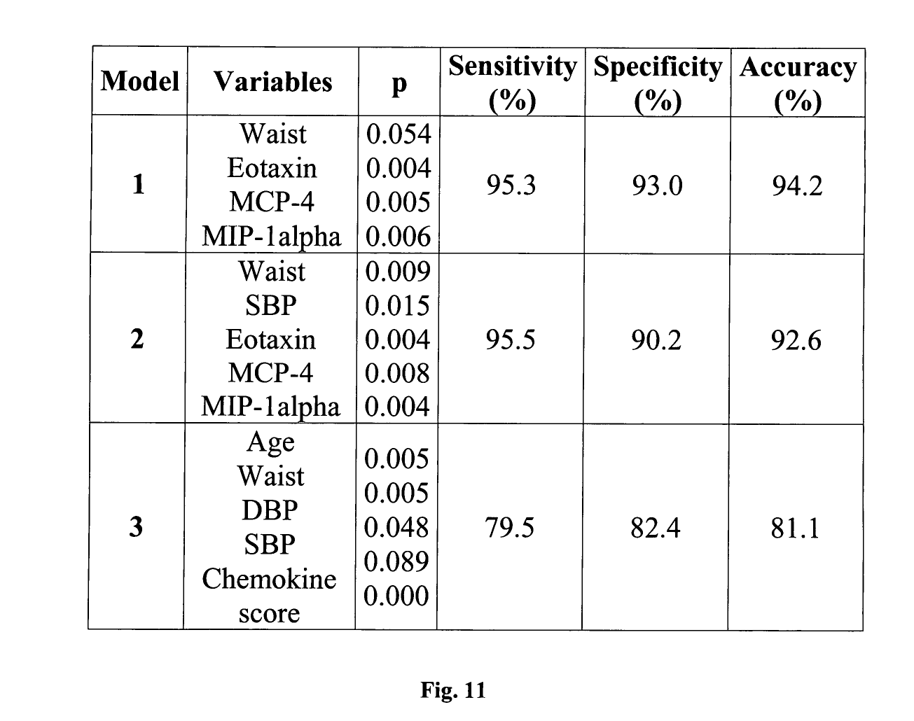

The classification ability of a single versus multiple variables to distinguish case and control subjects was further evaluated using ROC curves. Among the chemokines, MCP-4 appeared to be the most sensitive and MCP-1 the most specific, both showing a good accuracy (AUC 0.896 and 0.849 respectively) (FIG. 10A). It is noticeable that CRP did not appear to be helpful in the identification of disease outside an epidemiologic context, whereas specific markers of vascular inflammation were more accurate. FIG. 11 shows the results of three logistic regression analyses, in which chemokines were entered either by a stepwise selection (models 1 and 2) or as combined score (model 3). Out of three models, two have an overall accuracy in CAD patients over 90%, supporting the hypothesis that the use of multiple markers to distinguish ASCVD patients will be highly informative. Further demonstration is provided by the classification performance of the LR models compared to that of the best chemokines, MCP-1 and -4 (FIG. 10B). It is clear that the use of a multi-marker algorithm provides a better estimate of the presence of disease.

Discussion

There is an obvious need for improved tools to diagnose and treat pre-clinical ASCVD. At present, although insights into mechanisms and circumstances of atherosclerosis are increasing, our methods for identifying high-risk patients and predicting the efficacy of prevention strategies remain inadequate. A growing body of evidence has implicated vascular inflammation as the primary pathophysiological process in every stage of atherogenesis⁵ and several studies have investigated the diagnostic potential of inflammatory markers¹⁷.

Currently, while general markers of inflammation are potentially useful in risk stratification, they are not adequate to identify the presence of CAD in the general population¹⁸. The lack of specificity of these markers may stem from the fact that they are not derived from the vasculature and may signal inflammation in any organ. It is also possible that the heterogeneity of the individual response to environmental risk factors induces a high variability in ASCVD marker concentration. In this context, biological information carried by a single inflammatory protein could be insufficient to provide a comprehensive representation of the vascular inflammatory state, and may not be able to accurately identify the presence and extent of the disease. In contrast, a multidimensional approach utilizing profiles of several inflammatory markers may provide a pathognomonic signature of atherosclerosis-related vascular inflammation. The present study provides experimental support to this hypothesis and suggests that utilization of multiple inflammatory markers may effectively identify patients with coronary heart disease.

Since vascular inflammation is the underlying pathophysiological basis of atherosclerosis, chemokines, which are produced in atherosclerotic vessel, are prime candidates to be markers of CAD. Chemokines are a network of chemotactic proteins produced by white cells and endothelial cells when activated¹⁹. Their main role is accumulation and activation of leukocytes in tissues, and their interaction with several cellular receptors contributes to the specificity of the inflammatory infiltrate^{20,21}. Chemokines are often present as groups with varying composition, and the biological effect of such groups can be quite different from that of individual factors in isolation, so measuring global patterns of cytokine and chemokine expression is more likely to yield biologically relevant information than individual protein assays.

Our data clearly demonstrate that plasma concentrations of several chemokines are differentially regulated in individuals with clinical CAD compared with healthy controls subjects, even after adjusting for known clinical variables. As such, multivariate models combining these markers accurately distinguished samples between the two groups. As hypothesized, prediction models using multiple analytes were much more accurate than those using single inflammatory proteins. These results were validated by several multivariate statistical analyses performed with distinct algorithms yielding remarkably consistent results.

The consistency of each model, as well as the reproducibility of results with different tests, suggests that the chemokine profile represents a strong signal of vascular disease. These results are highly significant despite the relatively small size of the cohort, and the fact that patients were on maximal therapy.

In our data, despite a clear distinction in vascular and metabolic phenotypes, no significant difference in CRP levels was noted between cases and controls. This may be explained by the relatively small sample size as well as the greater use of pharmacological therapies proven to reduce CRP levels, such as statins and aspirin, in the CAD group. However, individuals with previous myocardial infarction remain at higher risk of coronary events than subjects without history of CAD²² despite treatment. Moreover, the major role advocated for CRP in clinical practice is to more accurately stratify individuals when classical risk factors are not definitive, although the issue is still controversial²³. Whereas a decrease in CRP levels during treatment could be used as an index of response to therapy^{8 9}, in our cross-sectional study design, CRP was no more informative than other clinical variables.

There are some limitations to our study. The serum samples from the case subjects were collected post acute event (range 7 weeks to 20 weeks, median 3.4 months). Although inflammatory markers generally tend to return to their baseline levels within 4-8 weeks, we cannot rule out that the acute event can lead to changes in levels of inflammatory markers. Also, our study design does not establish a prognostic value for the proteomic profiles used to distinguish between case and control subjects, although the proteomic profile identified in our study may indeed have a prognostic value for prediction of primary or secondary events. Obviously, our panel of biomarkers is not a comprehensive list. Indeed, the use of a wider array of analytes may improve sensitivity and specificity for diagnosing ASCVD. However, this initial study demonstrates the feasibility of using protein microarrays to simultaneously monitor multiple biomarkers.

In summary, we have identified a panel of circulating serum inflammatory markers whose unique signature patterns can accurately distinguish patients with CAD and controls. A large-scale study validating this approach is reported in Example 5, below.

### REFERENCES

1. NHLBI morbidity and mortality chartbook, 2002. Bethesda, Md.: National Heart, Lung, and Blood Institute, May 2002.; 2002.
2. NHLBI fact book, fiscal year 2003. Bethesda, Md.: National Heart, Lung, and Blood Institute, February 2004.; 2003:35-53.
3. Kannel W B, Schatzkin A. Sudden death: lessons from subsets in population studies. J Am Coll Cardiol. June 1985; 5(6 Suppl):141B-149B.
4. Kannel W B, McGee D L. Epidemiology of sudden death: insights from the Framingham Study. Cardiovasc Clin. 1985; 15(3):93-105.
5. Ross R. Atherosclerosis—an inflammatory disease. N Engl J. Med. Jan. 14 1999; 340(2):115-126.
6. Glass C K, Witztum J L. Atherosclerosis. the road ahead. Cell. Feb. 23 2001; 104(4):503-516.
7. Libby P. Inflammation in atherosclerosis. Nature. Dec. 19-26 2002; 420(6917):868-874.
8. Rifai N, Ridker P M. Inflammatory markers and coronary heart disease. Curr Opin Lipidol. August 2002; 13(4):383-389.
9. Ridker P M, Cannon C P, Morrow D, et al. C-reactive protein levels and outcomes after statin therapy. N Engl J Med. Jan. 6 2005; 352(1):20-28.
10. See manufacturer's information (Whatman; Schleicher & Schuell).
11. See manufacturer's information (Whatman; Schleicher & Schuell).
12. Zweig M H, Campbell G. Receiver-operating characteristic (ROC) plots: a fundamental evaluation tool in clinical medicine. Clin Chem. April 1993; 39(4):561-577.
13. Saeed A I, Sharov V, White J, et al. TM4: a free, open-source system for microarray data management and analysis. Biotechniques. February 2003; 34(2):374-378.
14. Burges C J C. A tutorial on support vector machines for pattern recognition. Data Mining and Knowledge Discovery. 1998; 2(2):121-167.
15. Guyon I, Weston J, Barnhill S, et al. Gene selection for cancer classification using support vector machines. Machine Learning. 2002; 46(1/3):389.
16. Ramaswamy S, Tamayo P, Rifkin R, et al. Multiclass cancer diagnosis using tumor gene expression signatures. Proc Natl Acad Sci USA. Dec. 18 2001; 98(26):15149-15154.
17. Ridker P M, Brown N J, Vaughan D E, et al. Established and emerging plasma biomarkers in the prediction of first atherothrombotic events. Circulation. Jun. 29 2004; 109(25 Suppl 1):IV6-19.
18. Pearson T A, Mensah G A, Alexander R W, et al. Markers of inflammation and cardiovascular disease: application to clinical and public health practice: A statement for healthcare professionals from the Centers for Disease Control and Prevention and the American Heart Association. Circulation. Jan. 28 2003; 107(3):499-511
19. Charo I F, Taubman M B. Chemokines in the pathogenesis of vascular disease. Circ Res. Oct. 29 2004; 95(9):858-866.
20. Sallusto F, Mackay C R, Lanzavecchia A. Selective expression of the eotaxin receptor CCR3 by human T helper 2 cells. Science. Sep. 26 1997; 277(5334):2005-2007.
21. Luster A D. Chemokines—chemotactic cytokines that mediate inflammation. N Engl J Med. Feb. 12 1998; 338(7):436-445.
22. Third Report of the National Cholesterol Education Program (NCEP) Expert Panel on Detection, Evaluation, and Treatment of High Blood Cholesterol in Adults (Adult Treatment Panel III) final report. Circulation. Dec. 17 2002; 106(25):3143-3421.
23. Levinson S S. Brief review and critical examination of the use of hs-CRP for cardiac risk assessment with the conclusion that it is premature to use this test. Clin Chim Acta. June 2005; 356(1-2):1-8.
24. Tabibiazar R, Wagner R A, Ashley E A, King J Y, Ferrara R, Spin J M, Sanan D A, Narasimhan B, Tibshirani R, Tsao P S, Efron B, Quertermous T. Signature patterns of gene expression in mouse atherosclerosis and their correlation to human coronary disease. Physiol Genomics. 2005 Jul. 14; 22(2):213-26.

### Example 4

### Data Analysis for Inflammatory Markers for Accurate Classification of Coronary Artery Disease

A study was undertaken with a commercially available Schleicher and Schuell human chemokine chip. We have employed the array for the evaluation of circulating chemokine levels in 100 samples chosen from the Reynolds Center cohorts. The chemokines measured were: MCP-1, MCP-2, MCP-3, MCP-4, eotaxin, IL-8, RANTES, MIP-1alpha and IP-10, although IL8 and RANTES values fell outside the linear range. Genetic loci encoding MCP-1, MCP-2, MCP-3, eotaxin, IL-8, and RANTES have all been extensively investigated by resequencing and genotyping of chosen SNPs in the Reynolds cohorts. Circulating samples were from fifty individuals with history of myocardial infarction and 50 age-matched controls (see cohort descriptions above). Although the controls were not matched on other variables, there was a similar joint distribution for gender and ethnicity and other variables. Arrays were hybridized with manufacture-supplied reagents, washed, and scanned in an Axon scanner, and feature extraction performed with Schleicher & Schuell proprietary software (ArrayVision™ Quant®). Standard curves were generated with reagents included with the array, and concentrations determined for each circulating sample.

Analyses have taken novel approaches, and have adhered to the basic premise of this proposal, that incorporation of clinical and genotyping data can add information to biomarker data, serving to normalize inter-individual variations of chemokine levels that are not associated with disease status/activity. Analyses were conducted with measurements of chemokine abundance, clinical data, and genotyping information on individual SNPs for the chemokines that had such matching data.

Discriminating between cases and controls, and finding those variables that serve to discriminate, is the fundamental problem of two-class “classification.” While individual classifiers may do well, votes among them typically do even better. Indeed, methods that involve voting among classifiers are popular, two versions being “bagging” and “boosting.” We have begun analyses with only four classifiers, and simple voting among them on a subject-by-subject basis. The standard approach of cross-validation, in particular 5-fold cross-validation, was used to evaluate prospective performance. Thus, the set of data were partitioned at random into five subsets of nearly equal size. Successively, each procedure (and a vote among the procedures) was developed for the 80%, with results computed for the 20%. The five sets of results were then averaged. More sophisticated sample reuse methods may also find use for assessing prospective accuracy.

The cited analyses were undertaken for the preliminary sample of 99 subjects. Variables included eotaxin, IP-10, MCP-1, MCP-2, MCP-4, MIP1alpha, GENDER, AGE, GLUCOSE, INSULIN, CRP, and FAT. The variable FAT was determined as the first principal component of BMI and WAIST, and accounted linearly for 91% of the variability in the two latter predictors. There were 51 MI cases and 48 controls. For purposes of estimating a Bayes classification rule for the two-class problem, we used empirical priors; thus they were almost 0.5 per class. Costs of misclassification were taken to be equal. (Of course, for a two-class problem it is only the ratio of products of prior probabilities and misclassification costs that matter. Here the ratio was about one.) Ages ranged from 60 years to 72 years, with the lower end represented more heavily than the upper. The mean was 64.7 years, with respective 25th, 50th, and 75th percentiles 62, 64, 67; the standard deviation of age was 3.1. In the following examples, LDA refers to Fisher's linear discriminant. Methodologies termed CART, FlexTree and LART are described below. With the LART technology, a simple lasso is used first to reduce the number of predictors. For details of how classification was performed see below. One important detail in both FlexTree and LART is a Hotelling T² sort on regression coefficients that is crucial to their predictive power. Weights that devolve from the sort are used in LART's weighted lasso.

TABLE 3
5-fold cross-validated performance.

| | Percent | | |
|---|---|---|---|
| Algorithm | Misclassified | Sensitivity | Specificity |
| Logistic Regression | 16% | 80% | 88% |
| LDA | 17% | 73% | 94% |
| CART | 15% | 80% | 88% |
| LART | 16% | 78% | 90% |
| Vote | 12% | 82% | 90% |

TABLE 4
Variables identified by the indicated methodology.

| | |
|---|---|
| CART | MCP-4, FAT, eotaxin, MIP1alpha |
| LART | MIP1alpha, MCP-2, MCP-4, eotaxin, AGE, |
| | FAT, Glucose, Insulin |
| Logistic Regression | MIP1alpha, MCP-2, MCP-4, eotaxin |
| LDA | MCP-4, eotaxin, MIP1alpha |

A further analysis incorporated the cited predictors and also information on available SNP genotypes in the same 99 subjects. Five-fold cross-validated percent misclassified decreased to 10%, while sensitivity increased to 85% and specificity to 92%. In this analysis, the simple lasso approach was used to narrow the numbers of SNPs included. Moreover, CART applied to information available on SNPs within a gene was used to impute any missing SNP values.

Overall, these analyses provide compelling support for the invention described herein. Despite the small number of analytes and clinical variables evaluated, a reasonable classification result was achieved, by multiple methods. Circulating chemokine measurements were chosen by all of the methods, and there was overlap between the different methods, with MIP1alpha, MCP-4 and eotaxin featuring in multiple algorithms. These analyses suggest that genotyping data may provide additional useful information. High sensitivity CRP, the current benchmark for atherosclerotic disease was not identified as useful in these classification analyses, suggesting that levels of multiple disease related inflammatory markers may provide significant improvement over existing predictors.

We have summarized the joint distributions of features and of individuals by clustering (unsupervised learning). In our approach to agglomerative, hierarchical clustering (FIG. 6), columns are individuals and rows features. With this algorithm, columns and rows are clustered successively, with the goal of producing sets of features and samples that are “close.” Looking at clustering of variables, it is very informative that the chemokines MCP-2, MIP1-a, MCP-1, IP-10, eotaxin, and MCP-4 all cluster closely together. Also, metabolic variables fasting insulin level, FAT (first principal component of BMI and abdominal girth), and glucose cluster together, as might be expected considering the association of these variables in the context of glucose metabolism and insulin resistance. Gender and age were not found to be close to either of these clusters, and remained separate.

Interestingly, hsCRP did not cluster with the chemokines, but rather the metabolic variables, arguing that hsCRP levels may not track with vascular inflammation as well as a composite chemokine signature. Sample clusters were not homogeneous with regard to class membership, as might be desired. These analyses argue that unsupervised learning (clustering) is not sufficient for doing supervised learning (classification). Based on results thus far, schemes for classification whereby one tries to form groups based not only on features but also on outcome (that are predictive for classifying subsequent observations on the basis of features alone) seem necessary if one is to do accurate classification.

### Example 5

### Large Clinical Trial of 1330 Patients: Signature Patterns of Circulating Biomarkers for Accurate Prediction and Diagnosis of Atherosclerotic Cardiovascular Diseae and Vascular Inflammation

Serum Biomarker Data from a Large Clinical Trial for Validation of Multi-Marker Profiles

Given the encouraging results in the pilot clinical trials, we examined whether multi-marker profiles can be validated in a much larger trial and whether they can serve as highly sensitive and specific markers of atherosclerotic disease in humans. To investigate this approach we utilized a large clinical epidemiological study which included 400 cases of clinically significant ASCVD and 930 control subjects. The study was designed to examine risk factors and other novel determinants of atherosclerosis. Serum samples collected at the time of enrollment were used for simultaneous measurement of multiple inflammatory markers using a protein microarray. Exact methodology used for pilot studies was utilized here (discussed in details in prior examples). Concentrations of a subset of the analytes tested were significantly higher in case subjects. Classification algorithms using the serum expression profile of these markers accurately stratified CAD subjects compared to controls. Moreover, the unique signature pattern of the biomarkers significantly improved the predictive capacity of other known markers of CAD. This larger trial validated our prior finding but also provided with more examples for use of multimarker approach for accurate prediction and diagnosis of atherosclerotic cardiovascular disease and its various clinical sequale.

Prediction of Atherosclerotic Disease: Selection of Informative Markers

The selection of a number of informative markers for building classification models requires the definition of a performance metric and a user-defined threshold for producing a model with useful predictive ability based on this metric. In the following section we will define the target quantity to be the “area under the curve” (AUC), the sensitivity and/or specificity of the prediction as well as the overall accuracy of the prediction model.

Let us now describe one approach for selecting the number of terms for building a predictive model. In this implementation, we will describe the process for selecting markers in the absence of any clinical variables and/or adjusting factors. The process is as follows: We first split randomly our training data into ten groups, each group containing subjects identified as “Healthy” or “Diseased” in proportion to the number of these labels in the complete sample. Each subject was represented by its 24 marker measurements and the label that identifies the state of disease (absent, i.e. “Healthy” of present, i.e. “Diseased”). We chose nine of the groups and for each of the 24 markers: MCP-1, IGF-1, TNFα, IL-5, M-CSF, MCP-2, IP10, MCP-4, IL-3, IFNγ, Ang-2, IL-7, IL-10, Eotaxin, IL-2, IL-4, ICAM-1, IL-6, IL-12p40, MIP1a, IL-5, MCP-3, IL13, IL1b, we trained a model using a given supervised algorithm such as, e.g., Linear Discriminant Analysis, Quadratic Discriminant Analysis, Logistic Regression, etc. on all the data of the 9 groups (i.e. we created a training supergroup). We then applied the model to the tenth group that was excluded from the training procedure and we estimated the testing error “e” and or a number of prediction quality measures described earlier. We repeated the same process 10 times, sampling randomly 9 groups each time for generating a training sample and using the 10^{th} group for estimating the testing error “e” and the prediction quality measures. From the sample of the 10 numbers we then estimated the expected value for each of the prediction quality measures and/or error, as a well the variance of our estimates. Given these values, the marker that improves the average prediction ability of the model as chosen as the first term in the model. We can instead use another measure of improvement instead of the average value of the prediction quality measure, for example we can instead select the term with the highest value of the ratio of the expected quality measure to its variance estimate. Once the first term has been added to the model, we can repeat the process for the remaining markers that did not make it in the current selection step. Thus, in the second step we repeat the aforementioned calculations for the remaining markers. The selection of the second model term can be accomplished by choosing the term that mostly improves our target prediction quality measure or using some combination of the expected value of the current model minus the new model normalized by the errors of those measures.

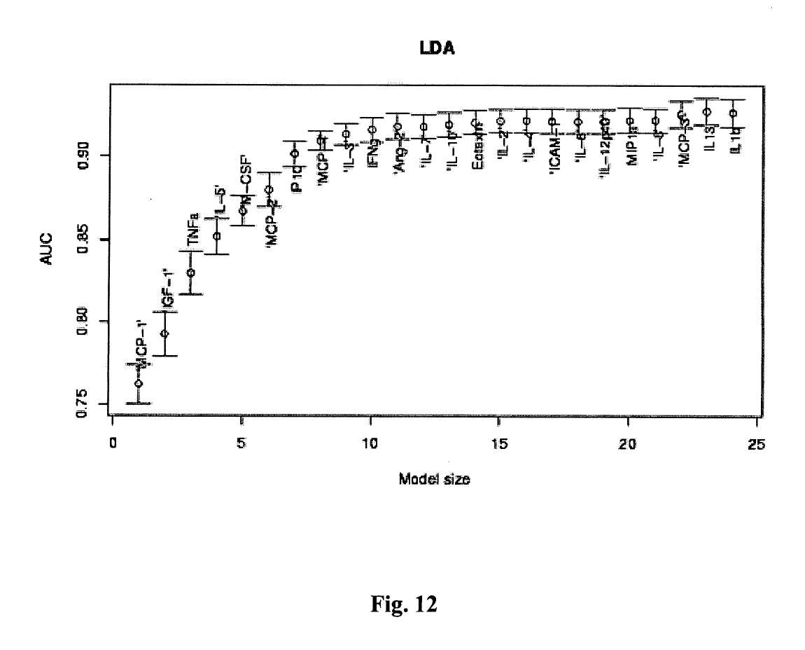

FIG. 12 shows the results of applying this process to a set of 1300 subjects. We selected the threshold of AUC>0.75 as our target prediction quality measure and we selected the terms using a Linear Discriminant Analysis model.

The quality threshold was satisfied using the following marker: MCP-1.

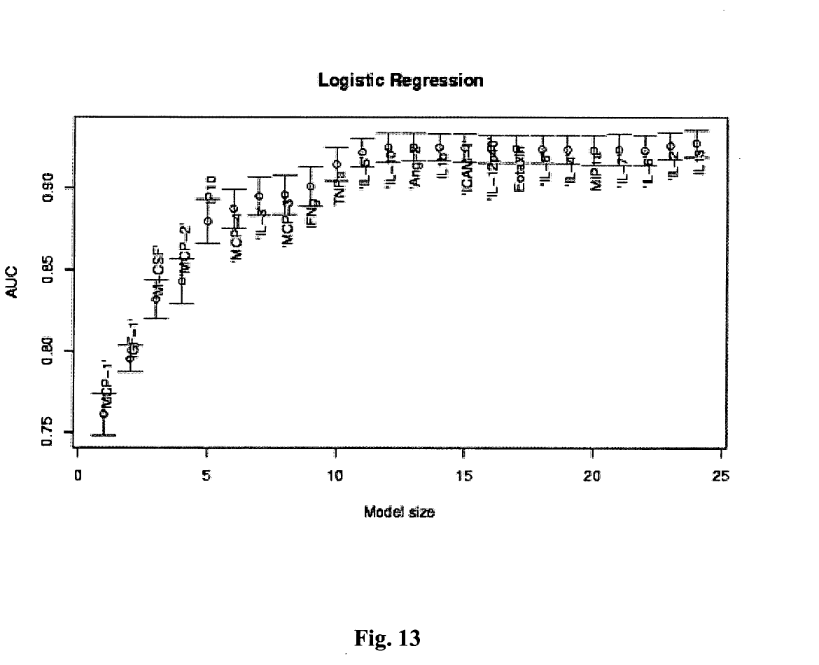

FIG. 13 shows the results of selecting the terms using a Logistic Regression model while keeping the discovery sample and quality thresholds the same. The comparison with the previous example indicates that the two models have only the first two terms in common (MCP-1, IGF-1) but the third term is different (TNFα vs. M-CSF). Thus we can use a combination of markers and predictive models that will exceed our quality measure threshold.

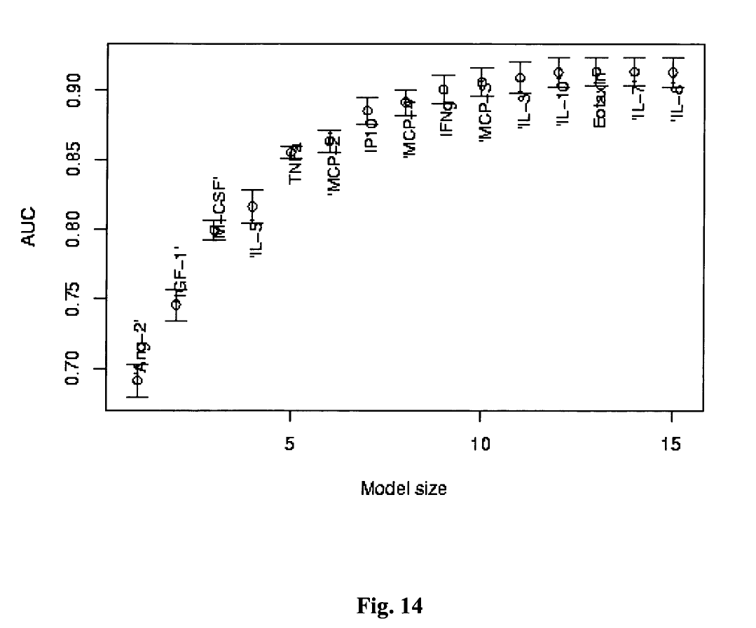

In order to show that we can interchange the markers and still satisfy our requirement for a prediction quality measure, we removed the marker MCP-1 from the pool of available markers for selection and repeated the process. FIG. 14 presents the results of this approach using again an LDA model and the same discovery set of 1300 subjects. The new set of two markers that provide a model with AUC>0.75 is composed of: Ang-2, IGF-1.

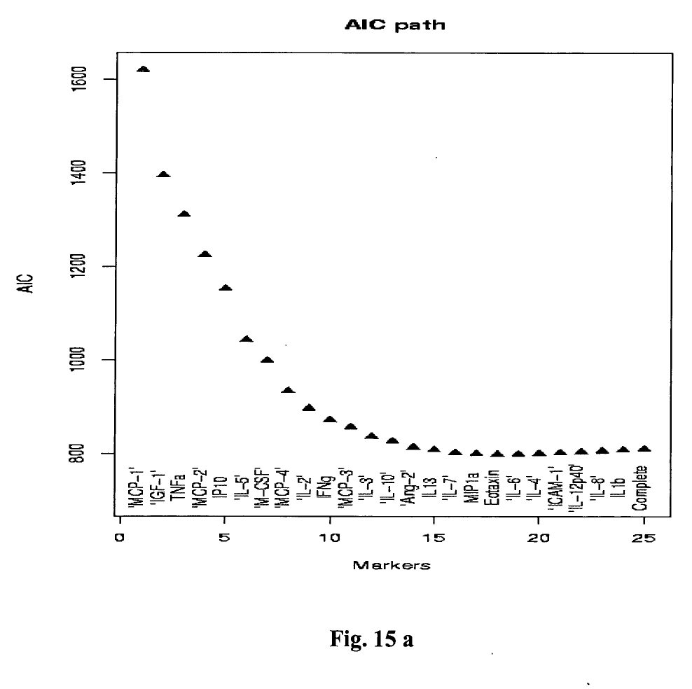

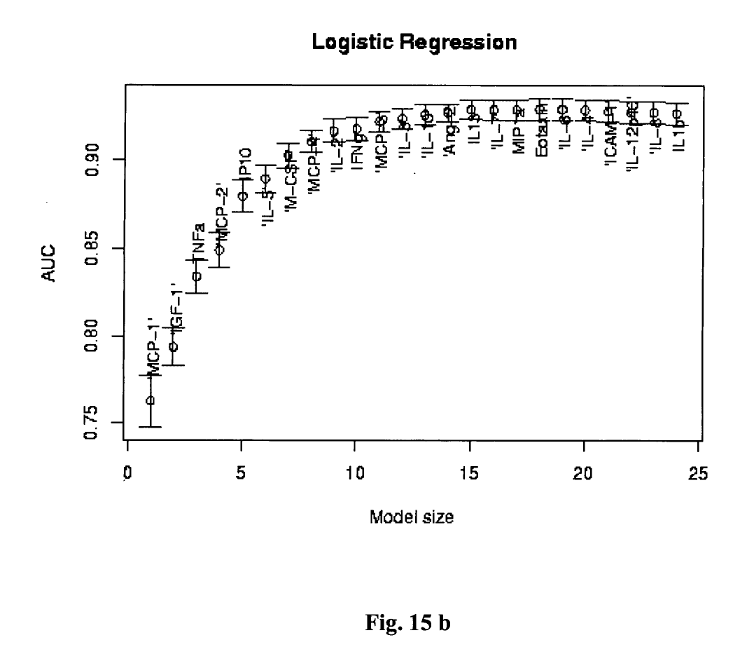

As an example of a different selection criterion, we present the results obtained using the AIC criterion within the framework of a Logistic Regression model. This criterion is usually used in the context of selecting the optimum number of terms for a Logistic Regression model. The criterion balances the error increase due to the removal of a term with the reduction of the number of degrees of freedom that this term contributed to the model. Usually, the process of term elimination starts with the full model and terminates when the removal of a term increases the AIC value. The results of term elimination as a function of the AIC criterion are presented in FIG. 15a (the term elimination process is presented past the optimum point). The AUC predictions for a model incorporating increasing number of terms are presented in FIG. 15b. The addition of terms in the aforementioned model is performed in the reverse order of term removal from the complete model, i.e a model including all 24 markers, that the application of the AIC criterion dictates in the term selection process. The latter approach produces a Logistic Regression model with expected AUC>0.75 using at least one marker (MCP-1).

The process of term selection can be accomplished either with a forward selection (first, second and third examples within this working example) or a backward selection (fourth example within this working example), or a forward/backward selection strategy. This strategy allows for testing of all the terms that have been removed in a previous step in the current reduced model.

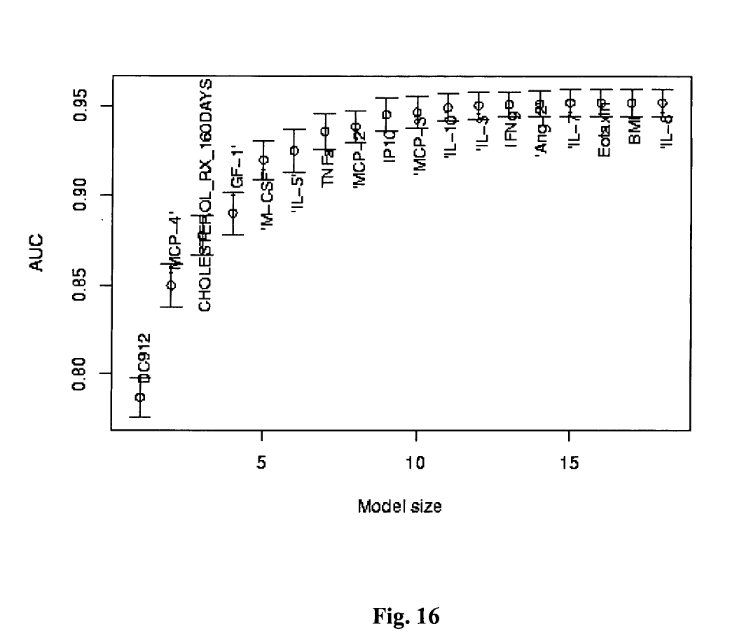

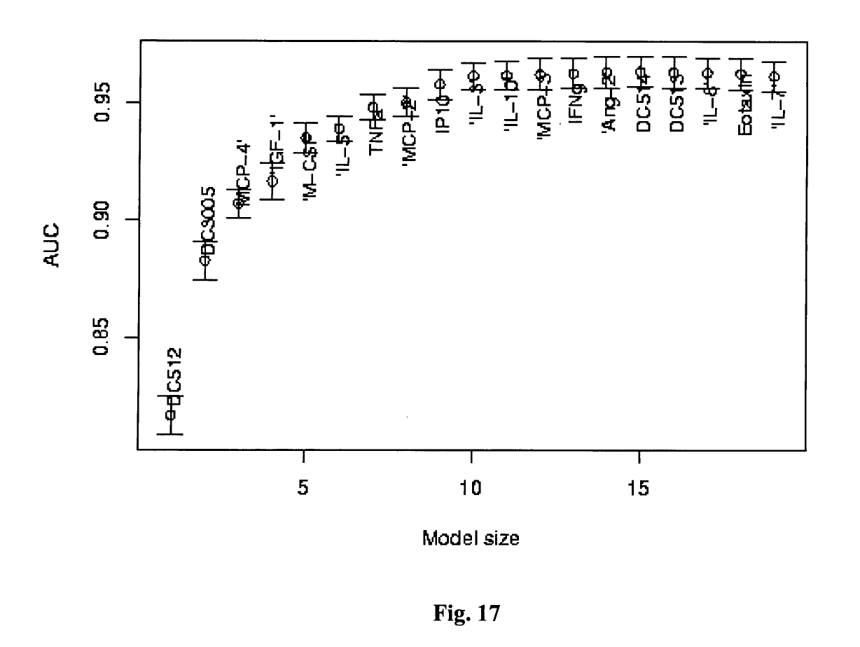

The same selection process can be extended to include both markers and clinical variables. The next two figures, present the results for the case that the candidate variables for a Logistic Regression model include “Hyperlipidemia” (DC912) and “Use of lipid-lowering medication within 160 days before index day” (FIG. 16) or “Statin use,” “ACE blockers use” (FIG. 17) along with all 16 markers. These examples demonstrate that the markers in the set of at least 3 markers required for obtaining an AUC>0.75 can be replaced with clinical variables in the set. The combination of Hyperlipidemia (DC912) and MCP-4 produces a model with expected value of AUC ˜0.85.

Using the aforementioned methods we can also select the number of markers that will optimize the performance of a model without the use of all the markers. One way to define the optimum number of terms is to choose the number of terms that produce a model with average predictive ability (measured as AUC, or equivalent measures of sensitivity/specificity) that lies no more than one standard error from the maximum value obtained for any combination and number of terms used for the given algorithm. Looking back at FIG. 17, a Logistic Regression model that includes the following markers satisfies these requirements: DC512, DC3005, MCP-4, IGF-1, M-CSF, IL-5, MCP-2, IP-10.

### Example 6

### ACE Inhibitor Response Prediction Models

Using the methods described in Example 5, we derived models using Logistic Regression or Linear Discriminant Analysis that classify samples according to the use of ACE inhibitors. These models were adjusted for the status of the subject (Control or Case) since the overall level of the markers depends on whether we deal with a healthy individual or not. The models find use in a variety of methods such as, e.g., screening compounds to identify other agents that act as ACE inhibitors or on convergent pathways, and for monitoring the efficacy of ACE inhibitor therapy. In the first example, the compound is provided to a mammalian subject, one or more samples are taken from the subject and datasets are obtained from the sample(s). The datasets are run through an ACE Inhibitor Response Prediction model and the results are used to classify the sample. If the sample is classified as coming from a subject dosed with an ACE inhibitor, then the compound is likely to be a presumptive ACE inhibitor. In the second example, one or more samples are obtained from a subject and datasets from those samples are run through an ACE Inhibitor Response Prediction model. If the sample is classified as coming from a subject dosed with an ACE inhibitor then the therapy is likely to be efficacious. If multiple samplings over time indicate time dependent changes in the value of a predictor obtained from the model, then the therapeutic efficacy of the medication therapy is likely changing, the direction of the change being indicated by a predictor value trending more toward the medication use classification or the no-medication use classification. The protein markers used in the exemplified models are set out in Tables 5 and 6, below, along with the models' performance characteristics.

TABLE 5
ACE Inhibitor Prediction Model 1.
Logistic Regression

| Variables used: | mis-classification | AUC | sensitivity | specificity | accuracy |
|---|---|---|---|---|---|
| MCP-1, IGF-1, TNFa, MCP-2, IP10, IL-5, M- | 0.365 | 0.688 | 0.641 | 0.632 | 0.635 |
| CSF, MCP-4, MCP-3, IL-3, Ang-2, IL- | | | | | |
| 7, Eotaxin | | | | | |

TABLE 6
ACE Inhibitor Prediction Model 2.
Linear Discriminant Analysis

| Variables used: | mis-classification | AUC | sensitivity | specificity | accuracy |
|---|---|---|---|---|---|
| MCP-1, IGF-1, TNFa, MCP-2, IP10, IL-5, M- | 0.376 | 0.689 | 0.632 | 0.620 | 0.624 |
| CSF, MCP-4, MCP-3, IL-3, Ang-2, IL- | | | | | |
| 7, Eotaxin | | | | | |

### Example 7: ACE Inhibitor or Statin Use Prediction Models

Using the methods described in Example 5, we derived models using Logistic Regression or Linear Discriminant Analysis that classify samples according to the use of ACE inhibitors or statins. These models were adjusted for the status of the subject (Control or Case) since the overall level of the markers depends on whether we deal with a healthy individual or not. The models find use in a variety of methods such as, e.g., screening compounds to identify other agents that act as ACE inhibitors or statins or on convergent pathways, and for monitoring the efficacy of ACE inhibitor or statin therapy. In the first example, the compound is provided to a mammalian subject, one or more samples are taken from the subject and datasets are obtained from the sample(s). The datasets are run through an ACE Inhibitor or Statin Use Prediction model and the results are used to classify the sample. If the sample is classified as coming from a subject dosed with an ACE inhibitor or statin, then the compound is likely to be a presumptive ACE inhibitor or statin. In the second example, one or more samples are obtained from a subject and datasets from those samples are run through an ACE Inhibitor or Statin Use Prediction model. If the sample is classified as coming from a subject dosed with an ACE inhibitor or statin then the therapy is likely to be efficacious. If multiple samplings over time indicate time dependent changes in the value of a predictor obtained from the model, then the therapeutic efficacy of the medication therapy is likely changing, the direction of the change being indicated by a predictor value trending more toward the medication use classification or the no-medication use classification. The protein markers used in the exemplified models are set out in Tables 7 and 8, below, along with the models' performance characteristics.

### Biomarker Profile for Medication use Responsiveness

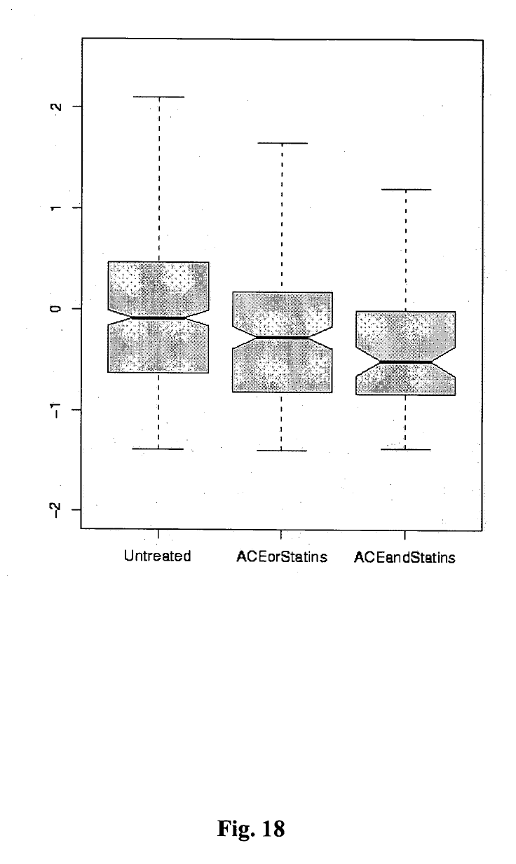

We demonstrate that a panel of markers can be used for monitoring the medication effect on the level of inflammation of a subject. Inspecting the distribution of values for a number of markers (IL-2, IL-5, IL-4) we demonstrate a dosage effect as a function of the number of medications that a control subject is treated with (i.e. no medication vs. one medication vs. two medications). As an example for this approach, we use three medication responsive markers as a panel (IL-2, IL-4 and IL-5). In order to create a single combined score, we create a linear discriminant analysis model where the response variable takes the following levels: “Untreared”, “ACE or Statin”, “ACE and Statin” and we use the first discriminant variate as a surrogate for a combined score. FIG. 18 presents the results from the subjects that are considered “Healthy” (“Controls”) as boxplots for each of the three “treatment” groups. The grey sections of each boxplot extend from the first to the third quantile of the value distribution for each class. The “notches:” around the medians are included for facilitating visual inspection of differences in the level of the median between the classes. The whiskers extend to 1.5 times the interquantile distance. The outliers have not been included in the graph. Clearly the combined score shows a downward trend with increased number of medications. The fact that the notches for the groups are barely overlapping indicates that the differences in the median are rather significant. A panel of biomarkers performs better than any single biomarker alone.

A similar analysis can be performed by creating a single score from multiple markers using Hottelling's T² method. In this case we can estimate the covariance matrix from the data for the untreated group and calculate the “distance” of each subject based on Hottelling's formula. The later approach can be used not only for creating a “combined distance” from many markers for monitoring medication dosage effect but also for hypothesis testing of the dosage effect. (see Hotelling, H. (1947). Multivariate Quality Control. In C. Eisenhart, M. W. Hastay, and W. A. Wallis, eds. Techniques of Statistical Analysis. New York: McGraw-Hill., herein incorporated by reference).

TABLE 7
ACE Inhibitor or Statin Prediction Model 1.
Logistic Regression

| Variables used: | mis-classification | AUC | sensitivity | specificity | accuracy |
|---|---|---|---|---|---|
| MCP-1, IGF-1, TNFa, MCP-2, IP10, IL- | 0.318 | 0.751 | 0.643 | 0.723 | 0.682 |
| 5, M-CSF, MCP-4, MCP-3, IL-3, Ang-2, | | | | | |
| IL-7, Eotaxin | | | | | |

TABLE 8
ACE Inhibitor or Statin Prediction Model 2.
Linear Discriminant Analysis

| Variables used: | mis-classification | AUC | sensitivity | specificity | accuracy |
|---|---|---|---|---|---|
| MCP-1, IGF-1, TNFa, MCP-2, IP10, IL-5, M- | 0.320 | 0.754 | 0.686 | 0.673 | 0.680 |
| CSF, MCP-4, MCP-3, IL-3, Ang-2, IL- | | | | | |
| 7, Eotaxin | | | | | |

### Example 8

### Coronary Calcium Score Prediction Models

Using the methods described in Example 5, we derived models using Logistic Regression or Linear Discriminant Analysis that classify samples according to a predicted coronary calcium score. The protein markers used in the exemplified models are set out in Tables 9 and 10, below, along with the models' performance characteristics.

TABLE 9
Coronary Calcium Score Prediction Model 1.
Logistic Regression

| Variables used: | mis-classification | AUCc | sensitivity | specificity | accuracy |
|---|---|---|---|---|---|
| MCP-1, IGF-1, TNFa, MCP-2, IP10, IL- | 0.470 | 0.536 | 0.567 | 0.500 | 0.530 |
| 5, M-CSF, MCP-4, MCP-3, IL-3, Ang-2, IL- | | | | | |
| 7, Eotaxin | | | | | |

TABLE 10
Coronary Calcium Score Prediction Model 2.
Linear Discriminant Analysis

| Variables used: | mis-classification | AUC | sensitivity | specificity | accuracy |
|---|---|---|---|---|---|
| MCP-1, IGF-1, TNFa, MCP-2, IP10, IL- | 0.461 | 0.560 | 0.578 | 0.505 | 0.539 |
| 5, M-CSF, MCP-4, MCP-3, IL-3, Ang-2, IL- | | | | | |
| 7, Eotaxin | | | | | |

### Example 9

### Stable vs. Unstable Atherosclerotic Disease Prediction Models

Using the methods described in Example 5, we derived models using Logistic Regression or Linear Discriminant Analysis that classify samples into stable (i.e., angina) or unstable (i.e., myocardial infarction) categories. The protein markers used in the exemplified models are set out in Tables 11 and 12, below, along with the models' performance characteristics.

TABLE 11
Stable vs. Unstable Disease Prediction Model 1.
Logistic Regression

| Variables used: | mis-classification | AUC | sensitivity | specificity | accuracy |
|---|---|---|---|---|---|
| MCP-1, IGF-1, TNFa, MCP-2, IP10, IL-5, M- | 0.438 | 0.566 | 0.563 | 0.562 | 0.562 |
| CSF, MCP-4, MCP-3, IL-3, Ang-2, IL- | | | | | |
| 7, Eotaxin | | | | | |

TABLE 12
Stable vs. Unstable Disease Prediction Model 2.
Linear Discriminant Analysis

| | mean | | | speci- | |
|---|---|---|---|---|---|
| Variables used: | cv error | AUC | sensitivity | ficity | accuracy |
| MCP-1, IGF-1, | 0.444 | 0.577 | 0.583 | 0.529 | 0.556 |
| TNFa, MCP-2, IP10, | | | | | |
| IL-5, M-CSF, MCP-4, | | | | | |
| MCP-3, IL-3, Ang-2, | | | | | |
| IL-7, Eotaxin | | | | | |

### Example 10

### Disease vs. Healthy Control Prediction Models

Using the methods described in Example 5, we derived models using Logistic Regression or Linear Discriminant Analysis that classify samples into disease (i.e., angina or myocardial infarction) or healthy control categories. The protein markers used in the exemplified models are set out in Tables 13 and 14, below, along with the models' performance characteristics. Tables 13 and 14 also indicate how the performance of the models change as combinations of markers are substituted.

TABLE 13
Disease vs. Control Prediction Model 1.
Linear Discriminant Analysis

| Variables used: | mis-classification | AUC | sensitivity | specificity | accuracy |
|---|---|---|---|---|---|
| MCP-1, IGF-1, TNFa, MCP-2, IP10, IL- | 0.158 | 0.915 | 0.847 | 0.840 | 0.842 |
| 5, M-CSF, MCP-4, MCP-3, IL-3, Ang-2, IL- | | | | | |
| 7, Eotaxin | | | | | |
| MCP-1, IGF-1, TNFa | 0.245 | 0.827 | 0.804 | 0.733 | 0.755 |
| MCP-1, IGF-1, M-CSF | 0.235 | 0.825 | 0.786 | 0.756 | 0.765 |
| Ang-2, IGF-1, M-CSF | 0.258 | 0.798 | 0.718 | 0.753 | 0.742 |
| MCP-4, IGF-1, M-CSF | 0.258 | 0.789 | 0.721 | 0.750 | 0.742 |
| MCP-1, IGF-1, TNFa, IL-5 | 0.225 | 0.850 | 0.817 | 0.757 | 0.775 |
| MCP-1, IGF-1, M-CSF, MCP-2 | 0.227 | 0.842 | 0.801 | 0.760 | 0.773 |
| Ang-2, IGF-1, M-CSF, IL-5 | 0.239 | 0.816 | 0.754 | 0.764 | 0.761 |
| MCP-1, IGF-1, TNFa, MCP-2 | 0.240 | 0.842 | 0.792 | 0.746 | 0.760 |
| MCP-1, IGF-1, TNFa, IL-5, M-CSF | 0.213 | 0.867 | 0.837 | 0.765 | 0.787 |
| MCP-1, IGF-1, IP10, MCP-2, M-CSF | 0.184 | 0.874 | 0.807 | 0.821 | 0.816 |
| Ang-2, IGF-1, TNFa, IL-5, M-CSF | 0.216 | 0.855 | 0.807 | 0.774 | 0.784 |
| MCP-1, IGF-1, TNFa, MCP-2, IP10 | 0.203 | 0.878 | 0.784 | 0.802 | 0.797 |
| MCP-4, IGF-1, M-CSF, TNFa, IL-5 | 0.221 | 0.855 | 0.812 | 0.765 | 0.779 |
| MCP-4, IGF-1, M-CSF, MCP-2, IL-5 | 0.246 | 0.807 | 0.736 | 0.761 | 0.754 |

TABLE 14
Disease vs. Control Prediction Model 2.
Logistic Regression

| Variables used: | mis-classification | AUC | sensitivity | specificity | accuracy |
|---|---|---|---|---|---|
| MCP-1, IGF-1, TNFa, MCP-2, IP10, IL- | 0.153 | 0.916 | 0.859 | 0.841 | 0.847 |
| 5, M-CSF, MCP-4, MCP-3, IL-3, Ang-2, IL- | | | | | |
| 7, Eotaxin | | | | | |
| MCP-1, IGF-1, TNFa | 0.237 | 0.835 | 0.804 | 0.745 | 0.763 |
| MCP-1, IGF-1, M-CSF | 0.239 | 0.831 | 0.789 | 0.749 | 0.761 |
| Ang-2, IGF-1, M-CSF | 0.257 | 0.799 | 0.734 | 0.747 | 0.743 |
| MCP-4, IGF-1, M-CSF | 0.258 | 0.792 | 0.733 | 0.745 | 0.742 |
| MCP-1, IGF-1, TNFa, IL-5 | 0.221 | 0.856 | 0.826 | 0.759 | 0.779 |
| MCP-1, IGF-1, M-CSF, MCP-2 | 0.236 | 0.845 | 0.794 | 0.750 | 0.764 |
| Ang-2, IGF-1, M-CSF, IL-5 | 0.243 | 0.813 | 0.766 | 0.754 | 0.757 |
| MCP-1, IGF-1, TNFa, MCP-2 | 0.235 | 0.849 | 0.784 | 0.757 | 0.765 |
| MCP-1, IGF-1, TNFa, IL-5, M-CSF | 0.212 | 0.868 | 0.832 | 0.769 | 0.788 |
| MCP-1, IGF-1, IP10, MCP-2, M-CSF | 0.187 | 0.876 | 0.804 | 0.816 | 0.813 |
| Ang-2, IGF-1, TNFa, IL-5, M-CSF | 0.220 | 0.855 | 0.801 | 0.771 | 0.780 |
| MCP-1, IGF-1, TNFa, MCP-2, IP10 | 0.202 | 0.881 | 0.794 | 0.799 | 0.798 |
| MCP-4, IGF-1, M-CSF, TNFa, IL-5 | 0.223 | 0.857 | 0.807 | 0.764 | 0.777 |
| MCP-4, IGF-1, M-CSF, MCP-2, IL-5 | 0.258 | 0.810 | 0.734 | 0.746 | 0.742 |

### Example 11

### Classification using an LDA Model

We classified a patient into a “Control” or “Disease” category based on the values of the following markers MCP-1, IGF-1 and TNFa. The costs of misclassification are taken to be equal for the two classes. Based on an LDA approach, a new subject with values x of the aforementioned markers is categorized into the “Disease” category if the left side of equation (1) is greater than the right side of the equation where:

a) index 2 corresponds to the “Disease” state

b) index 1 corresponds to the “Control” state

c) N is the total size of the training set

d) N1,N2 are the number of “Control” and “Disease” subjects in the training set

e) Σ is the covariance matrix as estimated from the training set

f) μ_{1,2} are the mean vectors of the “Control” and “Disease” sample respectively

$$x \text{?} \text{?} \sum \text{?} > \frac{1}{2} \text{?} \sum \text{?} - \frac{1}{2} \text{?} \sum \text{?} + log ( {N}_{1} / N ) log ( {N}_{2} / N ) \\ \text{?} \text{indicates text missing or illegible when filed} \qquad ( 1 )$$

In order to build an LDA model for the prediction we used a training set containing the three marker values for 398 subjects that were identified as “Control” and 398 subjects that were identified as “Disease.” The marker values are first log10 transformed and the resulting values are used to estimate the required terms of Eq. 1. The covariance matrix and mean marker vectors for the training set are equal to:

Covariance matrix:

| | MCP-1 | IGF-1 | TNFa |
|---|---|---|---|
| MCP-1 | 0.124155 | 0.069587 | 0.06659 |
| IGF-1 | 0.069587 | 1.321971 | 0.664374 |
| TNFa | 0.06659 | 0.664374 | 0.565535 |

Mean marker vectors for “Control” and “Disease” states:

| | | | |
|---|---|---|---|
| Control | 1.891552 | 2.830981 | 0.781913 |
| Disease | 1.223976 | 2.324683 | 0.990313 |

The inverse of the covariance matrix that is needed in equation 1 is:

| | V1 | V2 | V3 |
|---|---|---|---|
| 1 | 8.607599 | 0.13735 | −1.17487 |
| 2 | 0.13735 | 1.848967 | −2.18828 |
| 3 | −1.17487 | −2.18828 | 4.477304 |

We classified a subject with the following values (transformed using a log10 transformation):

Subject 1:

| MCP-1 | IGF-1 | TNFa |
|---|---|---|
| 0.716998 | 1.316101 | 0.287882 |

Based on these values and Eq. 1, the left side of the equation is equal to: 0.5291794 while the right side of the equation is equal to 3.232524. Based on the fact that the left side is less than the right side, the subject was classified into the “Control” category.

We classified a second subject with the following log10 transformed marker values:

Subject 2:

| MCP-1 | IGF-1 | TNFa |
|---|---|---|
| 1.991509 | 1.1113031 | 0.536339 |

Based on these values and using equation 1, the left side is equal to 4.461167 and the right hand side remains 3.232524. Based on this comparison the subject was classified into the “Disease” category.

Reference for this and the following example is made to “The elements of Statistical Learning. Data Mining, Inference and Prediction”, Hastie, T., Tibshirani, R., Friedman, J., Springer Series in Statistics, 2001), herein incorporated by reference.

### Example 12

### Classification using a Logistic Regression Model

We classified a patient into a “Control” or “Disease” category based on the values of the following markers MCP-1, IGF-1 and M-CSF. The costs of misclassification are taken to be equal for the two classes. Based on a Logistic Regression approach, a new subject with values x of the aforementioned markers will be categorized as Disease if the log ratio of the posterior probabilities of class k (=Disease) to class K(=Control) is greater than zero, otherwise it is categorized as Control (Equation 2).

$$log \frac{Pr ( G = k \vert X = x )}{Pr ( G = K \vert X = x )} = {β}_{k 0} + {β}_{k}^{T} x . \qquad ( 2 )$$

In order to fit a Logistic Regression model we used a training set composed of 398 subjects identified as “Control” and 398 subjects identified as “Disease.” The values of the three markers for each subject were first log10 transformed. The Logistic Regression fit provides the following coefficients:

| b0 | b1 | b2 | b3 |
|---|---|---|---|
| −4.95059 | 3.334 | −1.27675 | 1.279328 |

A new subject with the following values for the three markers was classified:

| | MCP-1 | IGF-1 | M-CSF |
|---|---|---|---|
| Subject 1 | 1.679931 | 3.493781 | 1.169145 |

The following calculation b0+b1*‘MCP-1’+b2*‘IGF-1’+b3*‘M-CSF’ equals −2.031. Based on the previous discussion this subject has a linear predictor value less than zero and was classified into the “Control” category.

Another subject was classified, based on the following values:

| | MCP-1 | IGF-1 | M-CSF |
|---|---|---|---|
| Subject 2 | 2.108252 | 1.7149 | 0.539566 |

Using the same coefficients and formula the linear predictor equals 0.5799186 and Subject 2 was classified into the “Disease” category.

Each publication cited in this specification is hereby incorporated by reference in its entirety for all purposes. In addition to those publications listed throughout the body of this specification, the following also is hereby incorporated by reference in its entirety for all purposes: Tabibiazar R, Wagner R A, Deng A, Tsao P S, Quertermous T. Proteomic profiles of serum inflammatory markers accurately predict atherosclerosis in mice. Physiol Genomics. 2006 Apr. 13; 25(2):194-202.

1. A method for classifying a sample obtained from a mammalian subject, comprising:
obtaining a dataset associated with said sample, wherein said dataset comprises quantitative data for at least three protein markers selected from the group consisting of MCP-1, MCP-2, MCP-3, MCP-4, eotaxin, IP-10, M-CSF, IL-3, TNFa, Ang-2, IL-5, IL-7, and IGF-1;
inputting said data into an analytical process that uses said data to classify said sample, wherein said classification is selected from the group consisting of an atherosclerotic cardiovascular disease classification, a healthy classification, a medication exposure classification, a no medication exposure classification; and
classifying said sample according to the output of said process.

2. The method of claim 1, wherein said analytical process comprises use of a predictive model.

3. The method of claim 1, wherein said analytical process comprises comparing said obtained dataset with a reference dataset.

4. The method of claim 3, wherein said reference dataset comprises data obtained from one or more healthy control subjects, or comprises data obtained from one or more subjects diagnosed with an atherosclerotic disease.

5. The method of claim 3, further comprising obtaining a statistical measure of a similarity of said obtained dataset to said reference dataset.

6. The method of claim 5, wherein said statistical measure is derived from a comparison of at least three parameters of said obtained dataset to corresponding parameters from said reference dataset.

7. The method of claim 1, wherein said at least three protein markers comprise a marker set selected from the group consisting of MCP-1, IGF-1, TNFa; MCP-1, IGF-1, M-CSF; ANG-2, IGF-1, M-CSF; and MCP-4, IGF-1, M-CSF.

8. The method of claim 1, wherein said dataset comprises quantitative data for at least four protein markers selected from the group consisting of MCP-1, MCP-2, MCP-3, MCP-4, eotaxin, IP-10, M-CSF, IL-3, TNFa, Ang-2, IL-5, IL-7, and IGF-1.

9. The method of claim 8, wherein said at least four protein markers comprise a marker set selected from the group consisting of MCP-1, IGF-1, TNFa, IL-5; MCP-1, IGF-1, M-CSF, MCP-2; ANG-2, IGF-1, M-CSF, IL-5; MCP-1, IGF-1, TNFa, MCP-2; and MCP-4, IGF-1, M-CSF, IL-5.

10. The method of claim 1, wherein said dataset comprises quantitative data for at least five markers selected from the group consisting of MCP-1, MCP-2, MCP-3, MCP-4, eotaxin, IP-10, M-CSF, IL-3, TNFa, Ang-2, IL-5, IL-7, and IGF-1.

11. The method of claim 10, wherein said at least five protein markers are selected from the group consisting of MCP-1, IGF-1, TNFa, IL-5, M-CSF; MCP-1, IGF-1, M-CSF, MCP-2, IP-10; ANG-2, IGF-1, M-CSF, IL-5, TNFa; MCP-1, IGF-1, TNFa, MCP-2, IP-10; MCP-4, IGF-1, M-CSF, IL-5, TNFa; and MCP-4, IGF-1, M-CSF, IL-5, MCP-2.

12. A method for classifying a sample obtained from a mammalian subject, comprising:
obtaining a dataset associated with said sample, wherein said dataset comprises quantitative data for at least three protein markers selected from the group consisting of MCP1; MCP2; MCP3; MCP4; Eotaxin; IP10; MCSF; IL3; TNFα; Ang2; IL5; IL7; IGF1; IL10; INFγ; VEGF; MIP1a; RANTES; IL6; IL8; ICAM; TIMP1; CCL19; TCA4/6kine/CCL21; CSF3; TRANCE; IL2; IL4; IL13; Il1b; MCP5; CCL9; CXCL1/GRO1; GROalpha; IL12; and Leptin;
inputting said data into a predictive model that uses said data to classify said sample, wherein said classification is selected from the group consisting of an atherosclerotic cardiovascular disease classification, a healthy classification, a medication exposure classification, a no medication exposure classification, wherein said predictive model has at least one quality metric of at least 0.7 for classification; and
classifying said sample according to the output of said predictive model.

13. The method of claim 12, wherein said predictive model has a quality metric of at least 0.8 for classification.

14. The method of claim 13, wherein said predictive model has a quality metric of at least 0.9 for classification.

15. The method of claim 12, wherein said quality metric is selected from AUC and accuracy.

16. The method of claim 12, wherein the limits of said predictive model are adjusted to provide at least one of sensitivity or specificity of at least 0.7.

17. The method of claim 14, wherein the limits of said predictive model are adjusted to provide at least one of sensitivity or specificity of at least 0.7.

18. The method of claim 1, wherein said atherosclerotic disease classification is selected from the group consisting of coronary artery disease, myocardial infarction, and angina.

19. The method of claim 1, further comprising using said classification for atherosclerosis diagnosis, atherosclerosis staging, atherosclerosis prognosis, vascular inflammation levels, assessing extent of atherosclerosis progression, monitoring a therapeutic response, predicting a coronary calcium score, or distinguishing stable from unstable manifestations of atherosclerotic disease.

20. The method of claim 1, wherein said dataset further comprises data for one or more clinical indicia.

21. The method of claim 20, wherein said one or more clinical indicia are selected from the group consisting of age, gender, LDL concentration, HDL concentration, triglyceride concentration, blood pressure, body mass index, CRP concentration, coronary calcium score, waist circumference, tobacco smoking status, previous history of cardiovascular disease, family history of cardiovascular disease, heart rate, fasting insulin concentration, fasting glucose concentration, diabetes status, and use of high blood pressure medication.

22. The method of claim 1, wherein said sample comprises blood or a blood derivative.

23. The method of claim 1, wherein said analytic process comprises using a Linear Discriminant Analysis model, a support vector machine classification algorithm, a recursive feature elimination model, a prediction analysis of microarray model, a Logistic Regression model, a CART algorithm, a FlexTree algorithm, a LART algorithm, a random forest algorithm, a MART algorithm, or Machine Learning algorithms.

24. The method of claim 23, wherein said process comprises using a Linear Discriminant Analysis model or a Logistic Regression model, and said model comprises terms selected to provide a quality metric greater than 0.75.

25. The method of claim 1, further comprising obtaining a plurality of classifications for a plurality of samples obtained at a plurality of different times from said subject.

26. A method for classifying a sample obtained from a mammalian subject, comprising:
obtaining a dataset associated with said sample, wherein said dataset comprises quantitative data for at least three protein markers that each shows a correlation between a circulating protein concentration and an atherosclerotic vascular tissue RNA concentration;
inputting said data into an analytical process that uses said data to classify said sample, wherein said classification is selected from the group consisting of an atherosclerotic cardiovascular disease classification, a healthy classification, a medication exposure classification, a no medication exposure classification; and
classifying said sample according to the output of said process.

27. The method of claim 26, wherein said correlation is characterized by a Pearson correlation coefficient of at least 0.6.

28. The method of claim 27, wherein said at least three protein markers comprise one or more protein markers selected from the set consisting of MCP-1, CCL21, CCL19, CCL112, TNFSF11, and CCL11.

29. The method of claim 26, wherein said mammalian subject is a human subject.

30. A method for classifying a sample obtained from a mammalian subject, comprising:
obtaining a dataset associated with said sample, wherein said dataset comprises quantitative data for at least three protein markers that each shows a correlation between a circulating protein concentration and an atherosclerotic vascular tissue RNA concentration,
inputting said data into a predictive model that uses said data to classify said sample, wherein said classification is selected from the group consisting of an atherosclerotic cardiovascular disease classification, a healthy classification, a medication exposure classification, a no medication exposure classification, wherein said predictive model has at least one quality metric of at least 0.7 for classification; and
classifying said sample according to the output of said predictive model.

31. The method of claim 30, wherein said correlation is characterized by a Pearson correlation coefficient of at least 0.6.

32. The method of claim 31, wherein said at least three protein markers comprise one or more protein markers selected from the set consisting of MCP-1, CCL21, CCL19, CCLl12, TNFSF11, and CCL11.

33. The method of claim 30, wherein said mammalian subject is a human subject.
